(19) United States
(12) Patent Application Publication
(10) Pub. No.: US 2024/0307412 A1
(43) Pub. Date: Sep. 19, 2024

(54) METHODS FOR TREATING INFLAMMATORY PAIN AND BONE DECALCIFICATION

(71) Applicant: Dyve Biosciences, Inc., Camarillo, CA (US)

(72) Inventor: Ryan Beal, Camarillo, CA (US)

(21) Appl. No.: 18/670,600

(22) Filed: May 21, 2024

Related U.S. Application Data
(63) Continuation of application No. PCT/US22/80368, filed on Nov. 22, 2022.
(60) Provisional application No. 63/282,106, filed on Nov. 22, 2021.
(60) Provisional application No. 63/282,114, filed on Nov. 22, 2021.

(51) Int. Cl. A61K 31/573; A61K 9/00; A61K 45/06; A61K 47/10; A61P 19/06

(52) U.S. Cl. CPC A61K 31/573; A61K 9/0014; A61K 9/0053; A61K 45/06; A61K 47/10; A61P 19/06

(57) ABSTRACT

The present disclosure relates to transdermal formulations, compositions, and methods for treating or preventing inflammation or a symptom thereof, e.g., inflammatory pain.

### CROSS-REFERENCE

This application is a continuation of PCT Application No. PCT/US2022/080368 filed on Nov. 22, 2022, which claims the benefit of U.S. 63/282,106 and to U.S. 63/282,114, each filed Nov. 22, 2021; the contents of each of which is incorporated herein by reference in its entirety.

### BACKGROUND

Inflammation is a natural response to microbial, autoimmune, metabolic, or physical traumas which are experienced by a subject, and are important aspects of the body's healing and immune system responses. Aspects of inflammation include overproduction of white blood cells, activation of macrophages, production of cytokines, activation of neutrophils, eosinophils, lymphocytes, plasma cells, myeloid granulocytes, and histocytes, increased blood flow, swelling, stiffness fatigue, fever, and pain, among other symptoms. In the short-term following such a trauma, acute inflammation experienced by a subject offers a physiological benefit by assisting the body in fighting infection and speeding the healing process. In a normal healthy response, inflammation becomes activated, clears a pathogen (where applicable), begins a repair process, and then ceases.

However, when an inflammatory response fails to recede following trauma, inflammation becomes chronic and often results in negative physiological consequences. Chronic inflammation will eventually begin to damage the body's healthy cells tissues, and organs, possibly leading to DNA damage, tissue death and internal scarring. Although, there are numerous anti-inflammatories that treat or prevent inflammation, and especially, inflammation-related pain, there remains a need for improved therapeutics and methods.

### SUMMARY OF THE INVENTION

The present disclosure addresses this need. Accordingly, the present disclosure relates to transdermal formulations, compositions, and methods for treating or preventing inflammation or a symptom thereof, e.g., inflammatory pain.

An aspect of the present disclosure is a method for method for treating or reducing inflammation in a subject in need thereof. The method comprising administering to the subject a transdermal formulation comprising a therapeutically-effective amount of a buffering agent. In this method, the transdermal formulation reduces blood or serum levels of C-Reactive Protein relative to a subject that is not administered the combination therapy.

In embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In some embodiments, administering the transdermal formulation further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

Another aspect of the present disclosure is a method for treating or reducing inflammation in a subject in need thereof. The method comprises administering to the subject a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy.

In various embodiments, the separate composition is administered orally or topically before, contemporaneously with, and/or after administering the transdermal formulation.

In embodiments, the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In some embodiments, administering the combination therapy further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

In various embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

A further aspect of the present disclosure is a method for treating or reducing inflammation in a subject in need thereof, the method comprising administering to the subject a transdermal formulation comprises a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

In embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In some embodiments, administering the transdermal formulation further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

In various embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

An additional aspect of the present disclosure is a method for preventing inflammation and/or reducing the severity of upcoming inflammation, the method comprising administering to a subject who is not experiencing a symptom of inflammation a transdermal formulation comprising a therapeutically-effective amount of a buffering agent. In this method, the transdermal formulation reduces blood or serum levels of C-Reactive Protein relative to a subject that is not administered the combination therapy.

In embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In an aspect, the present disclosure provides a method for preventing inflammation and/or reducing the severity of upcoming inflammation, the method comprising administering to a subject who is not experiencing a symptom of inflammation, a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy

In some embodiments, the separate composition is administered orally or topically before, contemporaneously with, and/or after administering the transdermal formulation.

In various embodiments, the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In another aspect, the present disclosure provides a method for preventing inflammation and/or reducing the severity of upcoming inflammation, the method comprising administering to a subject who is not experiencing a symptom of inflammation, a transdermal formulation comprising a therapeutically-effective amount of a buffering agent and a therapeutically-effective amount of a nonsteroidal medicament and/or a corticosteroid. In this method, the transdermal formulation is administered before symptoms inflammation are experienced by the subject and prevents inflammation and/or reduces the severity of upcoming inflammation is determined in part by a reduction of blood or serum levels of C-Reactive Protein relative to a subject that is not administered the combination therapy.

In some embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In various embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In a further aspect, the present disclosure provides a method for reducing the likelihood a recurrent inflammation, the method comprising administering to a subject that previously has been treated for inflammation, a transdermal formulation comprising a therapeutically-effective amount of a buffering agent.

In this method, the transdermal formulation reduces blood or serum levels of C-Reactive Protein relative to a subject that is not administered the combination therapy.

In embodiments, the dosage of the therapeutically-effective amount of the buffering agent is less than the dosage used to previously treat inflammation.

In some embodiments, the dosage of the therapeutically-effective amount of the buffering agent is the same as the dosage used to previously treat inflammation.

In various embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In an additional aspect, the present disclosure provides a method for reducing the likelihood a recurrent inflammation, the method comprising administering to a subject that previously has been treated for inflammation, a transdermal formulation comprising a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

In embodiments, the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In some embodiments, the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In various embodiments, another therapeutically-effective amount of the nonsteroidal medicament and/or the corticosteroid is administered orally or topically before, contemporaneously with, and/or after administering the transdermal formulation.

In embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In some embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In yet another aspect, the present disclosure provides a method for reducing the likelihood a recurrent inflammation, the method comprising administering to a subject that previously has been treated for inflammation, administering to the subject a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the transdermal formulation reduces blood or serum levels of C-Reactive Protein relative to a subject that is not administered the combination therapy.

In various embodiments, the separate composition is administered orally or topically before, contemporaneously with, and/or after administering the transdermal formulation.

In embodiments, the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In some embodiments, the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In various embodiments, the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In yet a further aspect, the present disclosure provides a method for treating chronic inflammation, the method comprising administering to a subject that previously has been treated for inflammation, a transdermal formulation comprising a therapeutically-effective amount of a buffering agent. In this method, the transdermal formulation reduces blood or serum levels of C-Reactive Protein relative to a subject that is not administered the combination therapy.

In some embodiments, the therapeutically-effective amount of the buffering agent is less than the dosage used to previously treat inflammation.

In various embodiments, the dosage of the therapeutically-effective amount of the buffering agent is the same as the dosage used to previously treat inflammation.

In embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In yet another aspect, the present disclosure provides a method for treating chronic inflammation, the method comprising administering to a subject that previously has been treated for inflammation, a transdermal formulation comprising a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

In some embodiments, the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In various embodiments, the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In embodiments, another therapeutically-effective amount of the nonsteroidal medicament and/or the corticosteroid is administered orally or topically before, contemporaneously with, and/or after administering the transdermal formulation.

In some embodiments, the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In various embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In yet an additional aspect, the present disclosure provides a method for treating chronic inflammation, the method comprising administering to a subject that previously has been treated for inflammation, a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy.

In embodiments, the separate composition is administered orally or topically before, contemporaneously with, and/or after administering the transdermal formulation.

In some embodiments, the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In various embodiments, the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

In embodiments, the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

In some embodiments, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

An aspect of the present disclosure is a method for treating a bone density disorder in a subject in need thereof, the method comprising administering to the subject a transdermal formulation comprising a therapeutically-effective amount of a buffering agent. In this method, the transdermal formulation lowers elevated calcium levels in blood or plasma, stabilizes calcium levels in blood or plasma to levels prior to an inflammatory event, reduces symptoms related to osteoporosis, reduces symptoms related to osteomalacia, improves bone density, and/or inhibits and/or reverses bone decalcification.

In various embodiments, the transdermal formulation comprises a penetrant or penetration enhancer comprising one or more of phosphatidyl choline (e.g., Phospholipon® 90 G), isopropyl palmitate (IPP), steric acid, benzyl alcohol, safflower oil, almond oil, oleic acid, polyglyceryl-4 laurate, poloxamer 407, poloxamer 188, poloxamer 124, menthol, propylene glycol, cetyl alcohol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin.

In embodiments, the transdermal formulation comprises propylene glycol.

In some embodiments, the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

In various embodiments, the transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

In embodiments, when the nonsteroidal medicament and/or the corticosteroid is included in the transdermal formulation, the amount of deionized water is reduced to provide for the addition of the therapeutically-effective amount of the nonsteroidal medicament and/or the corticosteroid.

In some embodiments, the concentration of the buffering agent is from about 10% to about 50% w/w of the transdermal formulation The method of any one of Embodiment B1 to Embodiment B57, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

In various embodiments, the sodium bicarbonate or sodium carbonate is at a concentration from about 30% to about 35% w/w of the transdermal formulation, e.g., the sodium bicarbonate or sodium carbonate is at a concentration of about 33% w/w of the transdermal formulation.

In embodiments, the transdermal formulation comprises menthol, e.g., at a concentration from about 0.1% to about 5.0% w/w of the transdermal formulation.

In some embodiments, the transdermal formulation comprises about 33% w/w sodium bicarbonate or sodium carbonate and about 0.5% w/w menthol.

In various embodiments, the transdermal formulation has a pH from about 9 to about 11 or from about 7 to about 10.5.

In embodiments, the method comprises administering the transdermal formulation about three times a day.

In some embodiments, the transdermal formulation is formulated as a cream, lotion, or ointment.

In various embodiments, the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

In embodiments, the transdermal formulation is as described in any of Table 1 to Table 19 or as described elsewhere herein.

In some embodiments, the subject in need thereof is further administered a separate composition comprising a therapeutically-effective amount of colchicine, wherein the colchicine is administered before, contemporaneously with, and/or after administering the transdermal formulation.

In various embodiments, the therapeutically-effective amount of colchicine is administered orally.

In embodiments, the therapeutically-effective amount of colchicine is administered topically.

In some embodiments, the transdermal formulation comprising a therapeutically-effective amount of a buffering agent further comprises a therapeutically-effective amount of colchicine.

In various embodiments, the dosage of the therapeutically-effective amount of colchicine is less than the dosage used to previously treat a gout flare or the therapeutically-effective amount of colchicine is the same as the dosage used to previously treat a gout flare.

In embodiments, the therapeutically-effective amount of colchicine comprises from about 0.2 mg to about 4 mg, e.g., about 0.3 mg to about 3.6 mg and/or be present in an amount from 0.02% to about 0.4% w/w of the formulation, e.g., about 0.03% to about 0.36% w/w.

In some embodiments, the transdermal formulation is as described in any of Table 1 to Table 19 or as described elsewhere herein.

An aspect of the present disclosure is a transdermal formulation for use in method of treating or reducing inflammation in a subject in need thereof, the transdermal formulation comprising a therapeutically-effective amount of a buffering agent and comprising a therapeutically-effective amount of a nonsteroidal medicament and/or a corticosteroid. The transdermal formulation reduces blood or serum levels of C-Reactive Protein relative to a subject that is not administered the combination therapy.

Another aspect of the present disclosure is a transdermal formulation for use in method of preventing inflammation and/or reducing the severity of upcoming inflammation, the transdermal formulation comprising a therapeutically-effective amount of a buffering agent and comprising a therapeutically-effective amount of a nonsteroidal medicament and/or a corticosteroid. In this aspect, the transdermal formulation is administered before symptoms of inflammation are experienced by the subject and preventing inflammation and/or reducing the severity of upcoming inflammation is determined in part by a reduction of blood or serum levels of C-Reactive Protein relative to a subject that is not administered the combination therapy.

A further aspect of the present disclosure is a transdermal formulation for use in method of reducing the likelihood a recurrent inflammation, the method comprising administering to a subject that previously has been treated for inflammation, the transdermal formulation comprising a therapeutically-effective amount of a buffering agent and comprising a therapeutically-effective amount of a nonsteroidal medicament and/or a corticosteroid. In this aspect, the transdermal formulation is administered before symptoms of inflammation are experienced by the subject and reducing the likelihood of a recurrent inflammation is determined in part by a reduction of blood or serum levels of C-Reactive Protein relative to a subject that is not administered the combination therapy.

An additional aspect of the present disclosure is a transdermal formulation for use in method of treating chronic inflammation, the method comprising administering to a subject that previously has been treated for inflammation, the transdermal formulation comprising a therapeutically-effective amount of a buffering agent and comprising a therapeutically-effective amount of a nonsteroidal medicament and/or a corticosteroid. In this aspect, treating chronic inflammation is determined in part by a reduction of blood or serum levels of C-Reactive Protein relative to a subject that is not administered the combination therapy.

In an aspect, the present disclosure provides a transdermal formulation for use in method of treating a bone density disorder in a subject in need thereof. The method comprising administering to the subject, the transdermal formulation comprising a therapeutically-effective amount of a buffering agent.

In another aspect, the present disclosure provides a transdermal formulation for use in method of treating or reducing inflammation in a subject in need thereof, the transdermal formulation comprising a therapeutically-effective amount of a buffering agent and comprising a therapeutically-effective amount of colchicine. In this aspect, the transdermal formulation reduces blood or serum levels of C-Reactive Protein relative to a subject that is not administered the combination therapy.

In various embodiments, the transdermal formulation further reduces or eliminates the need for a rescue medicine; improves the subject's physical function as measured by a Patient-Reported Outcomes Measurement Information System (PROMIS) score, e.g., PROMIS PF 20; provides an improvement in the subject's Sum of Pain Intensity Difference (SPID) score; lowers the subject's pain-numeric rating; decreases the time to resolution of pain relative to a historical control patient; lowers the subject-reported or physician-assessed moderate-to-severe joint tenderness; lowers the subject-reported or physician-assessed moderate-to-severe joint swelling; reduces uric acid crystal levels in blood or plasma; raises urine pH, and/or increases patient satisfaction.

In a further aspect, the present disclosure provides a transdermal formulation for use in method of preventing inflammation and/or reducing the severity of upcoming inflammation, the transdermal formulation comprising a therapeutically-effective amount of a buffering agent and comprising a therapeutically-effective amount of colchicine. In this aspect, the transdermal formulation is administered before symptoms of inflammation are experienced by the subject and preventing inflammation and/or reducing the severity of upcoming inflammation is determined in part by a reduction of blood or serum levels of C-Reactive Protein relative to a subject that is not administered the combination therapy.

In an additional aspect, the present disclosure provides a transdermal formulation for use in method of reducing the likelihood a recurrent inflammation, the method comprising administering to a subject that previously has been treated for inflammation, the transdermal formulation comprising a therapeutically-effective amount of a buffering agent and comprising a therapeutically-effective amount of colchicine. In this aspect, the transdermal formulation is administered before symptoms of inflammation are experienced by the subject and reducing the likelihood of a recurrent inflammation is determined in part by a reduction of blood or serum levels of C-Reactive Protein relative to a subject that is not administered the combination therapy.

An aspect of the present disclosure is a transdermal formulation for use in method of treating chronic inflammation, the method comprising administering to a subject that previously has been treated for inflammation, the transdermal formulation comprising a therapeutically-effective amount of a buffering agent and comprising a therapeutically-effective amount of colchicine. In this aspect, treating chronic inflammation is determined in part by a reduction of blood or serum levels of C-Reactive Protein relative to a subject that is not administered the combination therapy.

Another aspect of the present disclosure is a transdermal formulation for use in method of treating a bone density disorder in a subject in need thereof. The method comprising administering to the subject, the transdermal formulation comprising a therapeutically-effective amount of a buffering agent and comprising a therapeutically-effective amount of colchicine.

In embodiments, the transdermal formulation is as described in any of Table 1 to Table 19 or as described elsewhere herein.

It shall be understood that different aspects and/or embodiments of the present disclosure can be appreciated individually, collectively, or in combination with each other. Any description herein concerning a specific formulation, composition, and/or method apply to and may be used for any other specific formulation, composition, and/or method as disclosed herein. Additionally, any formulation or composition disclosed herein is applicable to any herein-disclosed method. In other words, any aspect or embodiment described herein can be combined with any other aspect or embodiment as disclosed herein.

### BRIEF DESCRIPTION OF THE DRAWINGS

FIG. 1 is a graph showing a timeline of compliance of subjects in the study described in Example 1.

FIG. 2 includes graphs showing results for the Primary Endpoint—Improved SPID_{0-7 days} for the study subjects who received transdermal formulations of the present disclosure.

FIG. 3 includes graphs showing an improvement in overall responder rates for the study subjects who received transdermal formulations of the present disclosure.

FIG. 4 includes a graph showing improved time to resolution of pain for the Per Protocol subject population that received transdermal formulations of the present disclosure.

FIG. 5 includes graphs showing an improved 24-hour response rate for the study subjects who received transdermal formulations of the present disclosure.

FIG. 6 includes graphs showing a reduction in use of rescue medications for the study subjects who received transdermal formulations of the present disclosure.

FIG. 7 includes graphs showing an improvement in Patient-Rated Physical Function (PROMIS PF-20) by 24 hours for the study subjects who received transdermal formulations of the present disclosure.

FIG. 8 includes graphs showing an improvement in Patient-Rated Physical Function (PROMIS PF-20) for the study subjects who received transdermal formulations of the present disclosure.

FIG. 9 includes graphs showing reduction in moderate/severe tenderness at 24 hours for the study subjects who received transdermal formulations of the present disclosure.

FIG. 10 includes graphs showing reduction in moderate/severe swelling at 24 hours for the study subjects who received transdermal formulations of the present disclosure.

FIG. 11 includes graphs showing changes in C-Reactive Proteins for the study subjects who received transdermal formulations of the present disclosure.

FIG. 12 includes a graph showing changes in serum calcium (left image) and in C-Reactive Proteins (right image) for the study subjects who received transdermal formulations of the present disclosure.

### DETAILED DESCRIPTION

The present disclosure relates to transdermal formulations, compositions, and methods for treating or preventing inflammation or a symptom thereof, e.g., inflammatory pain.

Inflammation is a natural response to microbial, autoimmune, metabolic, or physical traumas which are experienced by a subject, and are important aspects of the body's healing and immune system responses.

Aspects of inflammation include overproduction of white blood cells, activation of macrophages, production of cytokines, activation of neutrophils, eosinophils, lymphocytes, plasma cells, myeloid granulocytes, and histocytes, increased blood flow, swelling, stiffness fatigue, fever, and pain, among other symptoms. In the short-term following such a trauma, acute inflammation experienced by a subject offers a physiological benefit by assisting the body in fighting infection and speeding the healing process. In a normal healthy response, inflammation becomes activated, clears a pathogen (where applicable), begins a repair process, and then ceases.

However, when an inflammatory response fails to recede following trauma, inflammation becomes chronic and often results in negative physiological consequences. Chronic inflammation will eventually begin to damage the body's healthy cells tissues, and organs, possibly leading to DNA damage, tissue death and internal scarring. In addition, a number of lifestyle factors are thought to influence levels of chronic inflammation in a subject including chronic stress; alcohol abuse; smoking; and obesity, among other factors.

A common indicator of chronic inflammation is a subject having clinically significant elevated levels of C-reactive protein. C-reactive protein is a protein produced in the liver and present in the bloodstream during an inflammatory response. Subject CRP levels rise or fall depending on the amount of ongoing inflammation experienced by the subject. A subject experiencing clinically elevated levels of CRP indicates the present of ongoing inflammation which may have a negative physiological effect. In some cases, reducing a subject's c-reactive protein levels may treat a symptom of an inflammatory condition. In some cases, the inflammatory condition may be Alzheimer's; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Chron's disease; cancer; chronic stress; alcohol abuse; smoking; or obesity; and combinations thereof.

Without being bound to a particular theory, slowing of inflammatory related disease progression by reducing inflammation and immune system dysregulation, can possibly be accomplished by reducing subject CRP levels, and by returning macrophages to their inactivated state; as high and increasing CRP levels among the subjects is indicative of inflammation and immune system dysregulation, while the low and steady CRP levels among subjects indicates there may not be clinically significant inflammation and immune system dysregulation. A reduction in leukocyte infiltration, inflammatory mediators, pro-apoptotic factors, and matrix degrading proteases as a result of macrophage over activation may contribute to reduction in symptoms, tissue damage, and cell death associated with chronic inflammation.

The transdermal formulations and methods thereof of the present disclosure reduces blood or serum levels of C-Reactive Protein to create an on demand, fast-acting, simple, safe non-biologic that provides anti-inflammatory effects. Indeed, the transdermal formulations of the present disclosure reduce the pain intensity and duration of inflammation. This can lead to significant improvements in physical function and reductions in rescue medication use. The efficacy profile and lack of adverse effects makes the transdermal formulations of the present disclosure a superior therapeutic choice; especially during debilitating inflammatory pain in patients with concomitant comorbidities. Moreover, the transdermal formulations of the present disclosure are a safe, nonbiologic that is compatible with other therapeutics taken by those experiencing chronic inflammation, who have high degree of concomitant comorbidities. The transdermal formulations of the present disclosure have fast onset of action and should decrease ambulatory visits, emergency department visits, and hospitalizations. Overall, the transdermal formulations and methods of the present disclosure provide a clear and unexpected improvement over the standard of care for pain associated with inflammation and, especially, resulting from chronic inflammation.

Indeed, as disclosed in the Examples, a most surprising result of the described study was a reduction in C-Reactive Protein levels in subjects who received the transdermal formulation of the present disclosure. This reduction in C-Reactive Protein levels has implications for treating and prevention the numerous types of inflammation that are associated with C-Reactive Protein, e.g., with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity.

Additionally, the formulations and methods of the present disclosure prevent and/or reduce bone decalcification, which least to a relative decrease in serum calcium levels. It is known in the art that increases in serum calcium levels are typically derived from bone decalcification. As disclosed herein, the transdermal formulations of the present disclosure provide systemic buffering, which contribute to treating an inflammation, e.g., inflammatory pain. Importantly, this systemic buffering avoids the body's need to decalcify the bones to obtain serum calcium in the context of inflammation. Without wishing to be bound by theory, the formulations and methods of the present disclosure may prevent bone decalcification due to other pathological conditions, such as osteomalacia and osteoporosis, and in the absence of an inflammation, e.g., inflammatory pain. Thus, the formulations and methods of the present disclosure lower elevated calcium levels in blood or plasma, stabilize calcium levels in blood or plasma to levels prior to inflammation, e.g., inflammatory pain, reduce symptoms related to osteoporosis, reduce symptoms related to osteomalacia, improve bone density, and/or inhibit and/or reverse bone decalcification.

Notably, some formulations, compositions, and/or methods of the present disclosure do not comprise colchicine. Mild side effects of colchicine include diarrhea, nausea, cramping, abdominal pain, and vomiting and serious side effects include unusual bleeding/bruising, severe diarrhea or vomiting, muscle weakness or pain, numbness/tingling in your fingers or toes, pale or gray color of the lips/tongue/palms of hands, signs of infection (such as fever, persistent sore throat), unusual weakness/tiredness, fast heartbeat, shortness of breath, and signs of kidney problems (such as change in the amount of urine). Importantly, colchicine may cause a certain serious (even fatal) muscle damage (rhabdomyolysis). This muscle damage releases substances that can lead to serious kidney problems. Although not common, there are some studies showing direct toxicity of the kidneys due to the use of colchicine. These report kidney damage as well as the potential for kidney failure from the direct toxic effect of colchicine on the kidney tubules. Indeed, colchicine use is counter indicated for subjects with kidney impairment, for example in subjects with diabetes, chronic kidney disease (CKD), Polycystic kidney disease (PKD), Lupus nephritis, kidney cancer, Alport syndrome, amyloidosis, Goodpasture syndrome, and Wegener's granulomatosis, and/or recipients of a renal transplant.

Since the formulations, compositions, and/or methods of the present disclosure do not comprise colchicine, they may be safely used in subjects with kidney impairment.

### Introduction

Topical administration describes the application of a substance to a surface of the skin. The term is often used to describe the application of a cream, foam, gel, lotion or ointment to the skin or mucous membranes. The high keratinization of skin cells and their dense packing creates, in most cases, a barrier impermeable to penetration. Because of this, many substances are not absorbed through the skin.

The term transdermal administration refers to applying a substance onto the skin so that it is absorbed into the body for local or systemic distribution. A transdermal solution or transdermal patch is typically placed on one's skin. The solution or patch includes a medicament that is released into the skin. As the layers of skin absorb the solution, the medicament is absorbed via the blood vessels into the bloodstream. From there, the substance can be circulated through the body.

There are obvious advantages to transdermal administration of medicaments. The consumer does not have to schedule and remember to consume doses of pills. Use of a convenience transdermal formulation helps improve compliance. Also, patients having inflammation-related pain can benefit from a medicament or medicaments that are absorbed slowly and regularly. With a transdermal formulation, a medicament can be released in small quantities over a long period of time.

Other advantages are related to dosing. Large doses of some medicaments can cause dose-dependent toxicity in many cases. With a transdermal formulation, the effective concentration of an agent can be applied at the desired site without painful delivery.

The transdermal formulations of the present disclosure (e.g., as a lotion, ointment, or cream) are placed on the skin to deliver a specific dose of a medicament (e.g., buffering agent) or medicaments (the buffering agent along with another medicament, such as colchicine) through the skin. The lotion or cream can be applied directly to the affected area. A medicament is delivered across the skin into a localized subdermal location. Alternatively, the medicament can enter the circulation for systemic distribution. In some cases, the transdermal formulation is administered using a transdermal patch or medicated adhesive patch. To release an agent, a patch can utilize a porous membrane covering a reservoir of the medicament.

Alternatively, the medicament can be embedded in layers of the adhesive that release the medicament as they dissolve or melt.

The present disclosure relates to transdermal formulations and methods of use that comprise a buffering agent, e.g., sodium bicarbonate and sodium carbonate. The transdermal formulations may further comprise colchicine. Alternately or additionally, the transdermal formulations may further comprise another medicament including a nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase). In some cases, the transdermal formulation comprises menthol and in other cases, the transdermal formulation lacks menthol.

Additionally, the formulations and methods of the present disclosure prevent bone decalcification due to pathological conditions, such as gout, osteomalacia, and osteoporosis, and in the absence of an inflammation, e.g., inflammatory pain. The formulations and methods of the present disclosure lower elevated calcium levels in blood or plasma, stabilize calcium levels in blood or plasma to levels prior to an inflammatory event, reduce symptoms related to osteoporosis, reduce symptoms related to osteomalacia, improve bone density, and/or inhibit and/or reverse bone decalcification.

In any aspect or embodiment, the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

### Illustrative Transdermal Formulations

An aspect of the present disclosure is a transdermal formulation for use in method of treating or reducing inflammation in a subject in need thereof: The transdermal formulation comprises a therapeutically-effective amount of a buffering agent and a therapeutically-effective amount of colchicine. This transdermal formulation reduces blood or serum levels of C-Reactive Protein relative to a subject that is not administered the combination therapy.

Another aspect of the present disclosure is a transdermal formulation for use in method of preventing inflammation and/or reducing the severity of upcoming inflammation: The transdermal formulation comprises a therapeutically-effective amount of a buffering agent and comprises a therapeutically-effective amount of colchicine: This transdermal formulation is administered before symptoms of inflammation are experienced by the subject. With this transdermal formulation, preventing inflammation and/or reducing the severity of upcoming inflammation is determined in part by a reduction of blood or serum levels of C-Reactive Protein relative to a subject that is not administered the combination therapy.

A further aspect of the present disclosure is a transdermal formulation for use in method of reducing the likelihood a recurrent inflammation, the method comprising administering to a subject that previously has been treated for inflammation: The transdermal formulation comprises a therapeutically-effective amount of a buffering agent and comprises a therapeutically-effective amount of colchicine: This transdermal formulation is administered before symptoms of inflammation are experienced by the subject. With this transdermal formulation, reducing the likelihood of a recurrent inflammation is determined in part by a reduction of blood or serum levels of C-Reactive Protein relative to a subject that is not administered the combination therapy.

An additional aspect of the present disclosure is a transdermal formulation for use in method of treating chronic inflammation, the method comprising administering to a subject that previously has been treated for inflammation: The transdermal formulation comprises a therapeutically-effective amount of a buffering agent and comprises a therapeutically-effective amount of colchicine. With this transdermal formulation, treating chronic inflammation is determined in part by a reduction of blood or serum levels of C-Reactive Protein relative to a subject that is not administered the combination therapy.

In an aspect, the present disclosure provides a transdermal formulation for use in method of treating a bone density disorder in a subject in need thereof. The method comprising administering to the subject, the transdermal formulation comprising a therapeutically-effective amount of a buffering agent and comprising a therapeutically-effective amount of colchicine.

In another aspect, the present disclosure provides a transdermal formulation for use in method of treating or reducing inflammation in a subject in need thereof, the transdermal formulation comprising a therapeutically-effective amount of a buffering agent and comprising a therapeutically-effective amount of a nonsteroidal medicament and/or a corticosteroid: The transdermal formulation reduces blood or serum levels of C-Reactive Protein relative to a subject that is not administered the combination therapy.

In a further aspect, the present disclosure provides a transdermal formulation for use in method of preventing inflammation and/or reducing the severity of upcoming inflammation, the transdermal formulation comprising a therapeutically-effective amount of a buffering agent and comprising a therapeutically-effective amount of a nonsteroidal medicament and/or a corticosteroid: The transdermal formulation is administered before symptoms of inflammation are experienced by the subject. The transdermal formulation prevents inflammation and/or reduces the severity of upcoming inflammation is determined in part by a reduction of blood or serum levels of C-Reactive Protein relative to a subject that is not administered the combination therapy.

In an addition aspect, the present disclosure provides a transdermal formulation for use in method of reducing the likelihood a recurrent inflammation, the method comprising administering to a subject that previously has been treated for inflammation, the transdermal formulation comprising a therapeutically-effective amount of a buffering agent, and comprising a therapeutically-effective amount of a nonsteroidal medicament and/or a corticosteroid The transdermal formulation is administered before symptoms of inflammation are experienced by the subject and reduces the likelihood of a recurrent inflammation is determined in part by a reduction of blood or serum levels of C-Reactive Protein relative to a subject that is not administered the combination therapy.

In yet an additional, the present disclosure provides a transdermal formulation for use in method of treating chronic inflammation, the method comprising administering to a subject that previously has been treated for inflammation, the transdermal formulation comprising a therapeutically-effective amount of a buffering agent and comprising a therapeutically-effective amount of a nonsteroidal medicament and/or a corticosteroid. Here, treating chronic inflammation is determined in part by a reduction of blood or serum levels of C-Reactive Protein relative to a subject that is not administered the combination therapy.

In yet a further aspect, the present disclosure provides a transdermal formulation for use in method of treating a bone density disorder in a subject in need thereof. The method comprising administering to the subject, the transdermal formulation comprising a therapeutically-effective amount of a buffering agent.

In embodiments, the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

In any aspect or embodiment, the buffering agent is selected from Sodium Hydroxide (Sodium oxidanide), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof. Preferably, the buffering agent is sodium bicarbonate and/or sodium carbonate.

The concentration of the buffering agent in a transdermal formulation may be from about 10% to about 50% w/w of the transdermal formulation, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%. The concentration of the buffering agent in a transdermal formulation may be at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, or at least 50%. The concentration of the buffering agent in a transdermal formulation may be no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50%. The concentration of the buffering agent in a transdermal formulation may be from about 11% to about 15%, from about 12% to about 16%, from about 13% to about 17%, from about 14% to about 18%, from about 15% to about 19%, from about 16% to about 20%, from about 17% to about 21%, from about 18% to about 22%, from about 19% to about 23%, from about 20% to about 24%, from about 21% to about 25%, from about 22% to about 26%, from about 23% to about 27%, from about 24% to about 28%, from about 25% to about 29%, from about 26% to about 30%, from about 27% to about 31%, from about 28% to about 32%, from about 29% to about 33%, from about 30% to about 34%, from about 31% to about 35%, from about 32% to about 36%, from about 33% to about 37%, from about 34% to about 38%, from about 35% to about 39%, from about 36% to about 40%, from about 37% to about 41%, from about 38% to about 42%, from about 39% to about 43%, from about 40% to about 44%, from about 41% to about 45%, from about 42% to about 46%, from about 43% to about 47%, from about 44% to about 48%, from about 45% to about 49%, or from about 46% to about 50% and any range therebetween.

The buffering agent, e.g., sodium bicarbonate or sodium carbonate, in a transdermal formulation may be at a concentration from about 30% w/w to about 35% w/w. The buffering agent, e.g., sodium bicarbonate or sodium carbonate, may be at a concentration of about 30%, about 30.1%, about 30.2%, about 30.3%, about 30.4%, about 30.5%, about 30.6%, about 30.7%, about 30.8%, about 30.9%, about 31%, about 31.1%, about 31.2%, about 31.3%, about 31.4%, about 31.5%, about 31.6%, about 31.7%, about 31.8%, about 31.9%, about 32%, about 32.1%, about 32.2%, about 32.3%, about 32.4%, about 32.5%, about 32.6%, about 32.7%, about 32.8%, about 32.9%, about 33%, about 33.1%, about 33.2%, about 33.3%, about 33.4%, about 33.5%, about 33.6%, about 33.7%, about 33.8%, about 33.9%, about 34%, about 34.1%, about 34.2%, about 34.3%, about 34.4%, about 34.5%, about 34.6%, about 34.7%, about 34.8%, about 34.9%, or about 35%. The buffering agent, e.g., sodium bicarbonate or sodium carbonate, may be at a concentration of at least 30%, at least 30.1%, at least 30.2%, at least 30.3%, at least 30.4%, at least 30.5%, at least 30.6%, at least 30.7%, at least 30.8%, at least 30.9%, at least 31%, at least 31.1%, at least 31.2%, at least 31.3%, at least 31.4%, at least 31.5%, at least 31.6%, at least 31.7%, at least 31.8%, at least 31.9%, at least 32%, at least 32.1%, at least 32.2%, at least 32.3%, at least 32.4%, at least 32.5%, at least 32.6%, at least 32.7%, at least 32.8%, at least 32.9%, at least 33%, at least 33.1%, at least 33.2%, at least 33.3%, at least 33.4%, at least 33.5%, at least 33.6%, at least 33.7%, at least 33.8%, at least 33.9%, at least 34%, at least 34.1%, at least 34.2%, at least 34.3%, at least 34.4%, at least 34.5%, at least 34.6%, at least 34.7%, at least 34.8%, at least 34.9%, or at least 35%. The buffering agent, e.g., sodium bicarbonate or sodium carbonate, may be at a concentration of no more than 30%, no more than 30.1%, no more than 30.2%, no more than 30.3%, no more than 30.4%, no more than 30.5%, no more than 30.6%, no more than 30.7%, no more than 30.8%, no more than 30.9%, no more than 31%, no more than 31.1%, no more than 31.2%, no more than 31.3%, no more than 31.4%, no more than 31.5%, no more than 31.6%, no more than 31.7%, no more than 31.8%, no more than 31.9%, no more than 32%, no more than 32.1%, no more than 32.2%, no more than 32.3%, no more than 32.4%, no more than 32.5%, no more than 32.6%, no more than 32.7%, no more than 32.8%, no more than 32.9%, no more than 33%, no more than 33.1%, no more than 33.2%, no more than 33.3%, no more than 33.4%, no more than 33.5%, no more than 33.6%, no more than 33.7%, no more than 33.8%, no more than 33.9%, no more than 34%, no more than 34.1%, no more than 34.2%, no more than 34.3%, no more than 34.4%, no more than 34.5%, no more than 34.6%, no more than 34.7%, no more than 34.8%, no more than 34.9%, or no more than 35%. The buffering agent, e.g., sodium bicarbonate or sodium carbonate, may be at a concentration of from about 30% to about 31%, from about 30.1% to about 31.1%, from about 30.2% to about 31.2%, from about 30.3% to about 31.3%, from about 30.4% to about 31.4%, from about 30.5% to about 31.5%, from about 30.6% to about 31.6%, from about 30.7% to about 31.7%, from about 30.8% to about 31.8%, from about 30.9% to about 31.9%, from about 31% to about 32%, from about 31.1% to about 32.1%, from about 31.2% to about 32.2%, from about 31.3% to about 32.3%, from about 31.4% to about 32.4%, from about 31.5% to about 32.5%, from about 31.6% to about 32.6%, from about 31.7% to about 32.7%, from about 31.8% to about 32.8%, from about 31.9% to about 32.9%, from about 32% to about 33%, from about 32.1% to about 33.1%, from about 32.2% to about 33.2%, from about 32.3% to about 33.3%, from about 32.4% to about 33.4%, from about 32.5% to about 33.5%, from about 32.6% to about 33.6%, from about 32.7% to about 33.7%, from about 32.8% to about 33.8%, from about 32.9% to about 33.9%, from about 33% to about 34%, from about 33.1% to about 34.1%, from about 33.2% to about 34.2%, from about 33.3% to about 34.3%, from about 33.4% to about 34.4%, from about 33.5% to about 34.5%, from about 33.6% to about 34.6%, from about 33.7% to about 34.7%, from about 33.8% to about 34.8%, from about 33.9% to about 34.9%, or from about 34% to about 35%.

In any aspect or embodiment, sodium bicarbonate and/or sodium carbonate can be replaced with an alternate buffering agent, such as Sodium Hydroxide (Sodium oxidanide), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

In embodiments, the transdermal formulation comprises about 33% w/w sodium bicarbonate or sodium carbonate and about 0.5% w/w menthol. Menthol may act as either or both as a penetration enhancer and as an analgesic.

In some embodiments, the transdermal formulation has a pH from about 9 to about 11 or the transdermal formulation has a pH from about 7 to about 10.5.

In embodiments, the transdermal formulation is formulated as a cream, lotion, or ointment.

Table 1 provides a generic transdermal formulation of the present disclosure.

TABLE 1

| Ingredient | Weight (%) |
|---|---|
| Phospholipid | 5-15% |
| Emollient/moisturizer | 10-20% |
| Fatty acid | 0.5-2% |
| Alcohol | 0.5-2% |
| Oil | 1-10% |
| Surfactant | 0.5-2% |
| Deionized Water | 25-75% |
| Dextrose | 0-2% |
| Buffering Agent | 10-50% |
| Total | 100.00% |

In transdermal formulations that comprise an additional ingredient (e.g., menthol, colchicine, nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase), the amount of water may be reduced accordingly.

Table 2 provides ranges for ingredients—when present—which may be in a transdermal formulation of the present disclosure. In some cases, a formulation lacks one or more of the following ingredients and will have a weight % of zero.

TABLE 2

| Ingredient | Weight (%) |
|---|---|
| Almond Oil | 2%-4% |
| Benzyl Alcohol | 0.9%-2% |
| Buffering Agent | 30%-33% |
| Cetyl Alcohol | 2%-3% |
| Deionized Water | 25%-75% |
| Dextrose | 0.2%-2% |
| Ethanol | 1%-2% |
| Isopropyl Palmitate | 8%-15% |
| Lecithin | 3%-7% |
| Linoleic Acid | 1%-3% |
| Menthol | 0.1%-5% |
| Oleic Acid | 0.5%-2% |
| Phosphatidylcholine e.g., Phospholipon 90G) | 3%-9% |
| Poloxamer 407 | 5%-10% |
| Polyglyceryl-4 Laurate | 0.5%-2% |
| Propylene Glycol | 3%-7% |
| Safflower Oil | 1%-3% |
| Stearic Acid | 0.2%-1% |
| Stearic Alcohol | 0.4%-0.8% |
| Total | 100.00% |

In transdermal formulations that comprise an additional ingredient (e.g., menthol, colchicine, nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase), the amount of water may be reduced accordingly.

Table 3 provides an illustrative transdermal formulation of the present disclosure.

TABLE 3

| Ingredient | Weight (%) |
|---|---|
| Phosphatidylcholine (e.g., Phospholipon 90G) | 7.02% |
| Isopropyl Palmitate | 12.21% |
| Stearic Acid | 0.57% |
| Benzyl Alcohol | 1.28% |
| Safflower oil | 2.69% |
| Oleic Acid | 0.89% |
| Polyglyceryl-4 Laurate | 1.00% |
| Deionized Water | 32.85%-35.85% |
| Buffering Agent | 30%-33% |
| Poloxamer 407 | 8.49% |

Table 4 provides another illustrative transdermal formulation of the present disclosure.

TABLE 4

| Ingredient | Weight (%) |
|---|---|
| Phosphatidylcholine (e.g., Phospholipon 90G) | 7.02% |
| Isopropyl Palmitate | 12.21% |
| Stearic Acid | 0.57% |
| Benzyl Alcohol | 1.28% |
| Safflower oil | 2.69% |
| Oleic Acid | 0.89% |
| Polyglyceryl-4 Laurate | 1.00% |
| Deionized Water | 27.85%-35.75% |
| Menthol | 0.1%-5% |
| Buffering Agent | 30%-33% |
| Poloxamer 407 | 8.49% |

Table 5 provides a further illustrative transdermal formulation of the present disclosure.

TABLE 5

| Ingredient | Weight (%) |
|---|---|
| Phosphatidylcholine (e.g., Phospholipon 90G) | 7.02% |
| Isopropyl Palmitate | 12.21% |
| Stearic Acid | 0.57% |
| Benzyl Alcohol | 1.28% |
| Safflower oil | 2.69% |
| Oleic Acid | 0.89% |
| Polyglyceryl-4 Laurate | 1.00% |
| Deionized Water | 32.85% |
| Buffering Agent (e.g., sodium bicarbonate or | 33% |
| sodium carbonate) | |
| Poloxamer 407 | 8.49% |

Table 6 provides yet another illustrative transdermal formulation of the present disclosure.

TABLE 6

| Ingredient | Weight (%) |
|---|---|
| Phosphatidylcholine (e.g., Phospholipon 90G) | 7.02% |
| Isopropyl Palmitate | 12.21% |
| Stearic Acid | 0.57% |
| Benzyl Alcohol | 1.28% |
| Safflower oil | 2.69% |
| Oleic Acid | 0.89% |
| Polyglyceryl-4 Laurate | 1.00% |
| Deionized Water | 32.35% |
| Menthol | 0.5% |
| Buffering Agent (e.g., sodium bicarbonate or | 33% |
| sodium carbonate) | |
| Poloxamer 407 | 8.49% |

Table 7 provides an illustrative transdermal formulation of the present disclosure.

TABLE 7

| Ingredient | Weight (%) |
|---|---|
| Phosphatidylcholine (e.g., Phospholipon 90G) | 7.57% |
| Isopropyl Palmitate | 13.17% |
| Benzyl Alcohol | 1.38% |
| Stearic Alcohol | 0.61% |
| Safflower Oil | 2.90% |
| Oleic Acid | 0.96% |
| Polyglyceryl-4 Laurate | 1.00% |
| Deionized Water | 33.92%-36.92% |
| Buffering Agent | 30%-33% |
| Poloxamer 407 | 8.49% |

Table 8 provides another illustrative transdermal formulation of the present disclosure.

TABLE 8

| Ingredient | Weight (%) |
|---|---|
| Phosphatidylcholine (e.g., Phospholipon 90G) | 7.57% |
| Isopropyl Palmitate | 13.17% |
| Benzyl Alcohol | 1.38% |
| Stearic Alcohol | 0.61% |
| Safflower Oil | 2.90% |
| Oleic Acid | 0.96% |
| Polyglyceryl-4 Laurate | 1.00% |
| Deionized Water | 33.52%-31.92% |
| Menthol | 0.1%-5% |
| Buffering Agent | 30%-33% |
| Poloxamer 407 | 8.49% |

Table 9 provides a further illustrative transdermal formulation of the present disclosure.

TABLE 9

| Ingredient | Weight (%) |
|---|---|
| Phosphatidylcholine (e.g., Phospholipon 90G) | 7.57% |
| Isopropyl Palmitate | 13.17% |
| Benzyl Alcohol | 1.38% |
| Stearic Alcohol | 0.61% |
| Safflower Oil | 2.90% |
| Oleic Acid | 0.96% |
| Polyglyceryl-4 Laurate | 1.00% |
| Deionized Water | 31% |
| Buffering Agent (e.g., sodium bicarbonate or | 33% |
| sodium carbonate) | |
| Poloxamer 407 | 8.49% |

Table 10 provides yet another illustrative transdermal formulation of the present disclosure.

TABLE 10

| Ingredient | Weight (%) |
|---|---|
| Phosphatidylcholine (e.g., Phospholipon 90G) | 7.57% |
| Isopropyl Palmitate | 13.17% |
| Benzyl Alcohol | 1.38% |
| Stearic Alcohol | 0.61% |
| Safflower Oil | 2.90% |
| Oleic Acid | 0.96% |
| Polyglyceryl-4 Laurate | 1.00% |
| Deionized Water | 30.5% |
| Menthol | 0.5% |
| Buffering Agent (e.g., sodium bicarbonate or | 33% |
| sodium carbonate) | |
| Poloxamer 407 | 8.49% |

Table 11 provides an illustrative transdermal formulation of the present disclosure.

TABLE 11

| Ingredient | Weight (%) |
|---|---|
| Phosphatidylcholine (e.g., Phospholipon 90G) | 4.09% |
| Benzyl Alcohol | 1.71% |
| Isopropyl Palmitate | 7.11% |
| Stearic Acid | 0.33% |
| Cetyl Alcohol | 2.00% |
| Ethanol | 1.50% |
| Safflower Oil | 1.58% |
| Oleic Acid | 0.51% |
| Almond Oil | 3.00% |
| Propylene Glycol | 5.00% |
| Polyglyceryl 4-Laurate | 1.00% |
| Deionized Water | 33.68% |
| Poloxamer 407 | 5.49% |
| Sodium Bicarbonate | 33.00% |

Table 12 provides another illustrative transdermal formulation of the present disclosure.

TABLE 12

| Ingredient | Weight (%) |
|---|---|
| Phosphatidylcholine (e.g., Phospholipon 90G) | 5.20% |
| Isopropyl Palmitate | 9.05% |
| Benzyl Alcohol | 0.95% |
| Stearic Acid | 0.42% |
| Safflower Oil | 1.99% |
| Oleic Acid | 0.66% |
| Polyglyceryl 4-Laurate | 1.00% |
| Deionized Water | 41.42% |
| Poloxamer 407 | 6.30% |
| Sodium Bicarbonate | 33.00% |

Table 13 provides a further illustrative transdermal formulation of the present disclosure.

TABLE 13

| Ingredient | Weight (%) |
|---|---|
| Phosphatidylcholine (e.g., Phospholipon 90G) | 6.62% |
| Isopropyl Palmitate | 11.53% |
| Benzyl Alcohol | 1.21% |
| Stearic Acid | 0.54% |
| Safflower Oil | 2.54% |
| Oleic Acid | 0.84% |
| Polyglyceryl 4-Laurate | 1.00% |
| Deionized Water | 52.72% |
| Poloxamer 407 | 8.01% |
| Sodium Bicarbonate | 15.00% |

Table 14 provides yet another illustrative transdermal formulation of the present disclosure.

TABLE 14

| Ingredient | Weight (%) |
|---|---|
| Phosphatidylcholine (e.g., Phospholipon 90G) | 5.20% |
| Isopropyl Palmitate | 9.05% |
| Benzyl Alcohol | 0.95% |
| Stearic Acid | 0.42% |
| Safflower Oil | 1.99% |
| Oleic Acid | 0.66% |
| Polyglyceryl 4-Laurate | 1.00% |
| Deionized Water | 41.42% |
| Poloxamer 407 | 6.30% |
| Tris | 33.00% |

Table 15 provides an illustrative transdermal formulation of the present disclosure.

TABLE 15

| Ingredient | Weight (%) |
|---|---|
| Phosphatidylcholine (e.g., Phospholipon 90G) | 5.20% |
| Isopropyl Palmitate | 9.05% |
| Benzyl Alcohol | 0.95% |
| Stearic Acid | 0.42% |
| Safflower Oil | 1.99% |
| Oleic Acid | 0.66% |
| Polyglyceryl 4-Laurate | 1.00% |
| Deionized Water | 41.42% |
| Poloxamer 407 | 6.30% |
| Calcium Carbonate | 33.00% |

Table 16 provides another illustrative transdermal formulation of the present disclosure.

TABLE 16

| Ingredient | Weight (%) |
|---|---|
| Phosphatidylcholine (e.g., Phospholipon 90G) | 5.20% |
| Isopropyl Palmitate | 9.05% |
| Benzyl Alcohol | 0.95% |
| Stearic Acid | 0.42% |
| Safflower Oil | 1.99% |
| Oleic Acid | 0.66% |
| Polyglyceryl 4-Laurate | 1.00% |
| Deionized Water | 41.42% |
| Potassium phosphate dibasic | 33.00% |
| Poloxamer 407 | 6.30% |

Table 17 provides another illustrative transdermal formulation of the present disclosure.

TABLE 17

| Ingredient | Weight (%) |
|---|---|
| Phosphatidylcholine (e.g., Phospholipon 90G) | 4.03% |
| Benzyl Alcohol | 1.68% |
| Isopropyl Palmitate | 7.00% |
| Stearic Acid | 0.32% |
| Cetyl Alcohol | 2.00% |
| Menthol | 0.50% |
| Ethanol | 1.50% |
| Safflower Oil | 1.55% |
| Oleic Acid | 0.50% |
| Almond Oil | 3.00% |
| Propylene Glycol | 5.00% |
| Deionized Water | 33.16% |
| Dextrose (Anhydrous) | 0.35% |
| Poloxamer 407 | 5.40% |
| Polyglyceryl-4 laurate | 1.00% |
| Sodium Bicarbonate | 33.00% |

Table 18 provides another illustrative transdermal formulation of the present disclosure.

TABLE 18

| Ingredient | Weight (%) |
|---|---|
| Deionized Water | 41.31% |
| Dextrose (Anhydrous) | 0.45% |
| Phosphatidylcholine | 5.19% |
| Isopropyl Palmitate | 9.02% |
| Benzyl Alcohol | 0.94% |
| Stearic Acid | 0.52% |
| Linoleic Acid | 1.76% |
| Oleic Acid | 0.79% |
| Polyglyceryl-4 Laurate | 0.72% |
| Poloxamer 407 | 6.28% |
| Buffering Agent | 33.00% |

Table 19 provides another illustrative transdermal formulation of the present disclosure.

TABLE 19

| Ingredient | Weight (%) |
|---|---|
| Menthol | 0.50% |
| Ethanol | 1.50% |
| Benzyl Alcohol | 1.50% |
| Cetyl Alcohol | 2.00% |
| Almond Oil | 3.00% |
| Lecithin | 7.00% |
| Isopropyl Palmitate | 7.00% |
| Propylene Glycol | 5.00% |
| Poloxamer | 5.40% |
| Deionized Water | 33.10% |
| Buffering agent | 33.00% |
| Polyglyceryl-4 Laurate | 1.00% |

A transdermal formulation of the present disclosure, e.g., as disclosed in Table 1 to Table 19, may comprise nine or more, ten or more, eleven or more, twelve or more, thirteen or more, fourteen or more, fifteen or more, sixteen or more, seventeen or more, or eighteen of the following ingredients: Almond Oil, Benzyl Alcohol, Buffering Agent, Cetyl Alcohol, Deionized Water, Dextrose, Ethanol, Isopropyl Palmitate, Lecithin, Linoleic Acid, Menthol, Oleic Acid, Phosphatidylcholine, Poloxamer 407, Polyglyceryl-4 Laurate, Propylene Glycol, Safflower Oil, Stearic Acid, and Stearic Alcohol. The buffering agent may be Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

A transdermal formulation of the present disclosure, e.g., as disclosed in Table 1 to Table 19, may comprise nine or more, ten or more, eleven or more, twelve or more, thirteen or more, fourteen or more, fifteen or more, sixteen or more, seventeen or more or eighteen of the following ingredients and in the following amounts: Almond Oil from about 2% to about 4%; Benzyl Alcohol from about 0.9% to about 2%; Buffering Agent from about 30% to about 33%; Cetyl Alcohol from about 2% to about 3%; Deionized Water from about 25% to about 75%; Dextrose from about 0.2% to about 2%; Ethanol from about 1% to about 2%; Isopropyl Palmitate from about 8% to about 15%; lecithin from about 5% to about 10%; linoleic Acid from about 1% to about 3%; Menthol from about 0.1% to about 5%; Oleic Acid from about 0.5% to about 2%; Phosphatidylcholine from about 3% to about 9%; Poloxamer 407 from about 5% to about 10%; Polyglyceryl-4 Laurate from about 0.5% to about 2%; Propylene Glycol from about from 3% to about 7%; Safflower Oil from about from 1% to about 3%; Stearic Acid from about 0.2% to about 1%; and Stearic Alcohol from about 0.4% to about 0.8%. The buffering agent may be Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

An illustrative transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

In transdermal formulations that comprise an additional ingredient (e.g., menthol, colchicine, nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase), the amount of water may be reduced accordingly. Thus, the amounts of water in the illustrative formulations of Tables 2 to Table 19 will have a reduced amount of water. As an example, colchicine may be included in a transdermal formulation in an amount from 0.02% to about 0.4% w/w, e.g., about 0.03% to about 0.36% w/w.

In various embodiments, the amount of buffering agent in the formulation is at least about 5% (w/w) to about 40% (w/w). In some embodiments, the amount of buffering agent in the formulation is at least about 10% (w/w) to about 40% (w/w). In some embodiments, the amount of buffering agent in the formulation is at least about 15% (w/w) to about 40% (w/w). In some embodiments, the amount of buffering agent in the formulation is at least about 20% (w/w) to about 40% (w/w). In some embodiments, the amount of buffering agent in the formulation is at least about 25% (w/w) to about 40% (w/w). In various embodiments, the amount of buffering agent in the formulation is at least about 5% (w/w) to about 35% (w/w). In various embodiments, the amount of buffering agent in the formulation is at least about 10% (w/w) to about 35% (w/w). In various embodiments, the amount of buffering agent in the formulation is at least about 15% (w/w) to about 35% (w/w). In various embodiments, the amount of buffering agent in the formulation is at least about 20% (w/w) to about 35% (w/w). In various embodiments, the amount of buffering agent in the formulation is at least about 25% (w/w) to about 35% (w/w).

In various embodiments, the amount of buffering agent in the formulation is at least 1% (w/w). In some embodiments, the amount of buffering agent in the formulation is at least 5% (w/w). In some embodiments, the amount of buffering agent in the formulation is at least 10% (w/w). In some embodiments, the amount of buffering agent in the formulation is at least 15% (w/w). In some embodiments, the amount of buffering agent in the formulation is at least 20% (w/w).

In various embodiments, the amount of buffering agent in the formulation is less than 40% (w/w). In various embodiments, the amount of buffering agent in the formulation is less than 35% (w/w). In various embodiments, the amount of buffering agent in the formulation is less than 30% (w/w). In various embodiments, the amount of buffering agent in the formulation is less than 25% (w/w). In various embodiments, the amount of buffering agent in the formulation is less than 20% (w/w). In various embodiments, the amount of buffering agent in the formulation is less than 15% (w/w). In various embodiments, the amount of buffering agent in the formulation is less than 10% (w/w).

In some cases, a combination of buffering agents is used. In some embodiments, the formulation comprises two buffering agents in equal amount, e.g., 15% sodium bicarbonate and 15% calcium carbonate or 5% Lysine and 5% IEPA. In other cases, the formulation comprises two buffering agents in differing amounts, e.g., 10% Tris and 15% magnesium carbonate or 7% Arginine and 3% glycine. In further cases, the formulation comprises three buffering agents in equal amounts, e.g., 5% sodium bicarbonate, 5% calcium carbonate, and 5% magnesium carbonate. In various cases, the formulation comprises three buffering agents in different amounts e.g., 5% IEPA, 3% sodium carbonate, and 2% monosodium phosphate or 7% tripotassium phosphate, 8% arginine, and 9% calcium carbonate. In other cases, the formulation comprises two or more buffering agents in equal amount. In various cases, the formulation comprises two or more buffering agents in different amount.

In various embodiments, the amount of the combination of buffering agents in the formulation is at least about 5% (w/w) to about 40% (w/w). In some embodiments, the amount of the combination of buffering agents in the formulation is at least about 10% (w/w) to about 40% (w/w). In some embodiments, the amount of the combination of buffering agents in the formulation is at least about 15% (w/w) to about 40% (w/w). In some embodiments, the amount of the combination of buffering agents in the formulation is at least about 20% (w/w) to about 40% (w/w). In some embodiments, the amount of the combination of buffering agents in the formulation is at least about 25% (w/w) to about 40% (w/w). In various embodiments, the amount of the combination of buffering agents in the formulation is at least about 5% (w/w) to about 35% (w/w). In various embodiments, the amount of the combination of buffering agents in the formulation is at least about 10% (w/w) to about 35% (w/w). In various embodiments, the amount of the combination of buffering agents in the formulation is at least about 15% (w/w) to about 35% (w/w). In various embodiments, the amount of the combination of buffering agents in the formulation is at least about 20% (w/w) to about 35% (w/w). In various embodiments, the amount of the combination of buffering agents in the formulation is at least about 25% (w/w) to about 35% (w/w).

In various embodiments, the amount of the combination of buffering agents in the formulation is at least 1% (w/w). In some embodiments, the amount of the combination of buffering agents in the formulation is at least 5% (w/w). In some embodiments, the amount of the combination of buffering agents in the formulation is at least 10% (w/w). In some embodiments, the amount of the combination of buffering agents in the formulation is at least 15% (w/w). In some embodiments, the amount of the combination of buffering agents in the formulation is at least 20% (w/w).

In various embodiments, the amount of the combination of buffering agents in the formulation is less than 40% (w/w). In various embodiments, the amount of the combination of buffering agents in the formulation is less than 35% (w/w). In various embodiments, the amount of the combination of buffering agents in the formulation is less than 30% (w/w). In various embodiments, the amount of the combination of buffering agents in the formulation is less than 25% (w/w). In various embodiments, the amount of the combination of buffering agents in the formulation is less than 20% (w/w). In various embodiments, the amount of the combination of buffering agents in the formulation is less than 15% (w/w). In various embodiments, the amount of the combination of buffering agents in the formulation is less than 10% (w/w).

In embodiments, the sodium bicarbonate or sodium carbonate is at a concentration of about 33% w/w of the transdermal formulation.

In some embodiments, the transdermal formulation comprises menthol. In various embodiments, the menthol is at a concentration from about 0.1% to about 5.0% w/w of the transdermal formulation. Menthol may act as either or both as a penetration enhancer and as an analgesic.

Menthol may be present in a transdermal formulation in an amount from about 0.1% to about 1.0% (w/w), e.g., about 0.1%, 0.2%, 0.3%, 0.4%, 0.5%, 0.6%, 0.7%, 0.8%, 0.9%, or 1.0% or any percentage therebetween. As examples, about 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, or 1.00%, or any percentage therebetween. The ranges may be from about 0.1% to about 0.2%, from about 0.1% to about 0.3%, from about 0.1% to about 0.5%, from about 0.1% to about 0.7%, from about 0.1% to about 0.8%, from about 0.2% to about 0.3%, from about 0.2% to about 0.4%, from about 0.2% to about 0.6%, from about 0.2% to about 0.8%, from about 0.2% to about 0.9%, from about 0.2% to about 0.5%, from about 0.2% to about 0.7%, from about 0.3% to about 0.4%, from about 0.3% to about 0.5%, from about 0.3% to about 0.7%, from about 0.3% to about 0.9%, from about 0.3% to about 1.0% from about 0.3% to about 0.6%, from about 0.3% to about 0.8%, from about 0.4% to about 0.5%, from about 0.4% to about 0.6%, from about 0.4% to about 0.8%, from about 0.4% to about 1.0%, from about 0.4% to about 0.7%, from about 0.4% to about 0.9%, from about 0.5% to about 0.6%, from about 0.5% to about 0.7%, from about 0.5% to about 0.9%, from about 0.5% to about 0.8%, from about 0.5% to about 1.0%, from about 0.6% to about 0.7%, from about 0.6% to about 0.8%, from about 0.6% to about 1.0%%, from about 0.6% to about 0.9%, from about 0.7% to about 0.8%, from about 0.7% to about 0.9%, from about 0.7% to about 1.0%, from about 0.8% to about 0.9%, from about 0.8% to about 1.0%, or from about 0.9% to about 1.0%, and any subrange therebetween. Menthol may be present in a transdermal formulation in an amount from about 1% to about 10% (w/w), e.g., about 1%, 2%, 3%, 4%, 5% or any percentage therebetween. As examples, about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or any percentage therebetween. The ranges may be from about 1% to about 2%, from about 1% to about 3%, from about 1% to about 4%, from about 1% to about 5%, from about 2% to about 3%, from about 2% to about 4%, from about 2% to about 5%, from about 3% to about 4%, from about 3% to about 5%, from about 4% to about 5%, and any subrange therebetween.

In embodiments of the any herein-disclosed aspects, the transdermal formulation comprises a penetrant or penetration enhancer. In some embodiments, the penetrant or penetration enhancer comprises one or more of phosphatidylcholine (e.g., Phospholipon® 90 G), isopropyl palmitate (IPP), stearic acid, benzyl alcohol, safflower oil, almond oil, oleic acid, polyglyceryl-4 laurate, poloxamer 407, poloxamer 188, poloxamer 124, menthol, propylene glycol, cetyl alcohol, ethanol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin. In various embodiments, the penetrant or penetration enhancer comprises one or more of phosphatidylcholine (e.g., Phospholipon® 90 G), isopropyl palmitate (IPP), stearic acid, benzyl alcohol, polyglyceryl-4 laurate, poloxamer 407 poloxamer 188, or poloxamer 124. In embodiments, the penetrant or penetration enhancer comprises phosphatidylcholine, hydrogenated phosphatidylcholine, phosphatidylserine, phosphatidylethanolamine, phosphatidylinositol, one or more phosphatides, or one or more inositol phosphatides. In some embodiments, the penetrant or penetration enhancer comprises from about 3 to about 15% w/w phosphatidylcholine, from about 5 to about 20% w/w isopropyl palmitate, from about 0.2% to about 1% w/w stearic acid, about 1% w/w benzyl alcohol, from about 1 to about 10% w/w polyglyceryl-4 laurate and from about 5 to about 20% w/w poloxamer 407. In various embodiments, the penetrant or penetration enhancer comprises benzyl alcohol and/or wherein the penetrant or penetration enhancer comprises a synthetic lecithin.

The concentration of phosphatidylcholine in a transdermal delivery formulation may be from about 3% to about 9%. In some cases, the concentration of phosphatidylcholine in a transdermal delivery formulation is at least 3%, at least 4%, at least 5%, at least 6%, at least 7%, at least 8%, at least 9%, at least 10%, at least 11%, at least 12%, at least 13%, at least 14%, at least 15% or more. The concentration of phosphatidylcholine in a transdermal delivery formulation may be not more than 3%, not more than 4%, not more than 5%, not more than 6%, not more than 7%, not more than 8%, not more than 9%, not more than 10%, not more than 11%, not more than 12%, not more than 13%, not more than 14%, not more than 15% or more. The concentration of phosphatidylcholine in a transdermal delivery formulation may be about 3%, about 4%, about 5%, about 6%, about 7%, about 8%, about 9%, about 10%, about 11%, about 12%, about 13%, about 14%, about 15%. The concentration of Phosphatidylcholine in a transdermal delivery formulation may be from 3% to 5%, from 4% to 6%, from 5% to 7%, from 6% to 8%, from 7% to 9%, from 8% to 10%, from 9% to 11%, from 10% to 12%, from 11% to 13%, from 12% to 14%, from 13% to 15%, and any range therebetween. An example of a phosphatidylcholine useful in formulations of the present disclosure is Phospholipon® 90 G.

The concentration of benzyl alcohol in a transdermal formulation may be from about 0.9% to about 2%. In some cases, the concentration of benzyl alcohol in a transdermal formulation is at least 0.25%, at least 0.5%, at least 0.75%, at least 1%, at least 2%, at least 2.5%, at least 3%, at least 4%, at least 5% or more. The concentration of benzyl alcohol in a transdermal formulation may be about 0.25%, about 0.5%, about 0.75%, about 1%, about 2%, about 2.5%, about 3%, about 4%, about 5% or more. The concentration of benzyl alcohol in a transdermal formulation may be from 0.25% to 5%; from 0.5% to 4%, from 0.75% to 3%, from 1% to 2.5% or from 0.5% to 2%. The concentration of benzyl alcohol in a transdermal formulation may be no more than 0.25%, no more than 0.5%, no more than 0.75%, no more than 1%, no more than 2%, no more than 2.5%, no more than 3%, no more than 4%, no more than 5%.

The concentration of Polyglyceryl-4 Laurate in a transdermal delivery formulation may from about 0.5% to about 2%. In some cases, the concentration of Polyglyceryl-4 Laurate in a transdermal delivery formulation may be at least 0.25%, at least 0.5%, at least 0.75%, at least 1%, at least 2%, at least 2.5% or more. The concentration of Polyglyceryl-4 Laurate in a transdermal delivery formulation may be about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, or more. The concentration of Polyglyceryl-4 Laurate in a transdermal delivery formulation may be no more than 0.1%, no more than 0.2%, no more than 0.3%, no more than 0.4%, no more than 0.5%, no more than 0.6%, no more than 0.7%, no more than 0.8%, no more than 0.9%, no more than 1.0%, no more than 1.1%, no more than 1.2%, no more than 1.3%, no more than 1.4%, no more than 1.5%. The concentration of Polyglyceryl-4 Laurate in a transdermal delivery formulation may be from 0.1% to 1.0%, from 0.2% to 6%, from 0.3% to 0.5%, from 0.3% to 0.6%, from 0.4% to 0.7%, from 0.5% to 0.6%, from 0.5% to 0.8%, from 0.6 to 0.9%, from 0.7% to 1.0%, from 0.8 to 1.1% or from 0.9% to 1.2%, from 1.0 to 1.3% or from 1.1% to 1.4%, or from 1.2 to 1.5% and any range therebetween.

In embodiments, the transdermal formulation comprises a source of fatty acids. In some embodiments, the source of fatty acids comprises one or more of an alkanoic acid, almond oil, caprid acid, diacid, ethyloctadecanoic acid, hexanoic acid, lactic acid, lauric acid, a lecithin, linoelaidic acid, linoleic acid, linolenic acid, neodecanoic acid, oleic acid, palmitic acid, pelargonic acid, propionic acid, safflower oil, almond oil, stearic acid, and vaccenic acid, wherein the lecithin is selected from an egg lecithin, a soy lecithin, and a synthetic lecithin.

The concentration of safflower oil in a transdermal delivery formulation may be from about 1% to about 5%. The concentration of safflower oil in a transdermal delivery formulation may be at least 1%, at least 2%, at least 2.5%, at least 3%, at least 4%, at least 5% or more. The concentration of safflower oil in a transdermal delivery formulation may be about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, or any amount therebetween. The concentration of safflower oil in a transdermal delivery formulation may be from 1% to 5%; from 1.25% to 4%, from 1.5% to 3%, from 1.7% to 2.5% or from 1.6% to 2%. The concentration of safflower oil in a transdermal delivery formulation may be no more than 1%, no more than 2%, no more than 2.5%, no more than 3%, no more than 4%, no more than 5%.

The concentration of oleic acid in a transdermal delivery formulation may be from about 0.5% to about 2%. The concentration of oleic acid in a transdermal delivery formulation may be at least 0.25%, at least 0.5%, at least 0.75%, at least 1%, at least 2%, at least 2.5% or more. The concentration of oleic acid in a transdermal delivery formulation may be about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 1.0% or more. The concentration of oleic acid in a transdermal delivery formulation may be no more than 0.1%, no more than 0.2%, no more than 0.3%, no more than 0.4%, no more than 0.5%, no more than 0.6%, no more than 0.7%, no more than 0.8%, no more than 0.9%, no more than 1.0%. The concentration of oleic acid in a transdermal delivery formulation may be from 0.1% to 1.0%, from 0.2% to 0.9%, from 0.2% to 0.3%, from 0.3% to 0.4%, from 0.3% to 0.8%, from 0.4% to 0.7%, from 0.5% to 0.8%, from 0.6 to 0.9% or from 0.7% to 1.0% and any range therebetween. The concentration of oleic acid in a transdermal delivery formulation may be from 0.5% to 0.75%, 0.75% to 1%, 1% to 2.0%, from 1.2% to 1.9%, from 1.2% to 1.3%, from 1.3% to 1.4%, from 1.3% to 1.8%, from 1.4% to 1.7%, from 1.5% to 1.8%, from 1.6 to 1.9% or from 1.7% to 2.0% and any range therebetween.

The concentration of stearic acid in a transdermal delivery formulation may be from about 0.2% to about 1%. The concentration of stearic acid in a transdermal delivery formulation may be at least 0.2%, at least 0.25%, at least 0.5%, at least 0.75%, at least 1%, at least 2%, at least 2.5% or more. The concentration of stearic acid in a transdermal delivery formulation may be about 0.1%, about 0.2%, about 0.3%, about 0.4%, about 0.5%, about 0.6%, about 0.7%, about 0.8%, about 0.9%, about 1.0% or more. The concentration of stearic acid in a transdermal delivery formulation may be no more than 0.1%, no more than 0.2%, no more than 0.3%, no more than 0.4%, no more than 0.5%, no more than 0.6%, no more than 0.7%, no more than 0.8%, no more than 0.9%, no more than 1.0%. The concentration of stearic acid in a transdermal delivery formulation may be from 0.1% to 1.0%, from 0.2% to 6%, from 0.3% to 0.5%, from 0.3% to 0.6%, from 0.4% to 0.7%, from 0.5% to 0.6%, from 0.5% to 0.8%, from 0.6 to 0.9% or from 0.7% to 1.0% and any range therebetween.

In various embodiments, the transdermal formulation comprises a polar solvent comprising one or more of ethanol, isopropyl palmitate (IPP), and water.

The concentration of isopropyl palmitate in a transdermal delivery formulation may be from about 8% to about 15%. The concentration of isopropyl palmitate in a transdermal delivery formulation may be at least 3%, at least 4%, at least 5%, at least 6%, at least 7%, at least 8%, at least 9%, at least 10%, at least 11%, at least 12%, at least 13%, at least 14%, at least 15% or more. The concentration of isopropyl palmitate in a transdermal delivery formulation may be not more than 3%, not more than 4%, not more than 5%, not more than 6%, not more than 7%, not more than 8%, not more than 9%, not more than 10%, not more than 11%, not more than 12%, not more than 13%, not more than 14%, not more than 15% or more. The concentration of isopropyl palmitate in a transdermal delivery formulation may be about 3%, about 4%, about 5%, about 6%, about 7%, about 8%, about 9%, about 10%, about 11%, about 12%, about 13%, about 14%, about 15%. The concentration of isopropyl palmitate in a transdermal delivery formulation may be from 3% to 5%, from 4% to 6%, from 5% to 7%, from 6% to 8%, from 7% to 9%, from 8% to 10%, from 9% to 11%, from 10% to 12%, from 11% to 13%, from 12% to 14%, from 13% to 15%, and any range therebetween.

The concentration of deionized water in a transdermal delivery formulation may be from about 25% to about 75%. The concentration of deionized water in a transdermal delivery formulation may be at least 25%, at least 30%, at least 40%, at least 50%, at least 60%, at least 70%, at least 80%, or more. The concentration of deionized water in a transdermal delivery formulation may be not more than 25%, not more than 30%, not more than 40%, not more than 50%, not more than 60%, not more than 70%, not more than 80%. The concentration of deionized water in a transdermal delivery formulation may be about 30%, about 40%, about 50%, about 60%, about 70%, about 80%. The concentration of deionized water in a transdermal delivery formulation may be from 30% to 50%, from 40% to 60%, from 50% to 70%, from 60% to 80%, and any range therebetween. In transdermal formulations that comprise an additional ingredient (e.g., menthol, colchicine, nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase), the amount of water may be reduced accordingly.

In embodiments, the transdermal formulation comprises one or more of a humectant, an emulsifier, a surfactant, and an emollient. In some embodiments, the emulsifier comprises one or more of cetyl alcohol, Durosoft®, and Phospholipon® 90 G. In various embodiments, the humectant comprises propylene glycol.

In embodiments, the surfactant comprises one or more of a poloxamer (e.g., poloxamer 407, poloxamer 188, and poloxamer 124), polyglyceryl-4 laurate, polyoxyethylated castor oil derivative, nonoxynol, octoxynol, phenylsulfonate, a polyoleates, Rewopal®, sodium laurate, sodium lauryl sulfate (sodium dodecyl sulfate), sodium oleate, sorbitan dilaurate, sorbitan dioleate, a sorbitan monolaurate, a sorbitan monooleate; sorbitan trilaurate, sorbitan trioleate, a sorbitan monopalmitate, a sorbitan stearate; a polyethylene glycol, a nonylphenyl ether, p-(1,1,3,3-tetramethylbutyl)-phenyl ether (Triton™ X-100), or a polysorbate (e.g., a Tween®). In some embodiments, the poloxamer is a Pluronic®.

The concentration of poloxamer 407 in a transdermal delivery formulation may be from about 5% to about 10%. The concentration of poloxamer 407 in a transdermal delivery formulation may be at least 3%, at least 4%, at least 5%, at least 6%, at least 7%, at least 8%, at least 9%, at least 10%, at least 11%, at least 12%, at least 13%, at least 14%, at least 15% or more. The concentration of poloxamer 407 in a transdermal delivery formulation may be not more than 3%, not more than 4%, not more than 5%, not more than 6%, not more than 7%, not more than 8%, not more than 9%, not more than 10%, not more than 11%, not more than 12%, not more than 13%, not more than 14%, not more than 15% or more. The concentration of poloxamer 407 in a transdermal delivery formulation may be about 3%, about 4%, about 5%, about 6%, about 7%, about 8%, about 9%, about 10%, about 11%, about 12%, about 13%, about 14%, about 15%.

The concentration of poloxamer 407 in a transdermal delivery formulation may be from 3% to 5%, from 4% to 6%, from 5% to 7%, from 6% to 8%, from 7% to 9%, from 8% to 10%, from 9% to 11%, from 10% to 12%, from 11% to 13%, from 12% to 14%, from 13% to 15%, and any range therebetween.

The concentration of Almond Oil in a transdermal delivery formulation may be from about 2% to about 4%. The concentration of Almond Oil in a transdermal delivery formulation may be about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, or any amount therebetween. The concentration of Almond Oil in a transdermal delivery formulation may be less than 2.0%, less than 2.1%, less than 2.2%, less than 2.3%, less than 2.4%, less than 2.5%, less than 2.6%, less than 2.7%, less than 2.8%, less than 2.9%, less than 3.0%, less than 3.1%, less than 3.2%, less than 3.3%, less than 3.4%, less than 3.5%, less than 3.6%, less than 3.7%, less than 3.8%, less than 3.9%, or less than 4.0%. The concentration of Almond Oil in a transdermal delivery formulation may be at least 2.0%, at least 2.1%, at least 2.2%, at least 2.3%, at least 2.4%, at least 2.5%, at least 2.6%, at least 2.7%, at least 2.8%, at least 2.9%, at least 3.0%, at least 3.1%, at least 3.2%, at least 3.3%, at least 3.4%, at least 3.5%, at least 3.6%, at least 3.7%, at least 3.8%, at least 3.9%, or at least 4.0%. The concentration of Almond Oil in a transdermal delivery formulation may from about 2.0% to about 2.5%, about 2.5% to about 3.0%, about 3.0% to about 3.5%, about 3.5% to about 4%, and any range therebetween.

The concentration of Cetyl Alcohol in a transdermal delivery formulation may be from about 2% to about 3%. The concentration of Cetyl Alcohol in a transdermal delivery formulation may be about 2.0%, about 2.1%, about 2.2%, about 2.3%, about 2.4%, about 2.5%, about 2.6%, about 2.7%, about 2.8%, about 2.9%, about 3.0% or any amount therebetween. The concentration of Cetyl Alcohol in a transdermal delivery formulation may be less than less than 2.1%, less than 2.2%, less than 2.3%, less than 2.4%, less than 2.5%, less than 2.6%, less than 2.7%, less than 2.8%, less than 2.9%, or less than 3.0%. The concentration of Cetyl Alcohol in a transdermal delivery formulation may be at least 2.0%, at least 2.1%, at least 2.2%, at least 2.3%, at least 2.4%, at least 2.5%, at least 2.6%, at least 2.7%, at least 2.8%, at least 2.9%, or at least 3.0%. The concentration of Cetyl Alcohol in a transdermal delivery formulation may from about 2.0% to about 2.25%, about 2.25% to about 2.5%, about 2.5% to about 2.75%, about 2.75.5% to about 3%, and any range therebetween.

The concentration of Dextrose in a transdermal delivery formulation may be from about 0.2% to about 2%.

The concentration of Dextrose in a transdermal delivery formulation may be about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, or any amount therebetween. The concentration of Dextrose in a transdermal delivery formulation may be at least 0.2%, at least 0.3%, at least 0.4%, at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, or at least 2.0%. The concentration of Dextrose in a transdermal delivery formulation may be less than 0.2%, less than 0.3%, less than 0.4%, less than 0.5%, less than 0.6%, less than 0.7%, less than 0.8%, less than 0.9%, less than 1.0%, less than 1.1%, less than 1.2%, less than 1.3%, less than 1.4%, less than 1.5%, less than 1.6%, less than 1.7%, less than 1.8%, less than 1.9%, or less than 2.0%. The concentration of Dextrose in a transdermal delivery formulation may be from 0.2% to 0.4%, from 0.4% to 0.6%, from 0.6% to 0.8%, from 0.8% to 1.0%, from 1.0% to 1.2%, from 1.2% to 1.4%, from 1.4% to 1.8%, from 1.6 to 1.68% or from 1.8% to 2.0% and any range therebetween.

The concentration of Ethanol in a transdermal delivery formulation may be from about 1% to about 2%. The concentration of Ethanol in a transdermal delivery formulation may be about 1.0%, about 1.1%, about 1.2%, about 1.3%, about 1.4%, about 1.5%, about 1.6%, about 1.7%, about 1.8%, about 1.9%, about 2.0%, or any amount therebetween. The concentration of Ethanol in a transdermal delivery formulation may be at least 1.0%, at least 1.1%, at least 1.2%, at least 1.3%, at least 1.4%, at least 1.5%, at least 1.6%, at least 1.7%, at least 1.8%, at least 1.9%, or at least 2.0%. The concentration of Ethanol in a transdermal delivery formulation may be less than 1.1%, less than 1.2%, less than 1.3%, less than 1.4%, less than 1.5%, less than 1.6%, less than 1.7%, less than 1.8%, less than 1.9%, or less than 2.0%. The concentration of Dextrose in a transdermal delivery formulation may be from 1.0% to 1.2%, from 1.2% to 1.4%, from 1.4% to 1.8%, from 1.6 to 1.68% or from 1.8% to 2.0% and any range therebetween.

The concentration of lecithin in a transdermal delivery formulation may be from about 5% to about 10%. The concentration of lecithin in a transdermal delivery formulation may be at least 3%, at least 4%, at least 5%, at least 6%, at least 7%, at least 8%, at least 9%, at least 10%, at least 11%, at least 12%, at least 13%, at least 14%, at least 15% or more. The concentration of lecithin in a transdermal delivery formulation may be not more than 3%, not more than 4%, not more than 5%, not more than 6%, not more than 7%, not more than 8%, not more than 9%, not more than 10%, not more than 11%, not more than 12%, not more than 13%, not more than 14%, not more than 15% or more. The concentration of lecithin in a transdermal delivery formulation may be about 3%, about 4%, about 5%, about 6%, about 7%, about 8%, about 9%, about 10%, about 11%, about 12%, about 13%, about 14%, about 15%. The concentration of lecithin in a transdermal delivery formulation may be from 3% to 5%, from 4% to 6%, from 5% to 7%, from 6% to 8%, from 7% to 9%, from 8% to 10%, from 9% to 11%, from 10% to 12%, from 11% to 13%, from 12% to 14%, from 13% to 15%, and any range therebetween.

The concentration of linoleic acid in a transdermal delivery formulation may be from about 1% to about 3%. The concentration of linoleic acid in a transdermal delivery formulation may be at least 1%, at least 2%, at least 2.5%, at least 3%, at least 4%, at least 5% or more. The concentration of linoleic acid in a transdermal delivery formulation may be about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, or any amount therebetween. The concentration of linoleic acid in a transdermal delivery formulation may be from 1% to 5%; from 1.25% to 4%, from 1.5% to 3%, from 1.7% to 2.5% or from 1.6% to 2%. The concentration of linoleic acid in a transdermal delivery formulation may be no more than 1%, no more than 2%, no more than 2.5%, no more than 3%, no more than 4%, no more than 5%.

The concentration of Propylene Glycol in a transdermal delivery formulation may be from about from 3% to about 7%. The concentration of Propylene Glycol in a transdermal delivery formulation may be about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0% or any amount therebetween. The concentration of Propylene Glycol in a transdermal delivery formulation may be less than 3.1%, less than 3.2%, less than 3.3%, less than 3.4%, less than 3.5%, less than 3.6%, less than 3.7%, less than 3.8%, less than 3.9%, less than 4.0%, less than 4.1%, less than 4.2%, less than 4.3%, less than 4.4%, less than 4.5%, less than 4.6%, less than 4.7%, less than 4.8%, less than 4.9%, less than 5.0%, less than 5.1%, less than 5.2%, less than 5.3%, less than 5.4%, less than 5.5%, less than 5.6%, less than 5.7%, less than 5.8%, less than 5.9%, less than 6.0%, less than 6.1%, less than 6.2%, less than 6.3%, less than 6.4%, less than 6.5%, less than 6.6%, less than 6.7%, less than 6.8%, less than 6.9%, less than 7.0%. The concentration of Propylene Glycol in a transdermal delivery formulation may be at least 3.0%, at least 3.1%, at least 3.2%, at least 3.3%, at least 3.4%, at least 3.5%, at least 3.6%, at least 3.7%, at least 3.8%, at least 3.9%, at least 4.0%, at least 4.1%, at least 4.2%, at least 4.3%, at least 4.4%, at least 4.5%, at least 4.6%, at least 4.7%, at least 4.8%, at least 4.9%, at least 5.0%, at least 5.1%, at least 5.2%, at least 5.3%, at least 5.4%, at least 5.5%, at least 5.6%, at least 5.7%, at least 5.8%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%.

The concentration of Stearic Alcohol in a transdermal delivery formulation may be from about 0.4% to about 0.8%.

The concentration of Stearic Alcohol in a transdermal delivery formulation may be about 0.40%, about 0.41%, about 0.42%, about 0.43%, about 0.44%, about 0.45%, about 0.46%, about 0.47%, about 0.48%, about 0.49%, about 0.50%, about 0.51%, about 0.52%, about 0.53%, about 0.54%, about 0.55%, about 0.56%, about 0.57%, about 0.58%, about 0.59%, about 0.60%, about 0.61%, about 0.62%, about 0.63%, about 0.64%, about 0.65%, about 0.66%, about 0.67%, about 0.68%, about 0.69%, about 0.70%, about 0.71%, about 0.72%, about 0.73%, about 0.74%, about 0.75%, about 0.76%, about 0.77%, about 0.78%, about 0.79%, about 0.80%, or any amount therebetween. The concentration of Stearic Alcohol in a transdermal delivery formulation may be less than 0.41%, less than 0.42%, less than 0.43%, less than 0.44%, less than 0.45%, less than 0.46%, less than 0.47%, less than 0.48%, less than 0.49%, less than 0.50%, less than 0.51%, less than 0.52%, less than 0.53%, less than 0.54%, less than 0.55%, less than 0.56%, less than 0.57%, less than 0.58%, less than 0.59%, less than 0.60%, less than 0.61%, less than 0.62%, less than 0.63%, less than 0.64%, less than 0.65%, less than 0.66%, less than 0.67%, less than 0.68%, less than 0.69%, less than 0.70%, less than 0.71%, less than 0.72%, less than 0.73%, less than 0.74%, less than 0.75%, less than 0.76%, less than 0.77%, less than 0.78%, less than 0.79%, or less than 0.80%. The concentration of Stearic Alcohol in a transdermal delivery formulation may be at least 0.40%, at least 0.41%, at least 0.42%, at least 0.43%, at least 0.44%, at least 0.45%, at least 0.46%, at least 0.47%, at least 0.48%, at least 0.49%, at least 0.50%, at least 0.51%, at least 0.52%, at least 0.53%, at least 0.54%, at least 0.55%, at least 0.56%, at least 0.57%, at least 0.58%, at least 0.59%, at least 0.60%, at least 0.61%, at least 0.62%, at least 0.63%, at least 0.64%, at least 0.65%, at least 0.66%, at least 0.67%, at least 0.68%, at least 0.69%, at least 0.70%, at least 0.71%, at least 0.72%, at least 0.73%, at least 0.74%, at least 0.75%, at least 0.76%, at least 0.77%, at least 0.78%, at least 0.79%, or at least 0.80%.

In various embodiments, the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

An additional component in a transdermal delivery formulation of the disclosure is an alcohol. Benzyl alcohol and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 3% w/w, 4% w/w, 5% w/w, 6% w/w, 7% w/w, 8% w/w, 9% w/w, or 10% w/w, and other intermediate weight percentages are included. Due to the aromatic group present in a transdermal delivery formulation such as benzyl alcohol, the molecule has a polar end (the alcohol end) and a non-polar end (the benzene end). This enables the agent to dissolve a wider variety of transdermal delivery formulation components.

Suitable gelling components also include isopropyl palmitate, ethyl laurate, ethyl myristate and isopropyl myristate. In some embodiments, a transdermal delivery formulation comprises a gelling agent in an amount less than 5% w/w of a transdermal delivery formulation. Certain hydrocarbons, such as cyclopentane, cyclooctane, trans-decalin, trans-pinane, n-pentane, n-hexane, n-hexadecane may also be used. In some embodiments, the transdermal delivery formulation comprises a mixture of xanthan gum, sclerotium gum, pullulan, or a combination thereof in an amount less than 2% w/w, 5% w/w, or 10% w/w of the formulation. In some embodiments, a transdermal delivery formulation comprises Siligel™ in an amount from about 1 to about 5% w/w or from about 5 to about 15% w/w, or an equivalent mixture of xanthan gum, sclerotium gum, and pullulan. In some embodiments, a transdermal delivery formulation comprises a mixture of caprylic triglycerides and capric triglycerides in amount less than 2% w/w, 8% w/w, or 10% w/w of the formulation. In some embodiments, a transdermal delivery formulation comprises Myritol® 312 in an amount from about 0.5 to about 10% w/w, or an equivalent mixture of caprylic triglycerides and capric triglycerides.

In some embodiments, as noted above, the performance of a transdermal delivery formulation is further improved by including a nonionic detergent and polar gelling agent or including a powdered surfactant. In both aqueous and anhydrous forms of the composition, detergents, typically nonionic detergents are added.

In general, the nonionic detergent should be present in an amount from about 1% w/w to about 30% w/w of a transdermal delivery formulation. Typically, in the compositions wherein a transdermal delivery formulation is topped off with a polar or aqueous solution containing detergent, the amount of detergent is relatively low—e.g., 2-25% w/w, or 5-15% w/w or 7-12% w/w of a transdermal delivery formulation.

However, in compositions that are essentially anhydrous and are topped-off by powdered detergent, relatively higher percentages are usually used—e.g., 20-60% w/w.

In some embodiments, a transdermal delivery formulation further comprises a detergent portion in an amount from about 1 to about 70% w/w or from about 1 to about 60% w/w of a transdermal delivery formulation. In some embodiments, the nonionic detergent provides suitable handling properties whereby the formulations are gel-like or creams at room temperature. To exert this effect, the detergent, typically a poloxamer, is present in an amount from about 2 to about 12% w/w of a transdermal delivery formulation, preferably from about 5 to about 25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a transdermal delivery formulation also contains surfactant, typically, nonionic surfactant at 2-25% w/w of a transdermal delivery formulation along with a polar solvent wherein the polar solvent is present in an amount at least in molar excess of the nonionic surfactant. In these embodiments, typically, the composition comprises the above-referenced amounts of a transdermal delivery formulation and benzyl alcohol along with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount from about 1 to about 30% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/w sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

Additional components that can also be included in a transdermal delivery formulation are fatty acids, terpenes, lipids, and cationic, and anionic detergents. In some embodiments, a transdermal delivery formulation further comprises tranexamic acid in an amount less than 2% w/w, 5% w/w, or 10% w/w of the formulation. In some embodiments, a transdermal delivery formulation further comprises a polar solvent in an amount less than 2% w/w, 5% w/w, 10% w/w, or 20% w/w of the transdermal delivery formulation. In some embodiments, a transdermal delivery formulation further comprises a humectant, an emulsifier, an emollient, or a combination thereof. In some embodiments, a transdermal delivery formulation further comprises almond oil in an amount less than about 5% w/w. In some embodiments, a formulation further comprises a mixture of thermoplastic polyurethane and polycarbonate in an amount less than about 5% w/w. In some embodiments, a transdermal delivery formulation further comprises phosphatidylethanolamine in an amount less than about 5% w/w. In some embodiments, a transdermal delivery formulation further comprises an inositol phosphatide in an amount less than about 5% w/w.

Other solvents and related compounds that can be used in some embodiments include acetamide and derivatives, acetone, n-alkanes (chain length from 7 to 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C3-C6 triols or their mixtures and a polar lipid compound selected from C16 or C18 monounsaturated alcohol, C16 or C18 branched saturated alcohol and their mixtures, propylene glycol, sorbitan monolaurate sold as Span@ 20 by Sigma-Aldrich, squalene, triacetin, trichloroethanol, trifluoroethanol, trimethylene glycol and xylene.

Fatty alcohols, fatty acids, fatty esters, are bilayer fluidizers that can be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleate, sorbitan trilaurate, sorbitan trioleate, sucrose coconut fatty ester mixtures, sucrose monolaurate, sucrose monooleate, tetradecyl N.N-dimethylamino acetate.

Examples of suitable fatty acid—include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that can be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants can be included, such as vitamin C, vitamin E, proanthocyanidin and a-lipoic acid typically in concentrations of 0.1%-2.5% w/w.

In some applications, it is desirable to adjust the pH of a transdermal delivery formulation to assist in permeation or to adjust the nature of the target compounds in the subject. In some instances, the pH is adjusted to a level of pH 9-11 or 10-11 which can be done by providing appropriate buffers or simply adjusting the pH with base.

In some applications a formulation for transdermal delivery may, for example, comprise: Aveeno®, for example in an amount from about 10 to about 95% w/w; from about 20 to about 85% w/w, from about 20 to about −75% w/w, from about 20 to about 50% w/w.

As disclosed herein, a transdermal formulation may comprise a medicament, agent, or another ingredient (e.g., menthol, colchicine, nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase), the amount of water may be reduced accordingly. Thus, the amounts of water in the illustrative formulations of Tables 2 to Table 19 will have a reduced amount of water. As an example, colchicine may be included in a transdermal formulation in an amount from 0.02% to about 0.4% w/w, e.g., about 0.03% to about 0.36% w/w.

The medicament, agent, or another ingredient may be present in a transdermal formulation in a percentage from about 0.001% to about 0.1% (w/w), e.g., about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1%, or any percentage therebetween.

The medicament, agent, or another ingredient may be present in a transdermal formulation in an amount from about 0.1% to about 1.0% (w/w), e.g., about 0.1%, 0.2%, 0.3%, 0.4%, 0.5%, 0.6%, 0.7%, 0.8%, 0.9%, or 1.0% or any percentage therebetween. As examples, about 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, or 1.00%, or any percentage therebetween. The ranges may be from about 0.1% to about 0.2%, from about 0.1% to about 0.3%, from about 0.1% to about 0.5%, from about 0.1% to about 0.7%, from about 0.1% to about 0.8%, from about 0.2% to about 0.3%, from about 0.2% to about 0.4%, from about 0.2% to about 0.6%, from about 0.2% to about 0.8%, from about 0.2% to about 0.9%, from about 0.2% to about 0.5%, from about 0.2% to about 0.7%, from about 0.3% to about 0.4%, from about 0.3% to about 0.5%, from about 0.3% to about 0.7%, from about 0.3% to about 0.9%, from about 0.3% to about 1.0% from about 0.3% to about 0.6%, from about 0.3% to about 0.8%, from about 0.4% to about 0.5%, from about 0.4% to about 0.6%, from about 0.4% to about 0.8%, from about 0.4% to about 1.0%, from about 0.4% to about 0.7%, from about 0.4% to about 0.9%, from about 0.5% to about 0.6%, from about 0.5% to about 0.7%, from about 0.5% to about 0.9%, from about 0.5% to about 0.8%, from about 0.5% to about 1.0%, from about 0.6% to about 0.7%, from about 0.6% to about 0.8%, from about 0.6% to about 1.0%%, from about 0.6% to about 0.9%, from about 0.7% to about 0.8%, from about 0.7% to about 0.9%, from about 0.7% to about 1.0%, from about 0.8% to about 0.9%, from about 0.8% to about 1.0%, or from about 0.9% to about 1.0%, and any subrange therebetween.

The medicament, agent, or another ingredient may be present in a transdermal formulation in an amount from about 1% to about 10% (w/w), e.g., about 1%, 2%, 3%, 4%, 5%, 6%, 7%, 8%, 9%, or 10% or any percentage therebetween. As examples, about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, or any percentage therebetween. The ranges may be from about 1% to about 2%, from about 1% to about 3%, from about 1% to about 5%, from about 1% to about 7%, from about 1% to about 8%, from about 2% to about 3%, from about 2% to about 4%, from about 2% to about 6%, from about 2% to about 8%, from about 2% to about 9%, from about 2% to about 5%, from about 2% to about 7%, from about 3% to about 4%, from about 3% to about 5%, from about 3% to about 7%, from about 3% to about 9%, from about 3% to about 10% from about 3% to about 6%, from about 3% to about 8%, from about 4% to about 5%, from about 4% to about 6%, from about 4% to about 8%, from about 4% to about 10%, from about 4% to about 7%, from about 4% to about 9%, from about 5% to about 6%, from about 5% to about 7%, from about 5% to about 9%, from about 5% to about 8%, from about 5% to about 10%, from about 6% to about 7%, from about 6% to about 8%, from about 6% to about 10%%, from about 6% to about 9%, from about 7% to about 8%, from about 7% to about 9%, from about 7% to about 10%, from about 8% to about 9%, from about 8% to about 10%, or from about 9% to about 10%, and any subrange therebetween.

The medicament, agent, or another ingredient may be present in a transdermal formulation in an amount from about 11% to about 20% (w/w), e.g., about 11%, 12%, 13%, 14%, 15%, 16%, 17%, 18%, 19%, or 20% or any percentage therebetween. As examples, about 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, or any percentage therebetween. The ranges may be from about 11% to about 12%, from about 11% to about 13%, from about 11% to about 15%, from about 11% to about 17%, from about 11% to about 18%, from about 12% to about 13%, from about 12% to about 14%, from about 12% to about 16%, from about 12% to about 18%, from about 12% to about 19%, from about 12% to about 15%, from about 12% to about 17%, from about 13% to about 14%, from about 13% to about 15%, from about 13% to about 17%, from about 13% to about 19%, from about 13% to about 20% from about 13% to about 16%, from about 13% to about 18%, from about 14% to about 15%, from about 14% to about 16%, from about 14% to about 18%, from about 14% to about 20%, from about 14% to about 17%, from about 14% to about 19%, from about 15% to about 16%, from about 15% to about 17%, from about 15% to about 19%, from about 15% to about 18%, from about 15% to about 20%, from about 16% to about 17%, from about 16% to about 18%, from about 16% to about 20%%, from about 16% to about 19%, from about 17% to about 18%, from about 17% to about 19%, from about 17% to about 20%, from about 18% to about 19%, from about 18% to about 20%, or from about 19% to about 20%, and any subrange therebetween.

The medicament, agent, or another ingredient may be present in a transdermal formulation in an amount from about 21% to about 30% (w/w), e.g., about 21%, 22%, 23%, 24%, 25%, 26%, 27%, 28%, 29%, or 30% or any percentage therebetween. As examples, about 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.0%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 29.0%, 29.1%, 29.2%, 29.3%, 29.4%, 29.5%, 29.6%, 29.7%, 29.8%, 29.9%, 30.0%, or any percentage therebetween. The ranges may be from about 21% to about 22%, from about 21% to about 23%, from about 21% to about 25%, from about 21% to about 27%, from about 21% to about 28%, from about 22% to about 23%, from about 22% to about 24%, from about 22% to about 26%, from about 22% to about 28%, from about 22% to about 29%, from about 22% to about 25%, from about 22% to about 27%, from about 23% to about 24%, from about 23% to about 25%, from about 23% to about 27%, from about 23% to about 29%, from about 23% to about 30% from about 23% to about 26%, from about 23% to about 28%, from about 24% to about 25%, from about 24% to about 26%, from about 24% to about 28%, from about 24% to about 30%, from about 24% to about 27%, from about 24% to about 29%, from about 25% to about 26%, from about 25% to about 27%, from about 25% to about 29%, from about 25% to about 28%, from about 25% to about 30%, from about 26% to about 27%, from about 26% to about 28%, from about 26% to about 30%%, from about 26% to about 29%, from about 27% to about 28%, from about 27% to about 29%, from about 27% to about 30%, from about 28% to about 29%, from about 28% to about 30%, or from about 29% to about 30%, and any subrange therebetween.

The medicament, agent, or another ingredient may be present in a transdermal formulation in an amount from about 31% to about 40% (w/w), e.g., about 31%, 32%, 33%, 34%, 35%, 36%, 37%, 38%, 39%, or 40% or any percentage therebetween. As examples, about 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, or any percentage therebetween. The ranges may be from about 31% to about 32%, from about 31% to about 33%, from about 31% to about 35%, from about 31% to about 37%, from about 31% to about 38%, from about 32% to about 33%, from about 32% to about 34%, from about 32% to about 36%, from about 32% to about 38%, from about 32% to about 39%, from about 32% to about 35%, from about 32% to about 37%, from about 33% to about 34%, from about 33% to about 35%, from about 33% to about 37%, from about 33% to about 39%, from about 33% to about 40%, from about 33% to about 36%, from about 33% to about 38%, from about 34% to about 35%, from about 34% to about 36%, from about 34% to about 38%, from about 34% to about 40%, from about 34% to about 37%, from about 34% to about 39%, from about 35% to about 36%, from about 35% to about 37%, from about 35% to about 39%, from about 35% to about 38%, from about 35% to about 40%, from about 36% to about 37%, from about 36% to about 38%, from about 36% to about 40%%, from about 36% to about 39%, from about 37% to about 38%, from about 37% to about 39%, from about 37% to about 40%, from about 38% to about 39%, from about 38% to about 40%, or from about 39% to about 40%, and any subrange therebetween.

The medicament, agent, or another ingredient may be present in a transdermal formulation in an amount from about 41% to about 50% (w/w), e.g., about 41%, 42%, 43%, 44%, 45%, 46%, 47%, 48%, 49%, or 40% or any percentage therebetween. As examples, about 41.0%, 41.1%, 41.2%, 41.3%, 41.4%, 41.5%, 41.6%, 41.7%, 41.8%, 41.9%, 42.0%, 42.1%, 42.2%, 42.3%, 42.4%, 42.5%, 42.6%, 42.7%, 42.8%, 42.9%, 43.0%, 43.1%, 43.2%, 43.3%, 43.4%, 43.5%, 43.6%, 43.7%, 43.8%, 43.9%, 44.0%, 44.1%, 44.2%, 44.3%, 44.4%, 44.5%, 44.6%, 44.7%, 44.8%, 44.9%, 45.0%, 45.1%, 45.2%, 45.3%, 45.4%, 45.5%, 45.6%, 45.7%, 45.8%, 45.9%, 46.0%, 46.1%, 46.2%, 46.3%, 46.4%, 46.5%, 46.6%, 46.7%, 46.8%, 46.9%, 47.0%, 47.1%, 47.2%, 47.3%, 47.4%, 47.5%, 47.6%, 47.7%, 47.8%, 47.9%, 48.0%, 48.1%, 48.2%, 48.3%, 48.4%, 48.5%, 48.6%, 48.7%, 48.8%, 48.9%, 49.0%, 49.1%, 49.2%, 49.3%, 49.4%, 49.5%, 49.6%, 49.7%, 49.8%, 49.9%, 50.0%, or any percentage therebetween. The ranges may be from about 41% to about 42%, from about 41% to about 43%, from about 41% to about 45%, from about 41% to about 47%, from about 41% to about 48%, from about 42% to about 43%, from about 42% to about 44%, from about 42% to about 46%, from about 42% to about 48%, from about 42% to about 49%, from about 42% to about 45%, from about 42% to about 47%, from about 43% to about 44%, from about 43% to about 45%, from about 43% to about 47%, from about 43% to about 49%, from about 43% to about 50% from about 43% to about 46%, from about 43% to about 48%, from about 44% to about 45%, from about 44% to about 46%, from about 44% to about 48%, from about 44% to about 50%, from about 44% to about 47%, from about 44% to about 49%, from about 45% to about 46%, from about 45% to about 47%, from about 45% to about 49%, from about 45% to about 48%, from about 45% to about 50%, from about 46% to about 47%, from about 46% to about 48%, from about 46% to about 50%%, from about 46% to about 49%, from about 47% to about 48%, from about 47% to about 49%, from about 47% to about 50%, from about 48% to about 49%, from about 48% to about 50%, or from about 49% to about 50%, and any subrange therebetween.

Other formulations and methods relevant to the present disclosure are disclosed in PCT/US2018/028017 and PCT/US2022/012330, the entire contents of each of which is incorporated by reference in its entirety.

Pluralities of formulations An aspect of the present disclosure is a plurality of formulations comprising any herein disclosed transdermal formulation and a second composition comprising a nonsteroidal medicament.

In embodiments, the nonsteroidal medicament is a nonsteroidal anti-inflammatory drug (NSAID), a COX-2 Inhibitor, an Opioid, and/or Illaris (canakinumab).

In some embodiments, the second composition comprises colchicine.

In embodiments, the second composition is administered orally or topically before, contemporaneously with, and/or after administering the transdermal formulation.

In various embodiments, the transdermal formulation is as described in any of Table 1 to Table 19 or as described elsewhere herein.

Another aspect of the present disclosure is a plurality of formulations comprising any herein disclosed transdermal formulation and a second composition comprising a corticosteroid.

In embodiments, the corticosteroid is one or more of prednisone, methylprednisolone, prednisolone, triamcinolone, and/or a glucocorticoid.

In some embodiments, the second composition comprises colchicine.

In various embodiments, the second composition is administered orally or topically before, contemporaneously with, and/or after administering the transdermal formulation.

In some embodiments, the transdermal formulation is as described in any of Table 1 to Table 19 or as described elsewhere herein.

A further aspect of the present disclosure is a plurality of formulations comprising any herein disclosed transdermal formulation and a second composition comprising a nonsteroidal medicament.

In various embodiments, the nonsteroidal medicament is a nonsteroidal anti-inflammatory drug (NSAID), a COX-2 Inhibitor, an Opioid, and/or Illaris (canakinumab).

In embodiments, the second composition is administered orally or topically before, contemporaneously with, and/or after administering the transdermal formulation.

In some embodiments, the transdermal formulation is as described in any of Table 1 to Table 19 or as described elsewhere herein.

An additional aspect of the present disclosure is a plurality of formulations comprising any herein disclosed transdermal formulation and a second composition comprising a corticosteroid.

In embodiments, the corticosteroid is one or more of prednisone, methylprednisolone, prednisolone, triamcinolone, and/or a glucocorticoid.

In some embodiments, the second composition is administered orally or topically before, contemporaneously with, and/or after administering the transdermal formulation.

In various embodiments, the transdermal formulation is as described in any of Table 1 to Table 19 or as described elsewhere herein.

### Methods

An aspect of the present disclosure is a method for treating or reducing inflammation in a subject in need thereof: The method comprising administering to the subject a combination therapy comprising a transdermal formulation comprising a therapeutically-effective amount of a buffering agent and a separate composition comprising a therapeutically-effective amount of colchicine, wherein the colchicine is administered before, contemporaneously with, and/or after administering the transdermal formulation. In this method, the combination therapy reduces blood or serum levels of C-Reactive Protein relative to a subject that is not administered the combination therapy.

Another aspect of the present disclosure is a method for treating or reducing inflammation in a subject in need thereof: The method comprising administering to the subject a transdermal formulation comprising a therapeutically-effective amount of a buffering agent and a therapeutically-effective amount of colchicine.

In this method, the transdermal formulation reduces blood or serum levels of C-Reactive Protein relative to a subject that is not administered the transdermal formulation.

A further aspect of the present disclosure is a method for preventing inflammation and/or reducing the severity of upcoming inflammation: The method comprising administering to a subject who is not experiencing a symptom of inflammation, a combination therapy comprising a transdermal formulation comprising a therapeutically-effective amount of a buffering agent and a separate composition comprising a therapeutically-effective amount of colchicine, wherein the colchicine is administered before, contemporaneously with, and/or after administering the transdermal formulation. In this method, preventing inflammation and/or reducing the severity of upcoming inflammation is determined in part by a reduction of blood or serum levels of C-Reactive Protein relative to a subject that is not administered the combination therapy.

An additional aspect of the present disclosure is a method for preventing inflammation and/or reducing the severity of upcoming inflammation: The method comprising administering to a subject who is not experiencing a symptom of inflammation, a transdermal formulation comprising a therapeutically-effective amount of a buffering agent and a therapeutically-effective amount of colchicine. In this method, the transdermal formulation is administered before symptoms inflammation are experienced by the subject and preventing inflammation and/or reducing the severity of upcoming inflammation is determined in part by a reduction of blood or serum levels of C-Reactive Protein relative to a subject that is not administered the transdermal formulation.

In an aspect, the present disclosure provides a method for reducing the likelihood a recurrent inflammation, the method comprising administering to a subject that previously has been treated for inflammation, a combination therapy comprising a transdermal formulation comprising a therapeutically-effective amount of a buffering agent and a separate composition comprising a therapeutically-effective amount of colchicine, wherein the colchicine is administered before, contemporaneously with, and/or after administering the transdermal formulation. In this method, the combination therapy reduces blood or serum levels of C-Reactive Protein relative to a subject that is not administered the combination therapy.

In another aspect, the present disclosure provides a method for reducing the likelihood a recurrent inflammation, the method comprising administering to a subject that previously has been treated for inflammation, a transdermal formulation comprising a therapeutically-effective amount of a buffering agent and a therapeutically-effective amount of colchicine. In this method, the combination therapy reduces blood or serum levels of C-Reactive Protein relative to a subject that is not administered the transdermal formulation.

In a further aspect, the present disclosure provides a method for treating severe pain associated with inflammation, the method comprising administering to a subject having severe pain associated with inflammation a combination therapy comprising administering to the subject a transdermal formulation comprising a therapeutically-effective amount of a buffering agent; administering to the subject a separate composition comprising a therapeutically-effective amount of colchicine; and administering to the subject one of (a) a composition comprising a nonsteroidal medicament or (b) a composition comprising a corticosteroid. In this method, severe pain associated with the inflammation being defined as an ACR score of 7 to 10 and the combination therapy reduces blood or serum levels of C-Reactive Protein relative to a subject that is not administered the combination therapy.

In an additional aspect, the present disclosure provides a method for treating severe pain associated with inflammation, the method comprising administering to a subject having severe pain associated with inflammation a combination therapy comprising administering to the subject a transdermal formulation comprising: a therapeutically-effective amount of a buffering agent, and a therapeutically-effective amount of colchicine and administering to the subject one or both of (a) a composition comprising a nonsteroidal medicament or (b) a composition comprising a corticosteroid. In this method, severe pain associated with the inflammation being defined as an ACR score of 7 to 10 and the combination therapy reduces blood or serum levels of C-Reactive Protein relative to a subject that is not administered the combination therapy.

An aspect of the present disclosure is a method for treating mild to moderate pain associated with inflammation, the method comprising administering to a subject having mild to moderate pain associated with inflammation a combination therapy comprising administering to the subject a transdermal formulation comprising a therapeutically-effective amount of a buffering agent and administering to the subject one of (a) a composition comprising colchicine, (b) a composition comprising a nonsteroidal medicament, or (c) a composition comprising a corticosteroid. In this method, mild pain associated with the inflammation being defined as an ACR score of up to 4 and moderate pain associated with the inflammation being defined as an ACR score of 5 or 6 and the combination therapy reduces blood or serum levels of C-Reactive Protein relative to a subject that is not administered the combination therapy.

Another aspect of the present disclosure is a method for treating a bone density disorder in a subject in need thereof. The method comprising administering to the subject a combination therapy comprising: a transdermal formulation comprising a therapeutically-effective amount of a buffering agent; and a separate composition comprising a therapeutically-effective amount of colchicine, wherein the colchicine is administered before, contemporaneously with, and/or after administering the transdermal formulation. In this aspect, the combination therapy: lowers elevated calcium levels in blood or plasma, stabilizes calcium levels in blood or plasma to levels prior to a gout flare, reduces symptoms related to osteoporosis, reduces symptoms related to osteomalacia, improves bone density, and/or inhibits and/or reverses bone decalcification.

A further aspect of the present disclosure is a method for treating or reducing inflammation in a subject in need thereof: The method comprising administering to the subject a transdermal formulation comprising a therapeutically-effective amount of a buffering agent. In this method, the transdermal formulation reduces blood or serum levels of C-Reactive Protein relative to a subject that is not administered the combination therapy.

An additional aspect of the present disclosure is a method for treating or reducing inflammation in a subject in need thereof: The method comprises administering to the subject a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy.

In an aspect, the present disclosure provides a method for treating or reducing inflammation in a subject in need thereof, the method comprising administering to the subject a transdermal formulation comprises a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

In another aspect, the present disclosure provides a method for preventing inflammation and/or reducing the severity of upcoming inflammation, the method comprising administering to a subject who is not experiencing a symptom of inflammation a transdermal formulation comprising a therapeutically-effective amount of a buffering agent. In this method, the transdermal formulation reduces blood or serum levels of C-Reactive Protein relative to a subject that is not administered the combination therapy.

In a further aspect, the present disclosure provides a method for preventing inflammation and/or reducing the severity of upcoming inflammation, the method comprising administering to a subject who is not experiencing a symptom of inflammation, a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy

In an additional aspect, the present disclosure provides a method for preventing inflammation and/or reducing the severity of upcoming inflammation, the method comprising administering to a subject who is not experiencing a symptom of inflammation, a transdermal formulation comprising a therapeutically-effective amount of a buffering agent and a therapeutically-effective amount of a nonsteroidal medicament and/or a corticosteroid. In this method, the transdermal formulation is administered before symptoms inflammation are experienced by the subject and prevents inflammation and/or reduces the severity of upcoming inflammation is determined in part by a reduction of blood or serum levels of C-Reactive Protein relative to a subject that is not administered the combination therapy.

An aspect of the present disclosure is a method for reducing the likelihood a recurrent inflammation, the method comprising administering to a subject that previously has been treated for inflammation, a transdermal formulation comprising a therapeutically-effective amount of a buffering agent. In this method, the transdermal formulation reduces blood or serum levels of C-Reactive Protein relative to a subject that is not administered the combination therapy.

Another aspect of the present disclosure is a method for reducing the likelihood a recurrent inflammation, the method comprising administering to a subject that previously has been treated for inflammation, a transdermal formulation comprising a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

A further aspect of the present disclosure is a method for reducing the likelihood a recurrent inflammation, the method comprising administering to a subject that previously has been treated for inflammation, administering to the subject a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the transdermal formulation reduces blood or serum levels of C-Reactive Protein relative to a subject that is not administered the combination therapy.

An additional aspect of the present disclosure is a method for treating chronic inflammation, the method comprising administering to a subject that previously has been treated for inflammation, a transdermal formulation comprising a therapeutically-effective amount of a buffering agent. In this method, the transdermal formulation reduces blood or serum levels of C-Reactive Protein relative to a subject that is not administered the combination therapy.

In an aspect, the present disclosure provides a method for treating chronic inflammation, the method comprising administering to a subject that previously has been treated for inflammation, a transdermal formulation comprising a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

In another aspect, the present disclosure provides a method for treating chronic inflammation, the method comprising administering to a subject that previously has been treated for inflammation, a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy.

In a further aspect, the present disclosure provides a method for treating a bone density disorder in a subject in need thereof, the method comprising administering to the subject a transdermal formulation comprising a therapeutically-effective amount of a buffering agent. In this method, the transdermal formulation lowers elevated calcium levels in blood or plasma, stabilizes calcium levels in blood or plasma to levels prior to a gout flare, reduces symptoms related to osteoporosis, reduces symptoms related to osteomalacia, improves bone density, and/or inhibits and/or reverses bone decalcification.

In each of these aspects, any herein-disclosed formulation may be administered to a subject in need.

In embodiments, the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

As an example, a method may comprise administering a transdermal formulation as disclosed in Table 1 to Table 19. The transdermal formulation may comprise nine or more, ten or more, eleven or more, twelve or more, thirteen or more, fourteen or more, fifteen or more, sixteen or more, seventeen or more, or eighteen of the following ingredients: Almond Oil, Benzyl Alcohol, Buffering Agent, Cetyl Alcohol, Deionized Water, Dextrose, Ethanol, Isopropyl Palmitate, Lecithin, Linoleic Acid, Menthol, Oleic Acid, Phosphatidylcholine, Poloxamer 407, Polyglyceryl-4 Laurate, Propylene Glycol, Safflower Oil, Stearic Acid, and Stearic Alcohol. The buffering agent may be Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

As another example, a method may comprise administering a transdermal formulation as disclosed in Table 1 to Table 19. The transdermal formulation may comprise nine or more, ten or more, eleven or more, twelve or more, thirteen or more, fourteen or more, fifteen or more, sixteen or more, seventeen or more or eighteen of the following ingredients and in the following amounts: Almond Oil from about 2% to about 4%; Benzyl Alcohol from about 0.9% to about 2%; Buffering Agent from about 30% to about 33%; Cetyl Alcohol from about 2% to about 3%; Deionized Water from about 25% to about 75%; Dextrose from about 0.2% to about 2%; Ethanol from about 1% to about 2%; Isopropyl Palmitate from about 8% to about 15%; lecithin from about 5% to about 10%; linoleic Acid from about 1% to about 3%; Menthol from about 0.1% to about 5%; Oleic Acid from about 0.5% to about 2%; Phosphatidylcholine from about 3% to about 9%; Poloxamer 407 from about 5% to about 10%; Polyglyceryl-4 Laurate from about 0.5% to about 2%; Propylene Glycol from about from 3% to about 7%; Safflower Oil from about from 1% to about 3%; Stearic Acid from about 0.2% to about 1%; and Stearic Alcohol from about 0.4% to about 0.8%. The buffering agent may be Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

In a further example, a method may comprise administering an illustrative transdermal formulation. The illustrative transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

In these methods, a transdermal formulation may comprise an additional ingredient (e.g., menthol, colchicine, nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase), the amount of water may be reduced accordingly. Thus, the amounts of water in the illustrative formulations of Tables 2 to Table 19 will have a reduced amount of water. As an example, colchicine may be included in a transdermal formulation in an amount from 0.02% to about 0.4% w/w, e.g., about 0.03% to about 0.36% w/w.

In these methods, the amount of buffering agent in a transdermal formulation may be at least about 5% (w/w) to about 40% (w/w). In some embodiments, the amount of buffering agent in the formulation is at least about 10% (w/w) to about 40% (w/w). In some embodiments, the amount of buffering agent in the formulation is at least about 15% (w/w) to about 40% (w/w). In some embodiments, the amount of buffering agent in the formulation is at least about 20% (w/w) to about 40% (w/w). In some embodiments, the amount of buffering agent in the formulation is at least about 25% (w/w) to about 40% (w/w). In various embodiments, the amount of buffering agent in the formulation is at least about 5% (w/w) to about 35% (w/w). In various embodiments, the amount of buffering agent in the formulation is at least about 10% (w/w) to about 35% (w/w). In various embodiments, the amount of buffering agent in the formulation is at least about 15% (w/w) to about 35% (w/w). In various embodiments, the amount of buffering agent in the formulation is at least about 20% (w/w) to about 35% (w/w). In various embodiments, the amount of buffering agent in the formulation is at least about 25% (w/w) to about 35% (w/w).

A transdermal delivery formulation provided herein can be topically administered in any form. For administration for the treatment of skin conditions a sufficient amount of the topical composition can be applied onto a desired area and surrounding skin, for example, in an amount sufficient to cover a desired skin surface. A transdermal delivery formulation can be applied to any skin surface, including for example, facial skin, and the skin of the hands, neck, chest and/or scalp.

In applying a transdermal delivery formulation of the invention, a transdermal delivery formulation itself is simply placed on the skin and spread across the surface and/or massaged to aid in penetration. The amount of transdermal delivery formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a transdermal delivery formulation to the skin and prevents distortion of a transdermal delivery formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the herein disclosed formulations that penetrate intact skin may be applied using occlusive patches, such as those in the form micro-patches. External reservoirs of the formulations for extended administration may also be employed.

In embodiments, the separate composition is administered orally or topically before, contemporaneously with, and/or after administering the transdermal formulation.

In some embodiments, the transdermal formulation is administered before, contemporaneously with, and/or after (a) the composition comprising the nonsteroidal medicament or (b) the composition comprising the corticosteroid. In some cases, the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In various embodiments, the therapeutically-effective amount of colchicine is administered orally or topically before, contemporaneously with, and/or after administering the transdermal formulation. In some cases, the dosage of the therapeutically-effective amount of the buffering agent is less than the dosage used to previously treat inflammation and the dosage of the therapeutically-effective amount of colchicine is less than the dosage used to previously treat inflammation or the therapeutically-effective amount of colchicine is the same as the dosage used to previously treat inflammation. In various cases, the therapeutically-effective amount of the buffering agent is the same as the dosage used to previously treat inflammation and the dosage of the therapeutically-effective amount of colchicine is less than the dosage used to previously treat inflammation or the therapeutically-effective amount of colchicine is the same as the dosage used to previously treat inflammation. In some cases, the therapeutically-effective amount of colchicine comprises from about 0.2 mg to about 4 mg, e.g., about 0.3 mg to about 3.6 mg and/or be present in an amount from 0.02% to about 0.4% w/w of the formulation, e.g., about 0.03% to about 0.36% w/w.

In embodiments, administering the combination therapy further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to a gout flare, reduces symptoms related to osteoporosis, reduces symptoms related to osteomalacia, improves bone density, inhibits and/or reverses bone decalcification, and/or increases patient satisfaction.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

The present transdermal delivery formulation remains stable in storage for periods including up to about 5 years, from about 3 months to about 5 years, from about 3 months to about 4 years, from about 3 months to about 3 years, and alternately any time period from about 6 months to about 3 years.

A transdermal delivery formulation described herein remains stable for up to at least 3 years at a temperature of less than or equal to 40° C. In an embodiment, the presently described transdermal delivery formulation remains stable for at least 2 years at a temperature of less than or equal to 40° C. In an embodiment, the presently described transdermal delivery formulation remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described transdermal delivery formulation in accordance with the subject matter described herein remains stable for an extended period of time when packaged in a multi-use container such as a bottle dispenser or the like and exhibits equal to or even greater stability when packaged in a single-use package.

Dosing can be single dosage or cumulative (serial dosing), and can be readily determined by one skilled in the art. A transdermal delivery formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a transdermal delivery formulation as disclosed herein.

Alternatively, treatment of a disease may comprise multiple administrations of an effective dose of a transdermal delivery formulation as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a transdermal delivery formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a transdermal delivery formulation disclosed herein that is administered can be adjusted accordingly.

In an embodiment, the period of administration of a transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

As noted previously, some formulations, compositions, and/or methods of the present disclosure do not comprise colchicine and it is well established that colchicine can cause direct toxicity of the kidneys and colchicine use is counter indicated for subjects with kidney impairment, for example in subjects with diabetes, chronic kidney disease (CKD), Polycystic kidney disease (PKD), Lupus nephritis, kidney cancer, Alport syndrome, amyloidosis, Goodpasture syndrome, and Wegener's granulomatosis, and/or recipients of a renal transplant. Since the formulations, compositions, and/or methods of the present disclosure do not comprise colchicine, they may be safely used in subjects with kidney impairment and this population of patients is especially benefited by and treated with the formulations, compositions, and/or methods of the present disclosure.

Other formulations and methods relevant to the present disclosure are disclosed in PCT/US2018/028017 and PCT/US2022/012330, the entire contents of each of which is incorporated by reference in its entirety. Compositions In aspects and embodiments of the present disclosure, a transdermal formulation may be provided along with an anti-inflammatory or related medicament. The anti-inflammatory or related medicament may be colchicine, a nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent Probenecid or Krystexxa (pegloticase). The anti-inflammatory or related medicament will be in a composition suitable for administration to a subject in need, e.g., a pharmaceutical composition.

The amount of an anti-inflammatory or related medicament may according to the standard dosage for the particular anti-inflammatory or related medicament and according to the standard administration method. That is, if a specific medicament is normally prescribed per os at a specific dosage, then in methods and compositions of the present disclosure, the specific medicament may be administered according to the herein disclosed methods at the dosage normally when prescribed per os. However, in some methods and compositions of the present disclosure, the specific medicament may be administered according to the herein disclosed methods at the dosage lower than when prescribed per os. In some cases, the specific medicament is normally prescribed per os, but in methods of the present disclosure, the specific medicament is included in a transdermal formulation, the dosage of the specific medicament according to the herein disclosed methods may be the same dosage as when prescribed per os or may be less than the dosage when prescribed per os.

For example, the anti-inflammatory or related medicament may be provided in an amount from about 0.0001 mg to about 1 mg, e.g., about 0.0001 mg, 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.015 mg, 0.02 mg, 0.025 mg, 0.03 mg, 0.035 mg, 0.04 mg, 0.045 mg, 0.05 mg, 0.055 mg, 0.06 mg, 0.065 mg, 0.07 mg, 0.075 mg, 0.08 mg, 0.085 mg, 0.09 mg, 0.095 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, or any amount therebetween.

A medicament, the anti-inflammatory or related medicament may be provided at range of amounts from about 1 mg to about 10 mg. As used herein a range from about 1 mg to about 10 mg includes all amounts therebetween and any subranges therebetween. More specifically, the amounts may be about 1 mg, about 1.01 mg, about 1.02 mg, about 1.03 mg, about 1.04 mg, about 1.05 mg, about 1.06 mg, about 1.07 mg, about 1.08 mg, about 1.09 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, and any dosage therebetween. Moreover, the ranges may be from about 1 mg to about 2 mg, from about 1 mg to about 3 mg, from about 1 mg to about 5 mg, from about 1 mg to about 7 mg, from about 1 mg to about 8 mg, from about 2 mg to about 3 mg, from about 2 mg to about 4 mg, from about 2 mg to about 6 mg, from about 2 mg to about 8 mg, from about 2 mg to about 9 mg, from about 2 mg to about 5 mg, from about 2 mg to about 7 mg, from about 3 mg to about 4 mg, from about 3 mg to about 5 mg, from about 3 mg to about 7 mg, from about 3 mg to about 9 mg, from about 3 mg to about 10 mg, from about 3 mg to about 6 mg, from about 3 mg to about 8 mg, from about 4 mg to about 5 mg, from about 4 mg to about 6 mg, from about 4 mg to about 8 mg, from about 4 mg to about 10 mg, from about 4 mg to about 7 mg, from about 4 mg to about 9 mg, from about 5 mg to about 6 mg, from about 5 mg to about 7 mg, from about 5 mg to about 9 mg, from about 5 mg to about 8 mg, from about 5 mg to about 10 mg, from about 6 mg to about 7 mg, from about 6 mg to about 8 mg, from about 6 mg to about 10 mg, from about 6 mg to about 9 mg, from about 7 mg to about 8 mg, from about 7 mg to about 9 mg, from about 7 mg to about 10 mg, from about 8 mg to about 9 mg, from about 8 mg to about 10 mg, or from about 9 mg to about 10 mg, and any subrange therebetween. In some embodiments, a therapeutically-effective amount of colchicine comprises from about 0.2 mg to about 4 mg, e.g., about 0.3 mg to about 3.6 mg and/or be present in an amount from 0.02% to about 0.4% w/w of the formulation, e.g., about 0.03% to about 0.36% w/w.

The anti-inflammatory or related medicament may be provided at range of amounts of about 10 mg to about 1000 mg, e.g., about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 520 mg, 540 mg, 560 mg, 580 mg, 600 mg, 620 mg, 640 mg, 660 mg, 680 mg, 700 mg, 720 mg, 740 mg, 760 mg, 780 mg, 800 mg, 820 mg, 840 mg, 860 mg, 880 mg, 900 mg, 920 mg, 940 mg, 960 mg, 980 mg, or 1000 mg, or any amount therebetween.

The anti-inflammatory or related medicament may be provided at range of amounts of about 1 g to about 30 g, e.g., about 1 g, 1.1 g, 1.2 g, 1.3 g, 1.4 g, 1.5 g, 1.6 g, 1.7 g, 1.8 g, 1.9 g, 2 g, 2.1 g, 2.2 g, 2.3 g, 2.4 g, 2.5 g, 2.6 g, 2.7 g, 2.8 g, 2.9 g, 3 g, 3.1 g, 3.2 g, 3.3 g, 3.4 g, 3.5 g, 3.6 g, 3.7 g, 3.8 g, 3.9 g, 4 g, 4.1 g, 4.2 g, 4.3 g, 4.4 g, 4.5 g, 4.6 g, 4.7 g, 4.8 g, 4.9 g, 5 g, 5.2 g, 5.4 g, 5.6 g, 5.8 g, 6 g, 6.2 g, 6.4 g, 6.6 g, 6.8 g, 7 g, 7.2 g, 7.4 g, 7.6 g, 7.8 g, 8 g, 8.2 g, 8.4 g, 8.6 g, 8.8 g, 9 g, 9.2 g, 9.4 g, 9.6 g, 9.8 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, or 30 g, or any amount therebetween.

In embodiments, the pharmaceutical composition is orally administered. Such pharmaceutical compositions may be formulated as a liquid, a suspension, a gel, a geltab, a semisolid, a tablet, a sachet, a lozenge, a pill, or a capsule.

In some embodiments, the pharmaceutical composition is parentally administered. The parenteral administration may be via intravenous injection or infusion, intraperitoneal injection, intramuscular injection, or subcutaneous injection.

Pharmaceutical compositions comprising the anti-inflammatory or related medicament may comprise a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, tale, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously or when given as a liquid suspension. Saline solutions and aqueous dextrose and glycerol solutions can also be employed as liquid excipients, specifically for injectable solutions. Suitable pharmaceutical excipients also include starch, glucose (i.e., dextrose), lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any pharmaceutical composition disclosed herein, if desired, can also comprise minor amounts of wetting or emulsifying agents, or pH buffering agents. Examples of suitable pharmaceutical excipients are described in Remington's Pharmaceutical Sciences 1447-1676 (Alfonso R. Gennaro eds., 19th ed. 1995), incorporated herein by reference.

### EMBODIMENTS

It shall be understood that different aspects and/or embodiments of the present disclosure can be appreciated individually, collectively, or in combination with each other. Any description herein concerning a specific formulation, composition, and/or method apply to and may be used for any other specific formulation, composition, and/or method as disclosed herein. Additionally, any formulation or composition disclosed herein is applicable to any herein-disclosed method. In other words, any aspect or embodiment described herein can be combined with any other aspect or embodiment as disclosed herein.

Embodiment A1: A method for treating or reducing inflammation in a subject in need thereof: The method comprising administering to the subject a combination therapy comprising a transdermal formulation comprising a therapeutically-effective amount of a buffering agent and a separate composition comprising a therapeutically-effective amount of colchicine, wherein the colchicine is administered before, contemporaneously with, and/or after administering the transdermal formulation. In this method, the combination therapy reduces blood or serum levels of C-Reactive Protein relative to a subject that is not administered the combination therapy.

Embodiment A2: The method of Embodiment A1, wherein the separate composition is administered orally or topically before, contemporaneously with, and/or after administering the transdermal formulation.

Embodiment A3: The method of Embodiment A1 or Embodiment A2, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A4: The method of any one of Embodiment A1 to Embodiment A3, wherein administering the combination therapy further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

Embodiment A5: A method for treating or reducing inflammation in a subject in need thereof: The method comprising administering to the subject a transdermal formulation comprising a therapeutically-effective amount of a buffering agent and a therapeutically-effective amount of colchicine. In this method, the transdermal formulation reduces blood or serum levels of C-Reactive Protein relative to a subject that is not administered the transdermal formulation.

Embodiment A6: The method of Embodiment A5, wherein the therapeutically-effective amount of colchicine is administered orally or topically before, contemporaneously with, and/or after administering the transdermal formulation.

Embodiment A7: The method of Embodiment A5 or Embodiment A6, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A8: The method of any one of Embodiment A5 to Embodiment A7, wherein administering the transdermal formulation further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

Embodiment A9: A method for preventing inflammation and/or reducing the severity of upcoming inflammation: The method comprising administering to a subject who is not experiencing a symptom of inflammation, a combination therapy comprising a transdermal formulation comprising a therapeutically-effective amount of a buffering agent and a separate composition comprising a therapeutically-effective amount of colchicine, wherein the colchicine is administered before, contemporaneously with, and/or after administering the transdermal formulation. In this method, preventing inflammation and/or reducing the severity of upcoming inflammation is determined in part by a reduction of blood or serum levels of C-Reactive Protein relative to a subject that is not administered the combination therapy.

Embodiment A10: The method of Embodiment A9, wherein the separate composition is administered orally or topically before, contemporaneously with, and/or after administering the transdermal formulation.

Embodiment A11: The method of Embodiment A9 or Embodiment A10, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A12: A method for preventing inflammation and/or reducing the severity of upcoming inflammation: The method comprising administering to a subject who is not experiencing a symptom of inflammation, a transdermal formulation comprising a therapeutically-effective amount of a buffering agent and a therapeutically-effective amount of colchicine. In this method, the transdermal formulation is administered before symptoms inflammation are experienced by the subject and preventing inflammation and/or reducing the severity of upcoming inflammation is determined in part by a reduction of blood or serum levels of C-Reactive Protein relative to a subject that is not administered the transdermal formulation.

Embodiment A13: The method of Embodiment A12, wherein the therapeutically-effective amount of colchicine is administered orally or topically before, contemporaneously with, and/or after administering the transdermal formulation.

Embodiment A14: The method of Embodiment A12 or Embodiment A13, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A15: A method for reducing the likelihood a recurrent inflammation, the method comprising administering to a subject that previously has been treated for inflammation, a combination therapy comprising a transdermal formulation comprising a therapeutically-effective amount of a buffering agent and a separate composition comprising a therapeutically-effective amount of colchicine, wherein the colchicine is administered before, contemporaneously with, and/or after administering the transdermal formulation. In this method, the combination therapy reduces blood or serum levels of C-Reactive Protein relative to a subject that is not administered the combination therapy.

Embodiment A16: The method of Embodiment A15, wherein the separate composition comprising the therapeutically-effective amount of colchicine is administered orally or topically.

Embodiment A17: The method of Embodiment A15 or Embodiment A16, wherein the dosage of the therapeutically-effective amount of the buffering agent is less than the dosage used to previously treat inflammation and the dosage of the therapeutically-effective amount of colchicine is less than the dosage used to previously treat inflammation or the therapeutically-effective amount of colchicine is the same as the dosage used to previously treat inflammation.

Embodiment A18: The method of Embodiment A15 or Embodiment A16, wherein the dosage of the therapeutically-effective amount of the buffering agent is the same as the dosage used to previously treat inflammation and the dosage of the therapeutically-effective amount of colchicine is less than the dosage used to previously treat inflammation or the therapeutically-effective amount of colchicine is the same as the dosage used to previously treat inflammation.

Embodiment A19: The method of any one of Embodiment A15 to Embodiment A18, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A20: A method for reducing the likelihood a recurrent inflammation, the method comprising administering to a subject that previously has been treated for inflammation, a transdermal formulation comprising a therapeutically-effective amount of a buffering agent and a therapeutically-effective amount of colchicine. In this method, the combination therapy reduces blood or serum levels of C-Reactive Protein relative to a subject that is not administered the transdermal formulation.

Embodiment A21: The method of Embodiment A20, wherein the dosage of the therapeutically-effective amount of the buffering agent is less than the dosage used to treat the previous inflammatory event and the dosage of the therapeutically-effective amount of colchicine is less than the dosage used to treat the previous inflammatory event or the therapeutically-effective amount of colchicine is the same as the dosage used to treat the previous inflammatory event.

Embodiment A22: The method of Embodiment A21, wherein the dosage of the therapeutically-effective amount of the buffering agent is the same as the dosage used to treat the previous inflammatory event and the dosage of the therapeutically-effective amount of colchicine is less than the dosage used to treat the previous inflammatory event or the therapeutically-effective amount of colchicine is the same as the dosage used to treat the previous inflammatory event.

Embodiment A23: The method of any one of Embodiment A15 to Embodiment A22, wherein the therapeutically-effective amount of colchicine is administered orally or topically before, contemporaneously with, and/or after administering the transdermal formulation.

Embodiment A24: The method of any one of Embodiment A15 to Embodiment A23, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A25: A method for treating severe pain associated with inflammation, the method comprising administering to a subject having severe pain associated with inflammation a combination therapy comprising administering to the subject a transdermal formulation comprising a therapeutically-effective amount of a buffering agent; administering to the subject a separate composition comprising a therapeutically-effective amount of colchicine; and administering to the subject one of (a) a composition comprising a nonsteroidal medicament or (b) a composition comprising a corticosteroid. In this method, severe pain associated with the inflammation being defined as an ACR score of 7 to 10 and the combination therapy reduces blood or serum levels of C-Reactive Protein relative to a subject that is not administered the combination therapy.

Embodiment A26: The method of Embodiment A25, wherein the separate composition comprising the therapeutically-effective amount of colchicine is administered before, contemporaneously with, and/or after administering the transdermal formulation.

Embodiment A27: The method of Embodiment A25 or Embodiment A26, wherein the transdermal formulation is administered before, contemporaneously with, and/or after (a) the composition comprising the nonsteroidal medicament or (b) the composition comprising the corticosteroid.

Embodiment A28: The method of any one of Embodiment A25 to Embodiment A27, wherein the subject is administered both of (a) the composition comprising the nonsteroidal medicament and (b) the composition comprising the corticosteroid.

Embodiment A29: The method of any one of Embodiment A25 to Embodiment A28, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment A30: The method of any one of Embodiment A25 to Embodiment A28, wherein the separate composition comprising the therapeutically-effective amount of colchicine is administered orally or is administered topically.

Embodiment A31: The method of any one of Embodiment A25 to Embodiment A30, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment A32: A method for treating severe pain associated with inflammation, the method comprising administering to a subject having severe pain associated with inflammation a combination therapy comprising administering to the subject a transdermal formulation comprising: a therapeutically-effective amount of a buffering agent, and a therapeutically-effective amount of colchicine and administering to the subject one or both of (a) a composition comprising a nonsteroidal medicament or (b) a composition comprising a corticosteroid. In this method, severe pain associated with the inflammation being defined as an ACR score of 7 to 10 and the combination therapy reduces blood or serum levels of C-Reactive Protein relative to a subject that is not administered the combination therapy.

Embodiment A33: The method of Embodiment A32, wherein the transdermal formulation is administered before, contemporaneously with, and/or after (a) the composition comprising the nonsteroidal medicament or (b) the composition comprising the corticosteroid.

Embodiment A34: The method of Embodiment A32 or Embodiment A33, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment A35: A method for treating mild to moderate pain associated with inflammation, the method comprising administering to a subject having mild to moderate pain associated with inflammation a combination therapy comprising administering to the subject a transdermal formulation comprising a therapeutically-effective amount of a buffering agent and administering to the subject one of (a) a composition comprising colchicine, (b) a composition comprising a nonsteroidal medicament, or (c) a composition comprising a corticosteroid. In this method, mild pain associated with the inflammation being defined as an ACR score of up to 4 and moderate pain associated with the inflammation being defined as an ACR score of 5 or 6 and the combination therapy reduces blood or serum levels of C-Reactive Protein relative to a subject that is not administered the combination therapy.

Embodiment A36: The method of Embodiment A35, wherein the transdermal formulation is administered before, contemporaneously with, and/or after (a) the composition comprising colchicine, (b) the composition comprising the nonsteroidal medicament, or (c) the composition comprising the corticosteroid.

Embodiment A37: The method of Embodiment A35 or Embodiment A36, wherein the subject is administered two of (a) the composition comprising colchicine, (b) the composition comprising the nonsteroidal medicament, or (c) the composition comprising the corticosteroid.

Embodiment A38: The method of any one of Embodiment A35 to Embodiment A37, wherein the subject is administered each of (a) the composition comprising colchicine, (b) the composition comprising the nonsteroidal medicament, or (c) the composition comprising the corticosteroid.

Embodiment A39: The method of any one of Embodiment A35 to Embodiment A38, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment A40: A method for treating a bone density disorder in a subject in need thereof. The method comprising administering to the subject a combination therapy comprising: a transdermal formulation comprising a therapeutically-effective amount of a buffering agent; and a separate composition comprising a therapeutically-effective amount of colchicine, wherein the colchicine is administered before, contemporaneously with, and/or after administering the transdermal formulation. In this Embodiment, the combination therapy: lowers elevated calcium levels in blood or plasma, stabilizes calcium levels in blood or plasma to levels prior to an inflammatory event, reduces symptoms related to osteoporosis, reduces symptoms related to osteomalacia, improves bone density, and/or inhibits and/or reverses bone decalcification.

Embodiment A41: The method of any one of Embodiment A1 to Embodiment A39, wherein the transdermal formulation comprises a penetrant or penetration enhancer comprising one or more of phosphatidyl choline (e.g., Phospholipon® 90 G), isopropyl palmitate (IPP), steric acid, benzyl alcohol, safflower oil, almond oil, oleic acid, polyglyceryl-4 laurate, poloxamer 407, poloxamer 188, poloxamer 124, menthol, propylene glycol, cetyl alcohol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin.

Embodiment A42: The method of Embodiment A40 or Embodiment A41, wherein the penetrant or penetration enhancer comprises one or more of phosphatidylcholine (e.g., Phospholipon® 90 G), isopropyl palmitate (IPP), isopropyl myristate, stearic acid, benzyl alcohol, ethanol, polyglyceryl-4 laurate, poloxamer 407, and poloxamer 188, poloxamer 124.

Embodiment A43: The method of any one of Embodiment A40 to Embodiment A42, wherein the penetrant or penetration enhancer comprises phosphatidylcholine, hydrogenated phosphatidylcholine, phosphatidylserine, phosphatidylethanolamine, phosphatidylinositol, one or more phosphatides, or one or more inositol phosphatides.

Embodiment A44: The method of any one of Embodiment A40 to Embodiment A43, wherein the penetrant or penetration enhancer comprises from about 3 to about 15% w/w phosphatidylcholine, from about 5 to about 20% w/w isopropyl palmitate, about 1% w/w stearic acid, about 1% w/w benzyl alcohol, from about 1 to about 10% w/w polyglyceryl-4 laurate and from about 5 to about 20% w/w poloxamer 407.

Embodiment A45: The method of any one of Embodiment A40 to Embodiment A44, wherein the penetrant or penetration enhancer comprises benzyl alcohol and/or wherein the penetrant or penetration enhancer comprises a synthetic lecithin.

Embodiment A46: The method of any one of Embodiment A1 to Embodiment A45, wherein the transdermal formulation comprises a source of fatty acids.

Embodiment A47: The method of Embodiment A46, wherein the source of fatty acids comprises one or more of an alkanoic acid, almond oil, caprid acid, diacid, ethyloctadecanoic acid, hexanoic acid, lactic acid, lauric acid, a lecithin, linoelaidic acid, linoleic acid, linolenic acid, macadamia oil, neodecanoic acid, oleic acid, palmitic acid, pelargonic acid, propionic acid, safflower oil, stearic acid, and vaccenic acid, wherein the lecithin is selected from an egg lecithin, a soy lecithin, and a synthetic lecithin.

Embodiment A48: The method of any one of Embodiment A1 to Embodiment A47, wherein the transdermal formulation comprises a polar solvent comprising one or more of ethanol, isopropyl palmitate (IPP), and water.

Embodiment A49: The method of any one of Embodiment A1 to Embodiment A48, wherein the transdermal formulation comprises one or more of a humectant, an emulsifier, a surfactant, and an emollient.

Embodiment A50: The method of Embodiment A49, wherein the emulsifier comprises one or more of cetyl alcohol, Durosoft®, and Phospholipon® 90 G.

Embodiment A51: The method of Embodiment A49 or Embodiment A50, wherein the humectant comprises propylene glycol.

Embodiment A52: The method of any one of Embodiment A49 to Embodiment A51, wherein the surfactant comprises one or more of a poloxamer (e.g., poloxamer 407, poloxamer 188, and poloxamer 124), polyglyceryl-4 laurate, polyoxyethylated castor oil derivative, nonoxynol, octoxynol, phenylsulfonate, a polyoleates, Rewopal®, sodium laurate, sodium lauryl sulfate (sodium dodecyl sulfate), sodium oleate, sorbitan dilaurate, sorbitan dioleate, a sorbitan monolaurate, a sorbitan monooleate; sorbitan trilaurate, sorbitan trioleate, a sorbitan monopalmitate, a sorbitan stearate; a polyethylene glycol, a nonylphenyl ether, p-(1,1,3,3-tetramethylbutyl)-phenyl ether (Triton™ X-100), or a polysorbate (e.g., a Tween®). Embodiment A53: The method of Embodiment A52, wherein the poloxamer is a Pluronic®.

Embodiment A54: The method of any one of Embodiment A1 to Embodiment A53, wherein the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

Embodiment A55: The method of any one of Embodiment A1 to Embodiment A54, wherein the transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

Embodiment A56: The method of Embodiment A54 or Embodiment A55, wherein when colchicine is included in the transdermal formulation, the amount of deionized water is reduced to provide for the addition of the therapeutically-effective amount of colchicine.

Embodiment A57: The method of any one of Embodiment A1 to Embodiment A56, wherein the concentration of the buffering agent is from about 10% to about 50% w/w of the transdermal formulation.

Embodiment A58: The method of any one of Embodiment A1 to Embodiment A57, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

Embodiment A59: The method of any one of Embodiment A1 to Embodiment A58, wherein the sodium bicarbonate or sodium carbonate is at a concentration from about 30% to about 35% w/w of the transdermal formulation, optionally, wherein the sodium bicarbonate or sodium carbonate is at a concentration of about 33% w/w of the transdermal formulation.

Embodiment A60: The method of any one of Embodiment A1 to Embodiment A59, wherein the transdermal formulation comprises menthol, e.g., at a concentration from about 0.1% to about 5.0% w/w of the transdermal formulation.

Embodiment A61: The method of any one of Embodiment A1 to Embodiment A60, wherein the transdermal formulation comprises about 33% w/w sodium bicarbonate or sodium carbonate and about 0.5% w/w menthol.

Embodiment A62: The method of any one of Embodiment A1 to Embodiment A61, wherein the transdermal formulation has a pH from about 9 to about 11 or from about 7 to about 10.5.

Embodiment A63: The method of any one of Embodiment A1 to Embodiment A62, wherein method comprises administering the transdermal formulation about three times a day.

Embodiment A64: The method of any one of Embodiment A1 to Embodiment A63, wherein the transdermal formulation is formulated as a cream, lotion, or ointment.

Embodiment A65: The method of any of one of Embodiment A1 to Embodiment A64 wherein the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

Embodiment A66: The method of any one of Embodiment A1 to Embodiment A65, wherein the therapeutically-effective amount of colchicine comprises from about 0.2 mg to about 4 mg, e.g., about 0.3 mg to about 3.6 mg and/or be present in an amount from 0.02% to about 0.4% w/w of the formulation, e.g., about 0.03% to about 0.36% w/w.

Embodiment A67: The method of any one of Embodiment A1 to Embodiment A66, wherein transdermal formulation is as described in any of Table 1 to Table 19 or as described elsewhere herein.

Embodiment A68: A transdermal formulation for use in method of treating or reducing inflammation in a subject in need thereof: The transdermal formulation comprises a therapeutically-effective amount of a buffering agent and a therapeutically-effective amount of colchicine. This transdermal formulation reduces blood or serum levels of C-Reactive Protein relative to a subject that is not administered the combination therapy.

Embodiment A69: The transdermal formulation of Embodiment A68, wherein the transdermal formulation further reduces or eliminates the need for a rescue medicine; improves the subject's physical function as measured by a Patient-Reported Outcomes Measurement Information System (PROMIS) score, e.g., PROMIS PF 20; provides an improvement in the subject's Sum of Pain Intensity Difference (SPID) score; lowers the subject's pain-numeric rating; decreases the time to resolution of pain relative to a historical control patient; lowers the subject-reported or physician-assessed moderate-to-severe joint tenderness; lowers the subject-reported or physician-assessed moderate-to-severe joint swelling; reduces uric acid crystal levels in blood or plasma; raises urine pH, and/or increases patient satisfaction.

Embodiment A70: A transdermal formulation for use in method of preventing inflammation and/or reducing the severity of upcoming inflammation: The transdermal formulation comprises a therapeutically-effective amount of a buffering agent and comprises a therapeutically-effective amount of colchicine: This transdermal formulation is administered before symptoms of inflammation are experienced by the subject. With this transdermal formulation, preventing inflammation and/or reducing the severity of upcoming inflammation is determined in part by a reduction of blood or serum levels of C-Reactive Protein relative to a subject that is not administered the combination therapy.

Embodiment A71: A transdermal formulation for use in method of reducing the likelihood a recurrent inflammation, the method comprising administering to a subject that previously has been treated for inflammation: The transdermal formulation comprises a therapeutically-effective amount of a buffering agent and comprises a therapeutically-effective amount of colchicine: This transdermal formulation is administered before symptoms of inflammation are experienced by the subject. With this transdermal formulation, reducing the likelihood of a recurrent inflammation is determined in part by a reduction of blood or serum levels of C-Reactive Protein relative to a subject that is not administered the combination therapy.

Embodiment A72: A transdermal formulation for use in method of treating chronic inflammation, the method comprising administering to a subject that previously has been treated for inflammation: The transdermal formulation comprises a therapeutically-effective amount of a buffering agent and comprises a therapeutically-effective amount of colchicine. With this transdermal formulation, treating chronic inflammation is determined in part by a reduction of blood or serum levels of C-Reactive Protein relative to a subject that is not administered the combination therapy.

Embodiment A73: A transdermal formulation for use in method of treating a bone density disorder in a subject in need thereof. The method comprising administering to the subject, the transdermal formulation comprising a therapeutically-effective amount of a buffering agent and comprising a therapeutically-effective amount of colchicine.

Embodiment A74: The transdermal formulation of any one of Embodiment A68 to Embodiment A73, wherein the transdermal formulation comprises a penetrant or penetration enhancer comprising one or more of phosphatidyl choline (e.g., Phospholipon® 90 G), isopropyl palmitate (IPP), steric acid, benzyl alcohol, safflower oil, almond oil, oleic acid, polyglyceryl-4 laurate, poloxamer 407, poloxamer 188, poloxamer 124, menthol, propylene glycol, cetyl alcohol, ethanol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin.

Embodiment A75: The transdermal formulation of Embodiment A73 or Embodiment A74, wherein the penetrant or penetration enhancer comprises one or more of phosphatidylcholine (e.g., Phospholipon® 90 G), isopropyl palmitate (IPP), stearic acid, benzyl alcohol, polyglyceryl-4 laurate, poloxamer 407 poloxamer 188, or poloxamer 124.

Embodiment A76: The transdermal formulation of any one of Embodiment A73 to Embodiment A75 wherein the penetrant or penetration enhancer comprises phosphatidylcholine, hydrogenated phosphatidylcholine, phosphatidylserine, phosphatidylethanolamine, phosphatidylinositol, one or more phosphatides, or one or more inositol phosphatides.

Embodiment A77: The transdermal formulation of any one of Embodiment A73 to Embodiment A75, wherein the penetrant or penetration enhancer comprises from about 3 to about 15% w/w phosphatidylcholine, from about 5 to about 20% w/w isopropyl palmitate, about 1% w/w stearic acid, about 1% w/w benzyl alcohol, from about 1 to about 10% w/w polyglyceryl-4 laurate and from about 5 to about 20% w/w poloxamer 407.

Embodiment A78: The transdermal formulation of any one of Embodiment A73 to Embodiment A77, wherein the penetrant or penetration enhancer comprises benzyl alcohol and/or wherein the penetrant or penetration enhancer comprises a synthetic lecithin.

Embodiment A79: The transdermal formulation of any one of Embodiment A68 to Embodiment A78, wherein the transdermal formulation comprises a source of fatty acids.

Embodiment A80: The transdermal formulation of Embodiment A79, wherein the source of fatty acids comprises one or more of an alkanoic acid, almond oil, caprid acid, diacid, ethyloctadecanoic acid, hexanoic acid, lactic acid, lauric acid, a lecithin, linoelaidic acid, linoleic acid, linolenic acid, neodecanoic acid, oleic acid, palmitic acid, pelargonic acid, propionic acid, safflower oil, stearic acid, and vaccenic acid, wherein the lecithin is selected from an egg lecithin, a soy lecithin, and a synthetic lecithin.

Embodiment A81: The transdermal formulation of any one of Embodiment A68 to Embodiment A80, wherein the transdermal formulation comprises a polar solvent comprising one or more of ethanol, isopropyl palmitate (IPP), and water.

Embodiment A82: The transdermal formulation of any one of Embodiment A68 to Embodiment A81, wherein the transdermal formulation comprises one or more of a humectant, an emulsifier, a surfactant, and an emollient.

Embodiment A83: The transdermal formulation of Embodiment A82, wherein the emulsifier comprises one or more of cetyl alcohol, Durosoft®, and Phospholipon® 90 G.

Embodiment A84: The transdermal formulation of Embodiment A82 or Embodiment A83, wherein the humectant comprises propylene glycol.

Embodiment A85: The transdermal formulation of any one of Embodiment A82 to Embodiment A84, wherein the surfactant comprises one or more of a poloxamer (e.g., poloxamer 407, poloxamer 188, and poloxamer 124), polyglyceryl-4 laurate, polyoxyethylated castor oil derivative, nonoxynol, octoxynol, phenylsulfonate, a polyoleates, Rewopal®, sodium laurate, sodium lauryl sulfate (sodium dodecyl sulfate), sodium oleate, sorbitan dilaurate, sorbitan dioleate, a sorbitan monolaurate, a sorbitan monooleate; sorbitan trilaurate, sorbitan trioleate, a sorbitan monopalmitate, a sorbitan stearate; a polyethylene glycol, a nonylphenyl ether, p-(1,1,3,3-tetramethylbutyl)-phenyl ether (Triton™ X-100), or a polysorbate (e.g., a Tween®).

Embodiment A86: The transdermal formulation of Embodiment A85, wherein the poloxamer is a Pluronic®.

Embodiment A87: The transdermal formulation of any one of Embodiment A68 to Embodiment A86, wherein the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

Embodiment A88: The transdermal formulation of any one of Embodiment A68 to Embodiment A87, wherein the transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

Embodiment A89: The transdermal formulation of Embodiment A87 or Embodiment A88, wherein when colchicine is included in the transdermal formulation, the amount of deionized water is reduced to provide for the addition of the therapeutically-effective amount of colchicine.

Embodiment A90: The transdermal formulation of any one of Embodiment A68 to Embodiment A89, wherein the concentration of the buffering agent is from about 10% to about 50% w/w of the transdermal formulation.

Embodiment A91: The transdermal formulation of any one of Embodiment A68 to Embodiment A90, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

Embodiment A92: The transdermal formulation of any one of Embodiment A68 to Embodiment A90, wherein the sodium bicarbonate or sodium carbonate is at a concentration from about 30% to about 35% w/w of the transdermal formulation, e.g., the sodium bicarbonate or sodium carbonate is at a concentration of about 33% w/w of the transdermal formulation.

Embodiment A92: The transdermal formulation of any one of Embodiment A67 to Embodiment A91, wherein the transdermal formulation comprises menthol, e.g., at a concentration from about 0.1% to about 5.0% w/w of the transdermal formulation.

Embodiment A94: The transdermal formulation of any one of Embodiment A67 to Embodiment A93, wherein the transdermal formulation comprises about 33% w/w sodium bicarbonate or sodium carbonate and about 0.5% w/w menthol.

Embodiment A95: The transdermal formulation of any one of Embodiment A67 to Embodiment A94, wherein the transdermal formulation has a pH from about 9 to about 11.

Embodiment A96: The transdermal formulation of any one of Embodiment A67 to Embodiment A95, wherein the transdermal formulation has a pH from about 7 to about 10.5.

Embodiment A97: The transdermal formulation of any one of Embodiment A67 to Embodiment A96, wherein the transdermal formulation is formulated as a cream, lotion, or ointment.

Embodiment A98: The transdermal formulation of any one of Embodiment A67 to Embodiment A97, wherein the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

Embodiment A99: The transdermal formulation of any one of Embodiment A67 to Embodiment A98, wherein the therapeutically-effective amount of colchicine comprises from about 0.2 mg to about 4 mg, e.g., about 0.3 mg to about 3.6 mg and/or be present in an amount from 0.02% to about 0.4% w/w of the formulation, e.g., about 0.03% to about 0.36% w/w.

Embodiment A100: The transdermal formulation of any one of Embodiment A67 to Embodiment A99, wherein transdermal formulation is as described in any of Table 1 to Table 19 or as described elsewhere herein.

Embodiment A101: A plurality of formulations comprising the transdermal formulation of any one of Embodiment A68 to Embodiment A100 and a second composition comprising a nonsteroidal medicament.

Embodiment A102: The plurality of formulations of Embodiment A101, wherein the nonsteroidal medicament is a nonsteroidal anti-inflammatory drug (NSAID), a COX-2 Inhibitor, an Opioid, and/or Illaris (canakinumab).

Embodiment A103: A plurality of formulations comprising the transdermal formulation of any one of Embodiment A68 to Embodiment A100 and a second composition comprising a corticosteroid or colchicine.

Embodiment A104: The plurality of formulations of Embodiment A103, wherein, the corticosteroid is one or more of prednisone, methylprednisolone, prednisolone, triamcinolone, and/or a glucocorticoid.

Embodiment A105: The plurality of formulations of any one of Embodiment A101 to Embodiment A104, wherein the second composition is administered orally or topically before, contemporaneously with, and/or after administering the transdermal formulation.

Embodiment B1: A method for treating or reducing inflammation in a subject in need thereof: The method comprising administering to the subject a transdermal formulation comprising a therapeutically-effective amount of a buffering agent. In this method, the transdermal formulation reduces blood or serum levels of C-Reactive Protein relative to a subject that is not administered the combination therapy.

Embodiment B2: The method of Embodiment B1, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B3: The method of Embodiment B1 or Embodiment B2, wherein administering the transdermal formulation further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

Embodiment B4: A method for treating or reducing inflammation in a subject in need thereof: The method comprises administering to the subject a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy.

Embodiment B5: The method of Embodiment B4, wherein the separate composition is administered orally or topically before, contemporaneously with, and/or after administering the transdermal formulation.

Embodiment B6: The method of Embodiment B4 or Embodiment B5, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B7: The method of any one of Embodiment B4 to Embodiment B6, wherein administering the combination therapy further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

Embodiment B8: The method of any one of Embodiment B4 to Embodiment B7, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B9: A method for treating or reducing inflammation in a subject in need thereof, the method comprising administering to the subject a transdermal formulation comprises a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

Embodiment B10: The method of Embodiment B9, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B11: The method of Embodiment B9 or Embodiment B10, wherein administering the transdermal formulation further reduces or eliminates the need for a rescue medicine, improves the subject's physical function as measured by a Patient-Reported Outcomes Measurement Information System (PROMIS) score, e.g., PROMIS PF 20, provides an improvement in the subject's Sum of Pain Intensity Difference (SPID) score, lowers the subject's pain-numeric rating, decreases the time to resolution of pain relative to a historical control patient, lowers the subject-reported or physician-assessed moderate-to-severe joint tenderness, lowers the subject-reported or physician-assessed moderate-to-severe joint swelling, reduces uric acid crystal levels in blood or plasma, raises urine pH, lowers elevated calcium levels in blood or plasma, stabilizes calcium levels in blood or plasma to levels prior to an inflammatory event, reduces symptoms related to osteoporosis, reduces symptoms related to osteomalacia, improves bone density, inhibits and/or reverses bone decalcification, and/or increases patient satisfaction.

Embodiment B12: The method of any one of Embodiment B9 to Embodiment B11, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B13: A method for preventing inflammation and/or reducing the severity of upcoming inflammation, the method comprising administering to a subject who is not experiencing a symptom of inflammation a transdermal formulation comprising a therapeutically-effective amount of a buffering agent. In this method, the transdermal formulation reduces blood or serum levels of C-Reactive Protein relative to a subject that is not administered the combination therapy.

Embodiment B14: The method of Embodiment B13, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B15: A method for preventing inflammation and/or reducing the severity of upcoming inflammation, the method comprising administering to a subject who is not experiencing a symptom of inflammation, a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy Embodiment B16: The method of Embodiment B15, wherein the separate composition is administered orally or topically before, contemporaneously with, and/or after administering the transdermal formulation.

Embodiment B17: The method of Embodiment B15 or Embodiment B16, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B18: The method of any one of Embodiment B15 to Embodiment B17, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B19: A method for preventing inflammation and/or reducing the severity of upcoming inflammation, the method comprising administering to a subject who is not experiencing a symptom of inflammation, a transdermal formulation comprising a therapeutically-effective amount of a buffering agent and a therapeutically-effective amount of a nonsteroidal medicament and/or a corticosteroid. In this method, the transdermal formulation is administered before symptoms inflammation are experienced by the subject and prevents inflammation and/or reduces the severity of upcoming inflammation is determined in part by a reduction of blood or serum levels of C-Reactive Protein relative to a subject that is not administered the combination therapy.

Embodiment B20: The method of Embodiment B19, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B21: The method of Embodiment B19 or Embodiment B20, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B22: A method for reducing the likelihood a recurrent inflammation, the method comprising administering to a subject that previously has been treated for inflammation, a transdermal formulation comprising a therapeutically-effective amount of a buffering agent. In this method, the transdermal formulation reduces blood or serum levels of C-Reactive Protein relative to a subject that is not administered the combination therapy.

Embodiment B23: The method of Embodiment B22, wherein the dosage of the therapeutically-effective amount of the buffering agent is less than the dosage used to previously treat inflammation.

Embodiment B24: The method of Embodiment B22, wherein the dosage of the therapeutically-effective amount of the buffering agent is the same as the dosage used to previously treat inflammation.

Embodiment B25: The method of any one of Embodiment B22 to Embodiment B24, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B26: A method for reducing the likelihood a recurrent inflammation, the method comprising administering to a subject that previously has been treated for inflammation, a transdermal formulation comprising a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

Embodiment B27: The method of Embodiment B26, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B28: The method of Embodiment B26, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B29: The method of any one of Embodiment B26 to Embodiment B28, wherein another therapeutically-effective amount of the nonsteroidal medicament and/or the corticosteroid is administered orally or topically before, contemporaneously with, and/or after administering the transdermal formulation.

Embodiment B30: The method of any one of Embodiment B26 to Embodiment B29, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B31: The method of any one of Embodiment B26 to Embodiment B30, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B32: A method for reducing the likelihood a recurrent inflammation, the method comprising administering to a subject that previously has been treated for inflammation, administering to the subject a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the transdermal formulation reduces blood or serum levels of C-Reactive Protein relative to a subject that is not administered the combination therapy.

Embodiment B33: The method of Embodiment B32, wherein the separate composition is administered orally or topically before, contemporaneously with, and/or after administering the transdermal formulation.

Embodiment B34: The method of Embodiment B32 or Embodiment B33, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B35: The method of Embodiment B32 or Embodiment B33, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B36: The method of any one of Embodiment B32 to Embodiment B35, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B37: The method of any one of Embodiment B32 to Embodiment B36, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B38: A method for treating chronic inflammation, the method comprising administering to a subject that previously has been treated for inflammation, a transdermal formulation comprising a therapeutically-effective amount of a buffering agent. In this method, the transdermal formulation reduces blood or serum levels of C-Reactive Protein relative to a subject that is not administered the combination therapy.

Embodiment B39: The method of Embodiment B38, wherein the dosage of the therapeutically-effective amount of the buffering agent is less than the dosage used to previously treat inflammation.

Embodiment B40: The method of Embodiment B38, wherein the dosage of the therapeutically-effective amount of the buffering agent is the same as the dosage used to previously treat inflammation.

Embodiment B41: The method of any one of Embodiment B38 to Embodiment B40, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B42: A method for treating chronic inflammation, the method comprising administering to a subject that previously has been treated for inflammation, a transdermal formulation comprising a therapeutically-effective amount of a buffering agent and a therapeutically-effective amount of a nonsteroidal medicament and/or a corticosteroid. In this method, the transdermal formulation reduces blood or serum levels of C-Reactive Protein relative to a subject that is not administered the combination therapy.

Embodiment B43: The method of Embodiment B42, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B44: The method of Embodiment B42, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B45: The method of any one of Embodiment B42 to Embodiment B44, wherein another therapeutically-effective amount of the nonsteroidal medicament and/or the corticosteroid is administered orally or topically before, contemporaneously with, and/or after administering the transdermal formulation.

Embodiment B46: The method of any one of Embodiment B42 to Embodiment B45, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B47: The method of any one of Embodiment B42 to Embodiment B46, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B48: A method for treating chronic inflammation, the method comprising administering to a subject that previously has been treated for inflammation, a combination therapy comprising a transdermal formulation comprising a therapeutically-effective amount of a buffering agent and a separate composition comprising a therapeutically-effective amount of a nonsteroidal medicament and/or a corticosteroid. In this method, the separate composition is administered before, contemporaneously with, and/or after administering the transdermal formulation and the combination therapy reduces blood or serum levels of C-Reactive Protein relative to a subject that is not administered the combination therapy.

Embodiment B49: The method of Embodiment B48, wherein the separate composition is administered orally or topically before, contemporaneously with, and/or after administering the transdermal formulation.

Embodiment B50: The method of Embodiment B48 or Embodiment B49, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B51: The method of Embodiment B48 or Embodiment B49, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

Embodiment B52: The method of any one of Embodiment B48 to Embodiment B51, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

Embodiment B53: The method of any one of Embodiment B48 to Embodiment B52, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B54: A method for treating a bone density disorder in a subject in need thereof, the method comprising administering to the subject a transdermal formulation comprising a therapeutically-effective amount of a buffering agent. In this method, the transdermal formulation lowers elevated calcium levels in blood or plasma, stabilizes calcium levels in blood or plasma to levels prior to an inflammatory event, reduces symptoms related to osteoporosis, reduces symptoms related to osteomalacia, improves bone density, and/or inhibits and/or reverses bone decalcification.

Embodiment B55: The method of any one of Embodiment B1 to Embodiment B54, wherein the transdermal formulation comprises a penetrant or penetration enhancer comprising one or more of phosphatidyl choline (e.g., Phospholipon® 90 G), isopropyl palmitate (IPP), steric acid, benzyl alcohol, safflower oil, almond oil, oleic acid, polyglyceryl-4 laurate, poloxamer 407, poloxamer 188, poloxamer 124, menthol, propylene glycol, cetyl alcohol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin.

Embodiment B56: The method of Embodiment B54 or Embodiment B55, wherein the penetrant or penetration enhancer comprises one or more of phosphatidylcholine (e.g., Phospholipon® 90 G), isopropyl palmitate (IPP), isopropyl myristate, stearic acid, benzyl alcohol, ethanol, polyglyceryl-4 laurate, poloxamer 407, and poloxamer 188, poloxamer 124.

Embodiment B57: The method of any one of Embodiment B54 to Embodiment B56, wherein the penetrant or penetration enhancer comprises phosphatidylcholine, hydrogenated phosphatidylcholine, phosphatidylserine, phosphatidylethanolamine, phosphatidylinositol, one or more phosphatides, or one or more inositol phosphatides.

Embodiment B58: The method of any one of Embodiment B5 to Embodiment B57, wherein the penetrant or penetration enhancer comprises from about 3 to about 15% w/w phosphatidylcholine, from about 5 to about 20% w/w isopropyl palmitate, about 1% w/w stearic acid, about 1% w/w benzyl alcohol, from about 1 to about 10% w/w polyglyceryl-4 laurate and from about 5 to about 20% w/w poloxamer 407.

Embodiment B59: The method of any one of Embodiment B54 to Embodiment B58, wherein the penetrant or penetration enhancer comprises benzyl alcohol and/or wherein the penetrant or penetration enhancer comprises a synthetic lecithin.

Embodiment B60: The method of any one of Embodiment B1 to Embodiment B59, wherein the transdermal formulation comprises a source of fatty acids.

Embodiment B61: The method of Embodiment B60, wherein the source of fatty acids comprises one or more of an alkanoic acid, almond oil, caprid acid, diacid, ethyloctadecanoic acid, hexanoic acid, lactic acid, lauric acid, a lecithin, linoelaidic acid, linoleic acid, linolenic acid, macadamia oil, neodecanoic acid, oleic acid, palmitic acid, pelargonic acid, propionic acid, safflower oil, stearic acid, and vaccenic acid, wherein the lecithin is selected from an egg lecithin, a soy lecithin, and a synthetic lecithin.

Embodiment B62: The method of any one of Embodiment B1 to Embodiment B61, wherein the transdermal formulation comprises a polar solvent comprising one or more of ethanol, isopropyl palmitate (IPP), and water.

Embodiment B63: The method of any one of Embodiment B1 to Embodiment B62, wherein the transdermal formulation comprises one or more of a humectant, an emulsifier, a surfactant, and an emollient.

Embodiment B64: The method of Embodiment B63, wherein the emulsifier comprises one or more of cetyl alcohol, Durosoft®, and Phospholipon® 90 G.

Embodiment B65: The method of Embodiment B63 or Embodiment B64, wherein the humectant comprises propylene glycol.

Embodiment B66: The method of any one of Embodiment B63 to Embodiment B65, wherein the surfactant comprises one or more of a poloxamer (e.g., poloxamer 407, poloxamer 188, and poloxamer 124), polyglyceryl-4 laurate, polyoxyethylated castor oil derivative, nonoxynol, octoxynol, phenylsulfonate, a polyoleates, Rewopal®, sodium laurate, sodium lauryl sulfate (sodium dodecyl sulfate), sodium oleate, sorbitan dilaurate, sorbitan dioleate, a sorbitan monolaurate, a sorbitan monooleate; sorbitan trilaurate, sorbitan trioleate, a sorbitan monopalmitate, a sorbitan stearate; a polyethylene glycol, a nonylphenyl ether, p-(1,1,3,3-tetramethylbutyl)-phenyl ether (Triton™ X-100), or a polysorbate (e.g., a Tween®).

Embodiment B67: The method of Embodiment B66, wherein the poloxamer is a Pluronic®.

Embodiment B68: The method of any one of Embodiment B1 to Embodiment B67, wherein the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

Embodiment B69: The method of any one of Embodiment B1 to Embodiment B68, wherein the transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

Embodiment B70: The method of Embodiment B68 or Embodiment B69, wherein when the nonsteroidal medicament and/or the corticosteroid is included in the transdermal formulation, the amount of deionized water is reduced to provide for the addition of the therapeutically-effective amount of the nonsteroidal medicament and/or the corticosteroid.

Embodiment B71: The method of any one of Embodiment B1 to Embodiment B70, wherein the concentration of the buffering agent is from about 10% to about 50% w/w of the transdermal formulation Embodiment B72: The method of any one of Embodiment B1 to Embodiment B57, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

Embodiment B73: The method of any one of Embodiment B1 to Embodiment B72, wherein the sodium bicarbonate or sodium carbonate is at a concentration from about 30% to about 35% w/w of the transdermal formulation, e.g., the sodium bicarbonate or sodium carbonate is at a concentration of about 33% w/w of the transdermal formulation.

Embodiment B74: The method of any one of Embodiment B1 to Embodiment B73, wherein the transdermal formulation comprises menthol, e.g., at a concentration from about 0.1% to about 5.0% w/w of the transdermal formulation.

Embodiment B75: The method of any one of Embodiment B1 to Embodiment B74, wherein the transdermal formulation comprises about 33% w/w sodium bicarbonate or sodium carbonate and about 0.5% w/w menthol.

Embodiment B76: The method of any one of Embodiment B1 to Embodiment B75, wherein the transdermal formulation has a pH from about 9 to about 11 or from about 7 to about 10.5.

Embodiment B77: The method of any one of Embodiment B1 to Embodiment B76, wherein method comprises administering the transdermal formulation about three times a day.

Embodiment B78: The method of any one of Embodiment B1 to Embodiment B77, wherein the transdermal formulation is formulated as a cream, lotion, or ointment.

Embodiment B79: The method of any of one of Embodiment B1 to Embodiment B78 wherein the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

Embodiment B80: The method of any one of Embodiment B1 to Embodiment B79, wherein transdermal formulation is as described in any of Table 1 to Table 19 or as described elsewhere herein.

Embodiment B81: A transdermal formulation for use in method of treating or reducing inflammation in a subject in need thereof, the transdermal formulation comprising a therapeutically-effective amount of a buffering agent and comprising a therapeutically-effective amount of a nonsteroidal medicament and/or a corticosteroid: The transdermal formulation reduces blood or serum levels of C-Reactive Protein relative to a subject that is not administered the combination therapy.

Embodiment B82: The transdermal formulation of Embodiment B81, wherein the transdermal formulation further reduces or eliminates the need for a rescue medicine; improves the subject's physical function as measured by a Patient-Reported Outcomes Measurement Information System (PROMIS) score, e.g., PROMIS PF 20; provides an improvement in the subject's Sum of Pain Intensity Difference (SPID) score; lowers the subject's pain-numeric rating; decreases the time to resolution of pain relative to a historical control patient; lowers the subject-reported or physician-assessed moderate-to-severe joint tenderness; lowers the subject-reported or physician-assessed moderate-to-severe joint swelling; reduces uric acid crystal levels in blood or plasma; raises urine pH, and/or increases patient satisfaction.

Embodiment B83: A transdermal formulation for use in method of preventing inflammation and/or reducing the severity of upcoming inflammation, the transdermal formulation comprising a therapeutically-effective amount of a buffering agent and comprising a therapeutically-effective amount of a nonsteroidal medicament and/or a corticosteroid: The transdermal formulation is administered before symptoms of inflammation are experienced by the subject. The transdermal formulation prevents inflammation and/or reduces the severity of upcoming inflammation is determined in part by a reduction of blood or serum levels of C-Reactive Protein relative to a subject that is not administered the combination therapy.

Embodiment B84: A transdermal formulation for use in method of reducing the likelihood a recurrent inflammation, the method comprising administering to a subject that previously has been treated for inflammation, the transdermal formulation comprising a therapeutically-effective amount of a buffering agent, and comprising a therapeutically-effective amount of a nonsteroidal medicament and/or a corticosteroid The transdermal formulation is administered before symptoms of inflammation are experienced by the subject and reduces the likelihood of a recurrent inflammation is determined in part by a reduction of blood or serum levels of C-Reactive Protein relative to a subject that is not administered the combination therapy.

Embodiment B85: A transdermal formulation for use in method of treating chronic inflammation, the method comprising administering to a subject that previously has been treated for inflammation, the transdermal formulation comprising a therapeutically-effective amount of a buffering agent and comprising a therapeutically-effective amount of a nonsteroidal medicament and/or a corticosteroid. Here, treating chronic inflammation is determined in part by a reduction of blood or serum levels of C-Reactive Protein relative to a subject that is not administered the combination therapy.

Embodiment B86: The transdermal formulation of any one of Embodiment B81 to Embodiment B85, further comprising a therapeutically-effective amount of a nonsteroidal medicament and/or a corticosteroid.

Embodiment B87: The transdermal formulation of Embodiment B86, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Embodiment B88: A transdermal formulation for use in method of treating a bone density disorder in a subject in need thereof. The method comprising administering to the subject, the transdermal formulation comprising a therapeutically-effective amount of a buffering agent.

Embodiment B89: The transdermal formulation of any one of Embodiment B81 to Embodiment B86, wherein the transdermal formulation comprises a penetrant or penetration enhancer comprising one or more of phosphatidyl choline (e.g., Phospholipon® 90 G), isopropyl palmitate (IPP), steric acid, benzyl alcohol, safflower oil, almond oil, oleic acid, polyglyceryl-4 laurate, poloxamer 407, poloxamer 188, poloxamer 124, menthol, propylene alcohol, cetyl alcohol, ethanol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin.

Embodiment B90: The transdermal formulation of Embodiment B88 or Embodiment B89, wherein the penetrant or penetration enhancer comprises one or more of phosphatidylcholine (e.g., Phospholipon® 90 G), isopropyl palmitate (IPP), stearic acid, benzyl alcohol, polyglyceryl-4 laurate, poloxamer 407 poloxamer 188, or poloxamer 124.

Embodiment B91: The transdermal formulation of any one of Embodiment B88 to Embodiment B90, wherein the penetrant or penetration enhancer comprises phosphatidylcholine, hydrogenated phosphatidylcholine, phosphatidylserine, phosphatidylethanolamine, phosphatidylinositol, one or more phosphatides, or one or more inositol phosphatides.

Embodiment B92: The transdermal formulation of any one of Embodiment B88 to Embodiment B91, wherein the penetrant or penetration enhancer comprises from about 3 to about 15% w/w phosphatidylcholine, from about 5 to about 20% w/w isopropyl palmitate, about 1% w/w stearic acid, about 1% w/w benzyl alcohol, from about 1 to about 10% w/w polyglyceryl-4 laurate and from about 5 to about 20% w/w poloxamer 407.

Embodiment B93: The transdermal formulation of any one of Embodiment B88 to Embodiment B92, wherein the penetrant or penetration enhancer comprises benzyl alcohol and/or wherein the penetrant or penetration enhancer comprises a synthetic lecithin.

Embodiment B94: The transdermal formulation of any one of Embodiment B81 to Embodiment B93, wherein the transdermal formulation comprises a source of fatty acids.

Embodiment B95: The transdermal formulation of Embodiment B94, wherein the source of fatty acids comprises one or more of an alkanoic acid, almond oil, caprid acid, diacid, ethyloctadecanoic acid, hexanoic acid, lactic acid, lauric acid, a lecithin, linoelaidic acid, linoleic acid, linolenic acid, neodecanoic acid, oleic acid, palmitic acid, pelargonic acid, propionic acid, safflower oil, stearic acid, and vaccenic acid, wherein the lecithin is selected from an egg lecithin, a soy lecithin, and a synthetic lecithin.

Embodiment B96: The transdermal formulation of any one of Embodiment B81 to Embodiment B95, wherein the transdermal formulation comprises a polar solvent comprising one or more of ethanol, isopropyl palmitate (IPP), and water.

Embodiment B97: The transdermal formulation of any one of Embodiment B81 to Embodiment B96, wherein the transdermal formulation comprises one or more of a humectant, an emulsifier, a surfactant, and an emollient.

Embodiment B98: The transdermal formulation of Embodiment B97, wherein the emulsifier comprises one or more of cetyl alcohol, Durosoft®, and Phospholipon® 90 G.

Embodiment B99: The transdermal formulation of Embodiment B97 or Embodiment B98, wherein the humectant comprises propylene glycol.

Embodiment B100: The transdermal formulation of any one of Embodiment B97 to Embodiment B99, wherein the surfactant comprises one or more of a poloxamer (e.g., poloxamer 407, poloxamer 188, and poloxamer 124), polyglyceryl-4 laurate, polyoxyethylated castor oil derivative, nonoxynol, octoxynol, phenylsulfonate, a polyoleates, Rewopal®, sodium laurate, sodium lauryl sulfate (sodium dodecyl sulfate), sodium oleate, sorbitan dilaurate, sorbitan dioleate, a sorbitan monolaurate, a sorbitan monooleate; sorbitan trilaurate, sorbitan trioleate, a sorbitan monopalmitate, a sorbitan stearate; a polyethylene glycol, a nonylphenyl ether, p-(1,1,3,3-tetramethylbutyl)-phenyl ether (Triton™ X-100), or a polysorbate (e.g., a Tween®).

Embodiment B101: The transdermal formulation of Embodiment B100, wherein the poloxamer is a Pluronic®.

Embodiment B102: The transdermal formulation of any one of Embodiment B81 to Embodiment B101, wherein the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

Embodiment B103: The transdermal formulation of any one of Embodiment B81 to Embodiment B102, wherein the transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

Embodiment B104: The transdermal formulation of Embodiment B102 or Embodiment B103, wherein when the nonsteroidal medicament and/or the corticosteroid is included in the transdermal formulation, the amount of deionized water is reduced to provide for the addition of the therapeutically-effective amount of the nonsteroidal medicament and/or the corticosteroid.

Embodiment B105: The transdermal formulation of any one of Embodiment B81 to Embodiment B104, wherein the concentration of the buffering agent is from about 10% to about 50% w/w of the transdermal formulation.

Embodiment B106: The transdermal formulation of any one of Embodiment B81 to Embodiment B105, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

Embodiment B107: The transdermal formulation of any one of Embodiment B81 to Embodiment B106, wherein the sodium bicarbonate or sodium carbonate is at a concentration from about 30% to about 35% w/w of the transdermal formulation, e.g., the sodium bicarbonate or sodium carbonate is at a concentration of about 33% w/w of the transdermal formulation.

Embodiment B108: The transdermal formulation of any one of Embodiment B81 to Embodiment B107, wherein the transdermal formulation comprises menthol.

Embodiment B109: The transdermal formulation of Embodiment B108, wherein the menthol is at a concentration from about 0.1% to about 5.0% w/w of the transdermal formulation.

Embodiment B110: The transdermal formulation of any one of Embodiment B81 to Embodiment B109, wherein the transdermal formulation comprises about 33% w/w sodium bicarbonate or sodium carbonate and about 0.5% w/w menthol.

Embodiment B111: The transdermal formulation of any one of Embodiment B81 to Embodiment B110, wherein the transdermal formulation has a pH from about 9 to about 11 or from about 7 to about 10.5.

Embodiment B112: The transdermal formulation of any one of Embodiment B81 to Embodiment B111, wherein the transdermal formulation is formulated as a cream, lotion, or ointment.

Embodiment B113: The transdermal formulation of any one of Embodiment B81 to Embodiment B112, wherein the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

Embodiment B114: The transdermal formulation of any one of Embodiment B81 to Embodiment B113, wherein transdermal formulation is as described in any of Table 1 to Table 19 or as described elsewhere herein.

Embodiment B115: A plurality of formulations comprising the transdermal formulation of any one of Embodiment B81 to Embodiment B114 and a second composition comprising a nonsteroidal medicament.

Embodiment B116: The plurality of formulations of Embodiment B115, wherein the nonsteroidal medicament is a nonsteroidal anti-inflammatory drug (NSAID), a COX-2 Inhibitor, an Opioid, and/or Illaris (canakinumab) Embodiment B117: A plurality of formulations comprising the transdermal formulation of any one of Embodiment B81 to Embodiment B114 and a second composition comprising a corticosteroid or colchicine.

Embodiment B118: The plurality of formulations of Embodiment B116, wherein, the corticosteroid is one or more of prednisone, methylprednisolone, prednisolone, triamcinolone, and/or a glucocorticoid.

Embodiment B119: The plurality of formulations of any one of Embodiment B115 to Embodiment B118, wherein the second composition is administered orally or topically before, contemporaneously with, and/or after administering the transdermal formulation.

### Definitions

The terminology used herein is for the purpose of describing particular cases only and is not intended to be limiting. The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.” By preventing is meant, at least, avoiding the occurrence of a disease and/or reducing the likelihood of acquiring the disease. By treating is meant, at least, ameliorating or avoiding the effects of a disease, including reducing a sign or symptom of the disease.

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

By “one or more” is meant at least one, e.g., one, two, three, four, five, six, seven, eight, nine, ten or more.

A weight percent listed herein may be weight/weight (“w/w”) or weight/volume (“w/v”). In cases, where a weight percent is listed as w/w, it shall be understood that the listed weigh percent is also measured by w/v. For convenience, the present disclosure lists w/w primarily throughout this disclosure, but the recitation of “w/w” means “w/w or w/v”.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but no other embodiments/aspects. Embodiment and aspect can in certain instances be used interchangeably.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human. In some cases, the subject is experiencing inflammation or a symptom thereof. In some cases, the subject has previously experienced inflammation or a symptom thereof but is not presently inflammation or a symptom thereof. In some cases, the subject has chronic inflammation. In some cases, the subject has mild to moderate or severe pain associated with inflammation or a symptom. In some cases, the subject has a bone density disorder or is at risk for a bone density disorder; in these subjects the formulations and methods: lower elevated calcium levels in blood or plasma, stabilize calcium levels in blood or plasma to levels prior to an inflammatory event, reduce symptoms related to osteoporosis, reduce symptoms related to osteomalacia, improve bone density, and/or inhibit and/or reverses bone decalcification. In some cases, the subject has Familial Mediterranean Fever. In some cases, the subject has another joint disease with an inflammatory component, e.g., rheumatoid arthritis.

A subject may have kidney impairment. It is well established that colchicine can cause direct toxicity of the kidneys; thus, colchicine use is counter indicated for subjects with kidney impairment. However, in some aspects and embodiments, the formulations, do not comprise colchicine and can be administered to a subject having kidney impairment, e.g., due to diabetes, chronic kidney disease (CKD), Polycystic kidney disease (PKD), Lupus nephritis, kidney cancer, Alport syndrome, amyloidosis, Goodpasture syndrome, and Wegener's granulomatosis, and/or recipients of a renal transplant.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” or “a therapeutically-effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin or muscles.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

Many known and useful compounds and the like can be found in Remington's Pharmaceutical Sciences (13^{th} Ed), Mack Publishing Company, Easton, PA-a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Any aspect or embodiment described herein can be combined with any other aspect or embodiment as disclosed herein.

### EXAMPLES

The following examples are given for the purpose of illustrating various embodiments of the disclosure and are not meant to limit the present disclosure in any fashion. The present examples, along with the methods described herein are presently representative of preferred embodiments, are illustrative, and are not intended as limitations on the scope of the disclosure. Changes therein and other uses which are encompassed within the spirit of the disclosure as defined by the scope of the claims will occur to those skilled in the art.

### Example 1: C-Reactive Protein is Reduced by Transdermal Formulations and Methods of the Present Disclosure

In this example, double-blinded, randomized, subjects with a history of gout were provided a transdermal formulation of the present disclosure or a placebo lotion. And, the study assayed the effectiveness of the transdermal formulation for early treatment intervention in subjects experiencing inflammatory pain, e.g., from acute gout flare. Also measured was changes in C-Reactive Protein levels.

When a subject later experienced pain from an acute gout flare, they were to follow the study protocol of applying the transdermal formulation of the present disclosure or a placebo lotion to the limb associated with the flaring joint, every 4 hours for 3 applications each day. The study product was packaged in individualized doses of 10 ml lotion. Subjects were instructed to apply one dose to the entire limb of the gout involved target joint (e.g., if a toe was affected, the 10 ml dose would be applied to the entire leg). Subjects in both groups also orally ingested the standard of care drug (Colchicine at 1.2 mg followed by 0.6 mg 1 hour later, according to Colcrys PI). In this study, the study product was a transdermal formulation comprising 33% sodium bicarbonate and 0.5% menthol in transdermal delivery lotion.

At regular intervals, subjects self-reported their symptoms on an eDiary system.

During the treatment phase of the study, subjects were followed for 7 days. The subjects provided a target joint pain score using a 0-10 pain-numeric rating on a daily basis using their eDiary. The subjects took daily blood pressure measurements and entered them into their eDiary. The subjects recorded how many times they applied the study lotion per day. The subject also completed other questionnaires on the eDiary including PROMIS PF 20, and PGART on days 2 and 7, as well as documenting any changes in concomitant medications (including rescue medications) or procedures.

Within 24 hours of beginning the study protocol, the subject returned to the clinic for a non-fasting blood draw which was used, in part, to determine baseline blood or serum C-Reactive Proteins levels, clinician assessment of tenderness and swelling of the target joint, blood pressure measurement, and update of concomitant medications/procedures.

At the end of the follow-up period (7 days), the subject returned to the clinic for a final non-fasting blood draw which was used, in part, to determine baseline blood or serum C-Reactive Proteins levels, clinician assessments of tenderness and swelling of the target joint, blood pressure measurement, and update of concomitant medications/procedures. At this final visit the subject completed a blinding questionnaire, and questionnaires about product use and attributes.

The primary endpoint of the study was to determine if a transdermal formulation of the present disclosure effectively and safely reduced pain associated with an acute gout flare compared to placebo. The primary efficacy endpoint was the Sum of Pain Intensity Difference (SPID) score through day 7. SPID scores were computed by subtracting the baseline pain-numeric rating from each of the subsequent pain scores assessed at 0.25-, 0.5-, 4-, 8-, 24-, 48-, 72-, 96-, 120-, 144-, and 168-hours post-baseline. The final SPID score was the time weighted sum of the individual SPIDs. The primary efficacy analysis compared the treatment arm with the placebo group using an analyses of covariance (ANCOVA) model which included a factor for treatment and baseline pain value as a covariate. Completion of least square means between the treatment arm and the placebo group was also performed. Time to resolution was assessed by greater than or equal to 50% reduction in joint pain score from baseline of the acute gout flare using Kaplan-Meier methods. Subjects who use rescue pain medication, discontinue use of study drug, or otherwise have missing pain score data will be censored at time of last valid pain score prior to start of pain medication or discontinuation of study drug. Change in baseline pain scores using an 11-point pain-numeric rating in the target joint over the 7-day treatment period was analyzed at each time point using the same ANCOVA model described for the primary endpoint.

The secondary endpoints were to determine if time to resolution is shortened when comparing active to placebo, if there was a reduction in rescue medication usage, if there was a positive clinical response at 48 and 72 hours, if there was reduced tenderness of target joint, if there was reduced swelling of target joint, if there was an increase in PROMIS PF-20 scores, and if there was an improvement in physical function. Pain was assessed using an 11-point numeric scale ranging from no pain at 0 to the worst pain imaginable at 10. The Patient-Reported Outcomes Measurement Information System (PROMIS) is an NIH-funded initiative to develop and validate patient reported outcomes (PROs) for clinical research and practice. (See the World Wide Web (at) nia.nih.gov/research/resource/patient-reported-outcomes-measurement-information-system-promis). The PROMIS 20 is a set of person-centered measures that evaluates and monitors physical function. The Patient Global Assessment of Response to Treatment (PGART) is a feasible and valid patient reported measure of improvement that shows within—and between—group discrimination in levels of improvement. This patient global assessment is one of the five core domains endorsed by Outcome Measures in Rheumatology (OMERACT). The 5-point PGART Likert scale is as follows: “How would you rate your response to treatment?”: 0=excellent; 1=very good; 2=good; 3=fair; or 4=poor response to treatment. Swelling was assessed by a physical or qualified clinical using a LIKERT 4-point scale ranging from: 0—no swelling; 1—mild swelling; 2—moderate swelling; 3—severe swelling (or bulging beyond joint margins). Tenderness was assessed by a physical or qualified clinical using a 3-point LIKERT scale ranging from: 0=Patients States “no pain”; 1=Patient States “there is pain when touched”; 2=Patient states “there is pain and winces”; 3=Patient states “there is pain, winces and withdraws”.

Subjects with a diagnosis of gout using ACR/EULAR criteria (Score ≥8), ages 18-75, history of ≥2 gout flares in 12 months preceding randomization, and on stable doses of urate lowering therapy were included. Exclusion criteria were BMI >40 kg/m², >12 gout flares in the year prior to randomization, history of rheumatoid arthritis, psoriatic arthritis, evidence of septic arthritis, acute polyarticular gout (≥4 joints), and arthritis due to any other cause. Subject characteristics in the study are shown below in Table 20:

TABLE 20

| | | Full Analyses Set | | | Per Protocol | | |
|---|---|---|---|---|---|---|---|
| | | [symbol] (N = 48) | Placebo (N = 5 [symbol] ) | Total (N = [symbol] 8) | DVY7 [symbol] 2 (N = 28) | Placebo (N = 2 [symbol] ) | Total (N = 57) |
| Age | Mean | 53.5 | 54 | 53. [symbol] | [symbol] | 53.2 | [symbol] |
| | SD | 10.8 | 10.67 | 1 [symbol] | 10. [symbol] | 11.1 | 10. [symbol] |
| Sex n (%) | Female | 4 (8.3) | 3 (5.0) | 7 ( [symbol] .1) | 3 [symbol] | 2 (6. [symbol] ) | [symbol] |
| | Male | 44 ( [symbol] ) | 47 [symbol] | [symbol] | 2 [symbol] | [symbol] | [symbol] |
| Race n (%) | Asian | 3 (6.3) | [symbol] | 3 [symbol] | 2 (7.1) | 0 | 2 ( [symbol] ) |
| | Black/African | 12 ( [symbol] ) | [symbol] (34.0) | [symbol] | 8 ( [symbol] ) | 11 ( [symbol] ) | [symbol] |
| | White | [symbol] (54. [symbol] ) | 30 ( [symbol] .0) | 51 [symbol] | [symbol] | 17 ( [symbol] ) | [symbol] |
| | [symbol] | 2 (4.2) | 3 ( [symbol] .0) | [symbol] | 1 (3. [symbol] ) | [symbol] | 2(3.5) |
| Height | Mean | 17 [symbol] . [symbol] | 17 [symbol] .7 | [symbol] | 175. [symbol] | 17 [symbol] | 175.3 |
| | SD | [symbol] .31 | 7.71 | [symbol] | 10.41 | 7.82 | 9.1 |
| Weight (kg) | Mean | [symbol] | 95. [symbol] | [symbol] | 1 [symbol] 1.17 | [symbol] 5.4 | [symbol] |
| | SD | 2 [symbol] | 15. [symbol] | 1 [symbol] | 23.2 [symbol] 4 | [symbol] | 2 [symbol] |
| BMI | Mean | 32.82 | 31.04 | 31. [symbol] 1 | 32.5 [symbol] | 31.12 | 31.82 |
| | SD | 5.0 [symbol] 3 | 4. [symbol] 44 | 4. [symbol] | 5.4 [symbol] | 5.385 | [symbol] |
| Baseline NRS Pain Score | Mean | [symbol] | 7.2 | 7.2 | 7.3 | 7.2 | 7.2 |
| | SD | 1.32 | 1. [symbol] | 1.41 | 1.12 | 1.36 | 1.2 [symbol] |

[symbol]

indicates data missing or illegible when filed

In Table 20, the subject that received a transdermal formulation of the present disclosure are identified as DYV702 and as illustrated in Table 17.

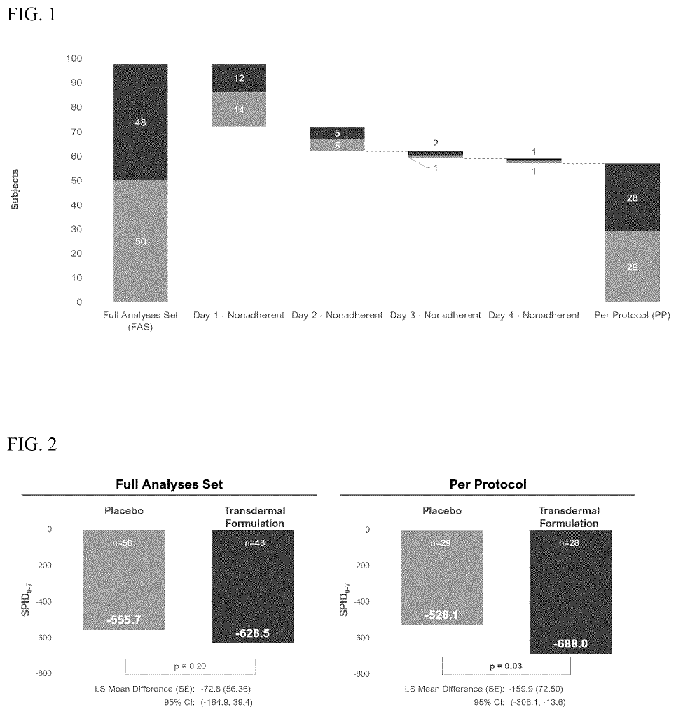

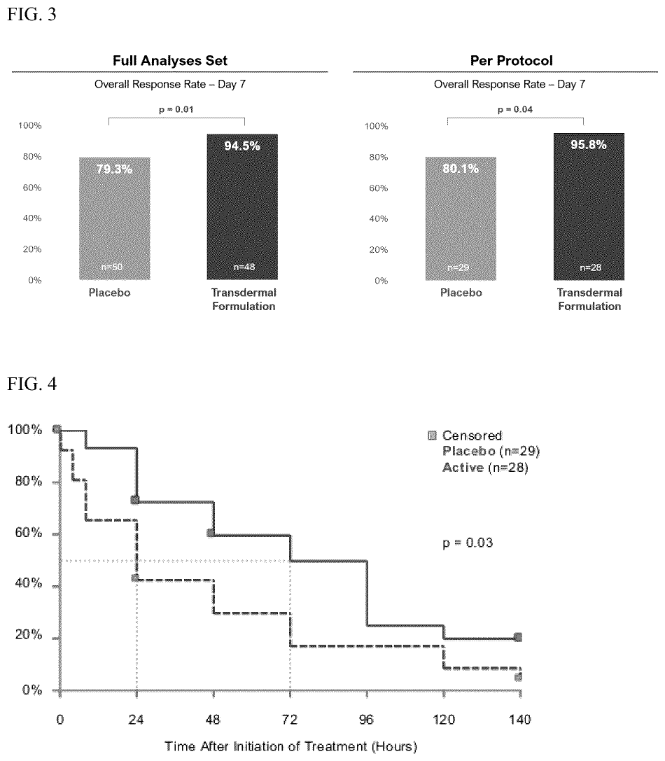

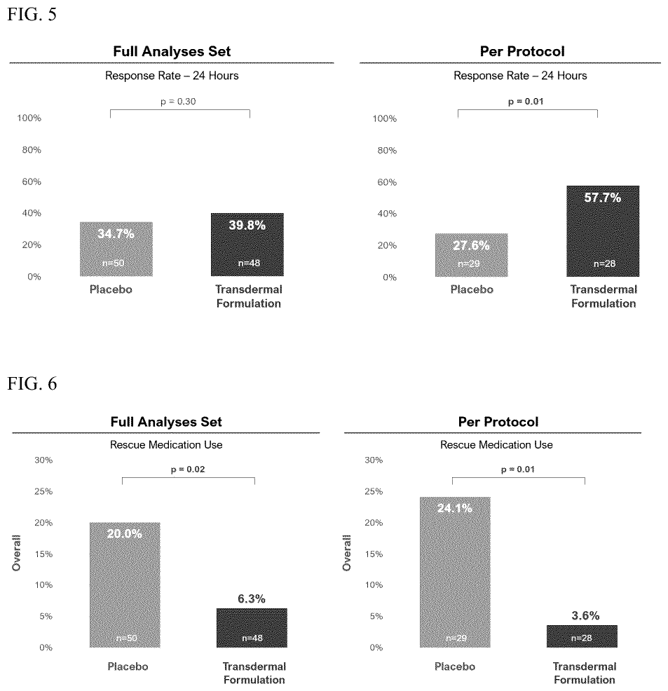

As expected, not all subjects faithfully followed the study protocol. Thus, Table 20 and FIG. 1 (and subsequent figure) distinguish data from those enrolled in the study those in the “Full Analyses Set” and those who followed the study protocol throughout the “Per Protocol” subjects. FIG. 1 is a graph showing a timeline of compliance of subjects in the study described in the Examples. 26 of the 41 non-adherent subjects were non-adherent on day 1. The non-adherence likely led to the differences in results observed between the Full Analysis Set subjects and the Per Protocol subjects as shown in FIG. 2, FIG. 3, and FIG. 5 to FIG. 10.

FIG. 2 includes graphs showing results for the Primary Endpoint—Improved SPIDoa 7 ays for the study subjects who received transdermal formulations of the present disclosure. Data for the Full Analysis Set subjects and the Per Protocol subjects are shown. The transdermal formulations and methods of the present disclosure provided statistically superior pain relief over 7 days compared to placebo in the Per Protocol subject population. Thus, the study met its primary endpoint: ˜30% improvement in SPID₀₋₇ (p=0.03).

FIG. 3 includes graphs showing an improvement in overall responder rates for the study subjects who received transdermal formulations of the present disclosure. The transdermal formulations and methods of the present disclosure provided a statistically significant overall response rate in pain resolution, i.e., an at least a 50% reduction in baseline pain, in 19 out of 20 subjects who received the per protocol transdermal formulation of the present disclosure by day 7 (79% vs 96%, p=0.04).

FIG. 4 includes a graph showing improved time to resolution of pain for the Per Protocol subject population that received transdermal formulations of the present disclosure. Resolution is defined as an at least 50% in baseline pain. Per Protocol subjects showed especially rapid resolution of pain relative to the placebo subjects. More specifically, there was an about three-fold improvement in median time to resolution of about 72 hours with placebo versus 24 hours for the per protocol transdermal formulation of the present disclosure (p=0.03).

FIG. 5 includes graphs showing an improved 24-hour response rate for the study subjects who received transdermal formulations of the present disclosure. Notably, the 24-Hour response rate was twice that for subject in the placebo group (28%) versus the per protocol subjects (58%) who received the prescribed dose during the first 24 hours (p=0.01).

FIG. 6 includes graphs showing a reduction in use of rescue medications for the study subjects who received transdermal formulations of the present disclosure. The transdermal formulations and methods of the present disclosure provided a statistically significantly decrease in rescue medication usage in the subjects who received the per protocol transdermal formulation of the present disclosure (24% vs 4%, p=0.01)

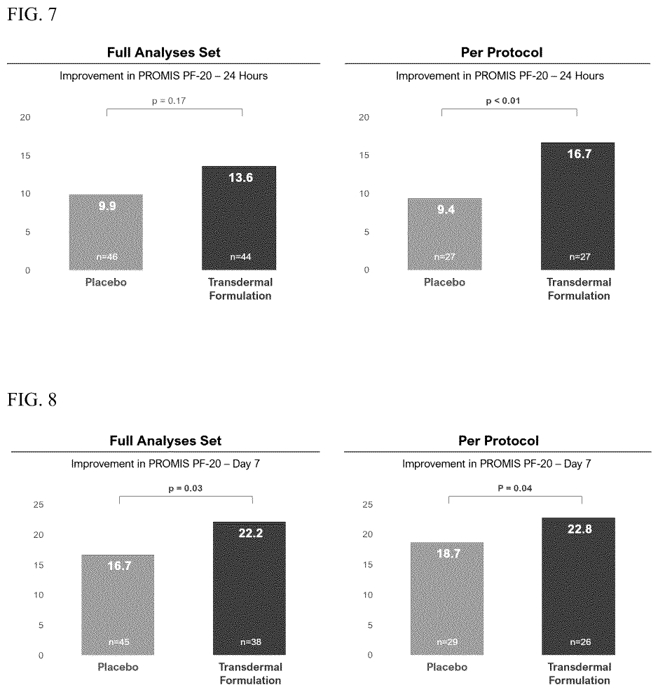

FIG. 7 includes graphs showing an improvement in Patient-Rated Physical Function (PROMIS PF-20) by 24 hours for the study subjects who received transdermal formulations of the present disclosure. The transdermal formulations and methods of the present disclosure provided a statistically significantly improvement in PROMIS PF-20 scores at 24 hours. A change in 5 points is viewed as a clinically relevant change; notably, the average improvement for Per Protocol subjects was 16.7 (p<0.01).

FIG. 8 includes graphs showing an improvement in Patient-Rated Physical Function (PROMIS PF-20) for the study subjects who received transdermal formulations of the present disclosure. The transdermal formulations and methods of the present disclosure provided a statistically significantly improvement in PROMIS PF-20 scores that were sustained through day 7. A change in 5 points is viewed as a clinically relevant change; notably, the average improvement for Per Protocol subjects was 22.8 (p=0.04).

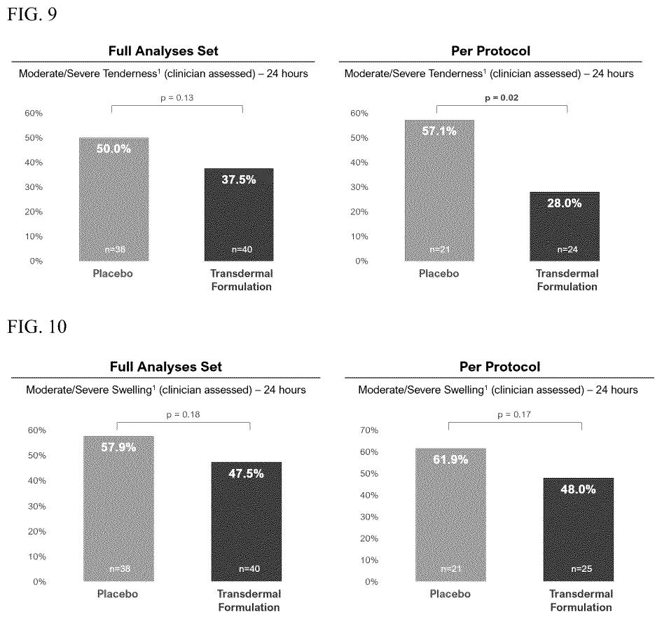

FIG. 9 includes graphs showing reduction in moderate/severe tenderness at 24 hours for the study subjects who received transdermal formulations of the present disclosure. The transdermal formulations and methods of the present disclosure provided a statistically significantly reduction in moderate/severe tenderness. Subjects experienced a 50% reduction in moderate/severe joint tenderness at 24 hours in 57% of placebo subjects versus 28% for subjects who received the per protocol transdermal formulation of the present disclosure (p=0.02) FIG. 10 includes graphs showing reduction in moderate/severe swelling at 24 hours for the study subjects who received transdermal formulations of the present disclosure. The transdermal formulations and methods of the present disclosure provided a non-significantly reduction in moderate/severe swelling.

However, since swelling takes much longer to resolve; a significant reduction in swelling is not expected.

It is noteworthy that the buffering agent in the transdermal formulations of the present disclosure did not exacerbate swelling due to sodium loading.

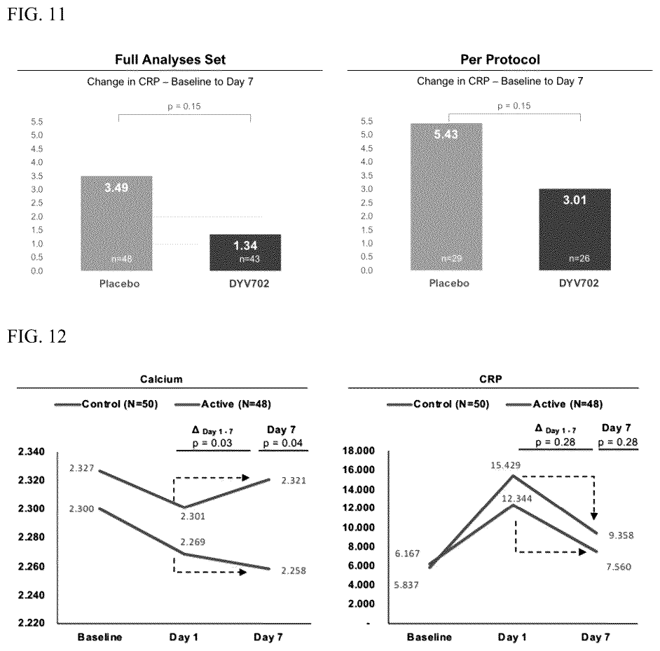

FIG. 11 includes graphs showing changes in C-Reactive Proteins for the study subjects who received transdermal formulations of the present disclosure. As shown, the transdermal formulations and methods of the present disclosure provided a non-significant reduction in C-Reactive Proteins. However, the transdermal formulations of the present disclosure mitigated rise in C-Reactive Proteins by about 50% in both the full analysis set and the per protocol set, each relative to placebo. Had the study subjects been followed for longer than 7 days, it is likely that C-Reactive Protein levels would further lessen in the study subjects who received transdermal formulations of the present disclosure.

FIG. 12 includes a graph showing changes in serum calcium (left image) and a graph showing changes in C-Reactive Proteins (right image) over the seven-day study described in this Example. As shown, subjects in the active group had a statistically significant decrease in serum calcium levels relative to the control populations. This data is particularly interesting as it is known in the art that increases in serum calcium levels are typically derived from bone decalcification. In gout, at least, the decalcification may be related to the body's desire to buffer the increased uric acid resulting from a gout flare. As disclosed herein, the transdermal formulations of the present disclosure provide systemic buffering, which contribute to treating a gout flare. Importantly, this systemic buffering avoids the body's need to decalcify the bones to obtain serum calcium. Without wishing to be bound by theory, the formulations and methods of the present disclosure may prevent bone decalcification due to other pathological conditions, such as osteomalacia and osteoporosis, and in the absence of a gout flare. Thus, the formulations and methods of the present disclosure lower elevated calcium levels in blood or plasma, stabilize calcium levels in blood or plasma to levels prior to a gout flare, reduce symptoms related to osteoporosis, reduce symptoms related to osteomalacia, improve bone density, and/or inhibit and/or reverse bone decalcification. FIG. 12 also shows, in this population of subjects, CRP levels were lower at Day 1 and Day 7 in the treatment arm vs control, however, the difference did not reach significance.

The transdermal formulation of the present disclosure provided an overall higher patient satisfaction at 24 hours. 57% of placebo subjects versus 79% for subjects who received the per protocol transdermal formulation of the present disclosure reported their results as “very good/excellent” (p=0.05).

Additional Data is shown below in Table 20:

TABLE 20
Summary of key clinical efficacy analyses (Intent-to-treat population, N = 98)

| | Control | Active | |
|---|---|---|---|
| | (n = 50) | (n = 48) | p-value |
| Overall Response Rate¹ | 79.3% | 94.5% | 0.01 |
| Rescue Medication Use² | 20.0% | 6.3% | 0.02 |
| Change in PROMIS PF-20 scores, Baseline to Day 7³ | 16.7 | 22.2 | 0.03 |

¹Resolution is defined as at least a 50% reduction in pain; Overall response rate is those reaching resolution by Day 7; Subjects who use rescue pain medication, discontinue use of study drug, or otherwise have missing pain score data were censored; Two-proportion z-test.
²Two-proportion z-test.
³PROMIS-physical function-20 (PF-20) consists of 20 questions, each scored on a 5-point scale (total 0-100), with higher scores indicating better functioning; Analysis of covariance (ANCOVA) with baseline PROMIS PF-20 as a covariate.

Also, there were no safety concerns with no drug-related serious adverse events (SAEs) and subject no dropouts due to an adverse event. The transdermal formulation of the present disclosure surprisingly appeared to have no effect on blood pressure, even though the per protocol of the transdermal formulation included a total of 2520 mg of sodium.

In conclusion, the transdermal formulation of the present disclosure reduced the pain intensity and duration of an acute gout flare with higher overall response rates and faster time to resolution. This led to significant improvements in physical function and patient-reported satisfaction. A notable reduction in rescue medication use and lack of adverse effects makes this topical formulation a promising therapeutic choice; especially during debilitating acute gout flares in patients with concomitant comorbidities.

However, the most surprising result of the study was the reduction in C-Reactive Protein levels in subjects who received the transdermal formulation of the present disclosure. This reduction in C-Reactive Protein levels has implications for treating and prevention the numerous types of inflammation that are associated with C-Reactive Protein, e.g., with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity.

In the present study, the study product was a transdermal formulation comprising 33% of a buffering agent (sodium bicarbonate) and 0.5% menthol in transdermal delivery lotion. However, other transdermal formulations of the present disclosure could be used instead. As examples, the formulation could include a lower amount of a buffering agent, e.g., 10% to 33%, or a higher amount of the buffering agent, e.g., 33% to 50%. The buffering agent may be a different buffer, e.g., sodium carbonate. The transdermal formulation may lack menthol. The transdermal formulation may be formulated as cream or ointment or may impregnate a patch.

In the present study, the subjects received the standard of care (SOC) of oral colchicine. However, colchicine could be combined with the buffering agent in a transdermal formulation or colchicine could be administered in a separate transdermal formulation. Additionally, another medicament could be administered together with a transdermal formulation, e.g., in the transdermal formulation or as a separate composition. As examples, the other medicament may be a nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent (Probenecid) or Krystexxa (pegloticase).

Alternatively, other buffering agents, e.g., Sodium Hydroxide (Sodium oxidanide), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof could have been used in lieu of sodium bicarbonate or sodium carbonate.

Additionally, the formulations and methods of the present disclosure prevent and/or reduce bone decalcification, which least to a relative decrease in serum calcium levels. It is known in the art that increases in serum calcium levels are typically derived from bone decalcification. As disclosed herein, the transdermal formulations of the present disclosure provide systemic buffering, which contribute to treating an inflammation, e.g., inflammatory pain. Importantly, this systemic buffering avoids the body's need to decalcify the bones to obtain serum calcium in the context of inflammation. Without wishing to be bound by theory, the formulations and methods of the present disclosure may prevent bone decalcification due to other pathological conditions, such as osteomalacia and osteoporosis, and in the absence of an inflammation, e.g., inflammatory pain. Thus, the formulations and methods of the present disclosure lower elevated calcium levels in blood or plasma, stabilize calcium levels in blood or plasma to levels prior to inflammation, e.g., inflammatory pain, reduce symptoms related to osteoporosis, reduce symptoms related to osteomalacia, improve bone density, and/or inhibit and/or reverse bone decalcification.

### Example 2: Treatments for Mild to Moderate Pain Due to Inflammation

In this example, treatment methods are described for subjects having mild to moderate pain from inflammation.

A subject having mild to moderate pain associated with inflammation is administered a combination therapy comprising: a transdermal formulation comprising a therapeutically-effective amount of a buffering agent, wherein the buffering agent comprises sodium bicarbonate and/or sodium carbonate; and administering to the subject one of (a) a composition comprising colchicine, (b) a composition comprising a nonsteroidal medicament, or (c) a composition comprising a corticosteroid. In this aspect, mild pain associated with the inflammation being defined as an ACR score of up to 4 and moderate pain associated with the inflammation being defined as an ACR score of 5 or 6, and the combination therapy reduces blood or serum levels of C-Reactive Protein relative to a subject that is not administered the combination therapy. The transdermal formulation may be administered before, contemporaneously with, and/or after (a) the composition comprising colchicine, (b) the composition comprising the nonsteroidal medicament, or (c) the composition comprising the corticosteroid. The subject may be administered two of (a) the composition comprising colchicine, (b) the composition comprising the nonsteroidal medicament, or (c) the composition comprising the corticosteroid. The subject may be administered each of (a) the composition comprising colchicine, (b) the composition comprising the nonsteroidal medicament, or (c) the composition comprising the corticosteroid. The nonsteroidal medicament may be a nonsteroidal anti-inflammatory drug (NSAID), a COX-2 Inhibitor, an Opioid, and/or Illaris (canakinumab) and/or the corticosteroid may be one or more of prednisone, methylprednisolone, prednisolone, triamcinolone, and/or a glucocorticoid. The transdermal formulation may be any herein disclosed transdermal formulation, e.g., as described in any of Table 1 to Table 19 or as described elsewhere herein. In some cases, the transdermal formulation comprises menthol and in other cases, the transdermal formulation lacks menthol.

In some cases, the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

Alternatively, other buffering agents, e.g., Sodium Hydroxide (Sodium oxidanide), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof could have been used in lieu of sodium bicarbonate or sodium carbonate.

### Example 3: Illustrative Methods for Preventing Inflammation

In this example, methods for preventing inflammation, reducing the severity of upcoming inflammation, and/or reducing the likelihood a recurrent inflammation in a subject at risk thereof.

A method preventing inflammation and/or reducing the severity of upcoming inflammation comprises administering to a subject who is not experiencing a symptom of inflammation, a combination therapy comprising: a transdermal formulation comprising a therapeutically-effective amount of a buffering agent, wherein the buffering agent comprises sodium bicarbonate and/or sodium carbonate; and a separate composition comprising a therapeutically-effective amount of colchicine, wherein the colchicine is administered before, contemporaneously with, and/or after administering the transdermal formulation. In this aspect, preventing inflammation and/or reducing the severity of upcoming inflammation is determined in part by a reduction of blood or serum levels of C-Reactive Protein relative to a subject that is not administered the combination therapy.

Additionally or alternately, a method for preventing inflammation and/or reducing the severity of upcoming inflammation comprises administering to a subject who is not experiencing a symptom of inflammation, a transdermal formulation comprising: a therapeutically-effective amount of a buffering agent, wherein the buffering agent comprises sodium bicarbonate and/or sodium carbonate; and a therapeutically-effective amount of colchicine. In this aspect, the transdermal formulation is administered before symptoms inflammation are experienced by the subject and preventing inflammation and/or reducing the severity of upcoming inflammation is determined in part by a reduction of blood or serum levels of C-Reactive Protein relative to a subject that is not administered the transdermal formulation.

Also, a method for reducing the likelihood a recurrent inflammation may comprise administering to a subject that previously has been treated for inflammation, a combination therapy comprising: a transdermal formulation comprising a therapeutically-effective amount of a buffering agent, wherein the buffering agent comprises sodium bicarbonate and/or sodium carbonate; and a separate composition comprising a therapeutically-effective amount of colchicine, wherein the colchicine is administered before, contemporaneously with, and/or after administering the transdermal formulation. In this aspect, the combination therapy reduces blood or serum levels of C-Reactive Protein relative to a subject that is not administered the combination therapy.

Additionally or alternately, a method for reducing the likelihood a recurrent inflammation may comprise administering to a subject that previously has been treated for inflammation, a transdermal formulation comprising: a therapeutically-effective amount of a buffering agent, wherein the buffering agent comprises sodium bicarbonate and/or sodium carbonate; and a therapeutically-effective amount of colchicine. In this aspect, the transdermal formulation reduces blood or serum levels of C-Reactive Protein relative to a subject that is not administered the transdermal formulation.

The transdermal formulation may be as described in any of Table 1 to Table 19 or as described elsewhere herein.

In these methods, the buffering agent may comprise Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

The dosage of the therapeutically-effective amount of the buffering agent may be less than the dosage used to treat the previous inflammatory event and the dosage of the therapeutically-effective amount of colchicine may be less than the dosage used to treat the previous inflammatory event or the therapeutically-effective amount of colchicine may be the same as the dosage used to treat the previous inflammatory event.

The dosage of the therapeutically-effective amount of the buffering agent may be the same as the dosage used to treat the previous inflammatory event and the dosage of the therapeutically-effective amount of colchicine may be less than the dosage used to treat the previous inflammatory event or the therapeutically-effective amount of colchicine may be the same as the dosage used to treat the previous inflammatory event.

Another therapeutically-effective amount of colchicine may be administered orally or topically before, contemporaneously with, and/or after administering the transdermal formulation.

The transdermal formulation may be administered for at least about a week, at least about two weeks, at least about three weeks, at least about a month, at least about two months, at least about three months, at least about four months, at least about five months, at least about six months, at least about seven months, at least about eight months, at least about nine months, at least about ten months, at least about eleven months, or at least about one year.

The transdermal formulation may be any herein disclosed transdermal formulation. In some cases, the transdermal formulation comprises menthol and in other cases, the transdermal formulation lacks menthol.

Additionally, another medicament could be administered together with the transdermal formulation, e.g., in the transdermal formulation or as a separate composition. As examples, transdermal formulation could be administered in combination with a nonsteroidal medicament is a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent (Probenecid) or Krystexxa (pegloticase).

In some cases, the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

### Example 4: Illustrative Methods for Treating Chronic Inflammation

In this example, methods for treating chronic inflammation are described.

A transdermal formulation of the present disclosure may be used in method of treating chronic inflammation (e.g., as described in any of Table 1 to Table 19 or as described elsewhere herein). The method comprises administering to a subject that previously has been treated for inflammation, the transdermal formulation comprising a therapeutically-effective amount of a buffering agent, wherein the buffering agent comprises sodium bicarbonate and/or sodium carbonate and comprising a therapeutically-effective amount of colchicine. Treating chronic inflammation may be determined in part by a reduction of blood or serum levels of C-Reactive Protein relative to a subject that is not administered the combination therapy.

In these methods, the buffering agent may comprise Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

Another therapeutically-effective amount of colchicine may be administered orally or topically before, contemporaneously with, and/or after administering the transdermal formulation.

The transdermal formulation may be administered for at least about a week, at least about two weeks, at least about three weeks, at least about a month, at least about two months, at least about three months, at least about four months, at least about five months, at least about six months, at least about seven months, at least about eight months, at least about nine months, at least about ten months, at least about eleven months, or at least about one year.

The transdermal formulation may be any herein disclosed transdermal formulation. In some cases, the transdermal formulation comprises menthol and in other cases, the transdermal formulation lacks menthol.

Additionally, another medicament could be administered together with the transdermal formulation, e.g., in the transdermal formulation or as a separate composition. As examples, transdermal formulation could be administered in combination with a nonsteroidal medicament is a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent (Probenecid) or Krystexxa (pegloticase).

In some cases, the chronic inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

### Example 5: C-Reactive Protein is Reduced by Transdermal Formulations and Methods of the Present Disclosure

In this example, subjects with a history of inflammation are provided a transdermal formulation of the present disclosure or a placebo lotion. And, the study assays the effectiveness of the transdermal formulation for early treatment intervention in subjects experiencing inflammatory pain, e.g., from acute gout flare. Also measured are changes in C-Reactive Protein levels. The transdermal formulation may be as described in any of Table 1 to Table 19 or as described elsewhere herein.

When a subject later experiences an acute gout flare, they are to follow the study protocol of applying the apply transdermal formulation of the present disclosure or a placebo lotion to the limb associated with the flaring joint, every 4 hours for 3 applications each day. The study product is packaged in individualized doses of 10 ml lotion. Subjects are instructed to apply one dose to the entire limb of the gout involved target joint (e.g., if a toe is affected, the 10 ml dose would be applied to the entire leg). In this study, the study product is a transdermal formulation comprising 33% sodium bicarbonate and 0.5% menthol in transdermal delivery lotion.

At regular intervals, subjects self-report their symptoms on an eDiary system.

During the treatment phase of the study, subjects are followed for 7 days. The subjects are provided a target joint pain score using a 0-10 pain-numeric rating on a daily basis using their eDiary. The subjects take daily blood pressure measurements and enter them into their eDiary. The subjects record how many times they applied the study lotion per day. The subject also complete other questionnaires on the eDiary including PROMIS PF 20, and PGART on days 2 and 7, as well as documenting any changes in concomitant medications (including rescue medications) or procedures.

Within 24 hours of beginning the study protocol, the subject return to the clinic for a non-fasting blood draw, in part, to determine baseline blood or serum C-Reactive Proteins levels, clinician assessment of tenderness and swelling of the target joint, blood pressure measurement, and update of concomitant medications/procedures.

At the end of the follow-up period (7 days), the subject returns to the clinic for a final non-fasting blood draw, in part, to determine baseline blood or serum C-Reactive Proteins levels, clinician assessments of tenderness and swelling of the target joint, blood pressure measurement, and update of concomitant medications/procedures. At this final visit the subject completes a blinding questionnaire, and questionnaires about product use and attributes.

The primary endpoint of the study is to determine if a transdermal formulation of the present disclosure effectively and safely reduces pain associated with an acute gout flare compared to placebo. The primary efficacy endpoint is the Sum of Pain Intensity Difference (SPID) score through day 7. SPID scores are computed by subtracting the baseline pain-numeric rating from each of the subsequent pain scores assessed at 0.25-, 0.5-, 4-, 8-, 24-, 48-, 72-, 96-, 120-, 144-, and 168-hours post-baseline. The final SPID score is the time weighted sum of the individual SPIDs. The primary efficacy analysis compares the treatment arm with the placebo group using an analyses of covariance (ANCOVA) model which includes a factor for treatment and baseline pain value as a covariate. Completion of least square means between the treatment arm and the placebo group is also performed. Time to resolution is assessed by greater than or equal to 50% reduction in joint pain score from baseline of the acute gout flare using Kaplan-Meier methods.

Subjects who use rescue pain medication, discontinue use of study drug, or otherwise have missing pain score data shall be censored at time of last valid pain score prior to start of pain medication or discontinuation of study drug. Change in baseline pain scores using an 11-point pain-numeric rating in the target joint over the 7-day treatment period is analyzed at each time point using the same ANCOVA model described for the primary endpoint.

An important endpoint of the study is a possible reduction in C-Reactive Protein levels in subjects who received the transdermal formulation of the present disclosure.

The secondary endpoints are to determine if time to resolution is shortened when comparing active to placebo, if there is a reduction in rescue medication usage, if there is a positive clinical response at 48 and 72 hours, if there is reduced tenderness of target joint, if there is reduced swelling of target joint, if there is an increase in PROMIS PF-20 scores, and if there is an improvement in physical function. Pain is assessed using an 11-point numeric scale ranging from no pain at 0 to the worst pain imaginable at 10. The Patient-Reported Outcomes Measurement Information System (PROMIS) is an NIH-funded initiative to develop and validate patient reported outcomes (PROs) for clinical research and practice. (See the World Wide Web (at) nia.nih.gov/research/resource/patient-reported-outcomes-measurement-information-system-promis).

The PROMIS 20 is a set of person-centered measures that evaluates and monitors physical function. The Patient Global Assessment of Response to Treatment (PGART) is a feasible and valid patient reported measure of improvement that shows within—and between—group discrimination in levels of improvement.

This patient global assessment is one of the five core domains endorsed by Outcome Measures in Rheumatology (OMERACT). The 5-point PGART Likert scale is as follows: “How would you rate your response to treatment?”: 0=excellent; 1=very good; 2=good; 3=fair; or 4=poor response to treatment. Swelling is assessed by a physical or qualified clinical using a LIKERT 4-point scale ranging from: 0—no swelling; 1—mild swelling; 2—moderate swelling; 3—severe swelling (or bulging beyond joint margins). Tenderness is assessed by a physical or qualified clinical using a 3-point LIKERT scale ranging from: 0=Patients States “no pain”; 1=Patient States “there is pain when touched”; 2=Patient states “there is pain and winces”; 3=Patient states “there is pain, winces and withdraws”.

Subjects with a diagnosis of gout using ACR/EULAR criteria (Score ≥8), ages 18-75, history of ≥2 gout flares in 12 months preceding randomization, and on stable doses of urate lowering therapy are included. Exclusion criteria are BMI >40 kg/m², >12 gout flares in the year prior to randomization, history of rheumatoid arthritis, psoriatic arthritis, evidence of septic arthritis, acute polyarticular gout (≥4 joints), and arthritis due to any other cause.

In the present study, the study product is a transdermal formulation comprising 33% of a buffering agent (sodium bicarbonate) and 0.5% menthol in transdermal delivery lotion. However, other transdermal formulations of the present disclosure could be used instead. As examples, the formulation could include a lower amount of a buffering agent, e.g., 10% to 33%, or a lower amount of the buffering agent, e.g., 33% to 50%. The buffering agent may be a different buffer, e.g., sodium carbonate. The transdermal formulation may lack menthol. The transdermal formulation may be formulated as cream or ointment or may impregnate a patch.

Additional medicaments could be administered together with a transdermal formulation, e.g., in the transdermal formulation or as a separate composition. As examples, the additional medicament may be a nonsteroidal medicament, such as a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent (Probenecid) or Krystexxa (pegloticase).

Importantly, the transdermal formulation may be administered before, after, or contemporaneously with a different additional medicament.

Additionally, the formulations and methods of the present disclosure prevent and/or reduce bone decalcification, which least to a relative decrease in serum calcium levels. It is known in the art that increases in serum calcium levels are typically derived from bone decalcification. As disclosed herein, the transdermal formulations of the present disclosure provide systemic buffering, which contribute to treating an inflammation, e.g., inflammatory pain. Importantly, this systemic buffering avoids the body's need to decalcify the bones to obtain serum calcium in the context of inflammation. Without wishing to be bound by theory, the formulations and methods of the present disclosure may prevent bone decalcification due to other pathological conditions, such as osteomalacia and osteoporosis, and in the absence of an inflammation, e.g., inflammatory pain. Thus, the formulations and methods of the present disclosure lower elevated calcium levels in blood or plasma, stabilize calcium levels in blood or plasma to levels prior to inflammation, e.g., inflammatory pain, reduce symptoms related to osteoporosis, reduce symptoms related to osteomalacia, improve bone density, and/or inhibit and/or reverse bone decalcification.

In some cases, the subject has kidney impairment, e.g., the subject has diabetes, chronic kidney disease (CKD), Polycystic kidney disease (PKD), Lupus nephritis, kidney cancer, Alport syndrome, amyloidosis, Goodpasture syndrome, and Wegener's granulomatosis, and/or is a recipient of a renal transplant. Since the formulations, compositions, and/or methods of the present disclosure do not comprise colchicine, they may be used in subjects with kidney impairment. And, this population of gout patients is especially benefited by and treated with the formulations, compositions, and/or methods of the present disclosure.

A reduction in C-Reactive Protein levels in subjects who received the transdermal formulation of the present disclosure has implications for treating and prevention the numerous types of inflammation that are associated with C-Reactive Protein, e.g., with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity.

### Example 6: Illustrative Methods for Preventing Inflammation

In this example, methods for preventing inflammation, reducing the severity of upcoming inflammation, and/or reducing the likelihood a recurrent inflammation in a subject at risk thereof. The transdermal formulation may be as described in any of Table 1 to Table 19 or as described elsewhere herein.

A method for preventing inflammation and/or reducing the severity of upcoming inflammation may comprise administering to a subject who is not experiencing a symptom of inflammation a transdermal formulation comprising a therapeutically-effective amount of a buffering agent, wherein the buffering agent comprises sodium bicarbonate and/or sodium carbonate. The transdermal formulation may reduce blood or serum levels of C-Reactive Protein relative to a subject that is not administered the combination therapy.

Additionally, a method for preventing inflammation and/or reducing the severity of upcoming inflammation may comprise administering to a subject who is not experiencing a symptom of inflammation, a combination therapy comprising: a transdermal formulation comprising a therapeutically-effective amount of a buffering agent, wherein the buffering agent comprises sodium bicarbonate and/or sodium carbonate; and a separate composition comprising a therapeutically-effective amount of a nonsteroidal medicament and/or a corticosteroid. The separate composition may be administered before, contemporaneously with, and/or after administering the transdermal formulation; and the combination therapy reduces blood or serum levels of C-Reactive Protein relative to a subject that is not administered the combination therapy. In some case, the separate composition is administered orally or topically before, contemporaneously with, and/or after administering the transdermal formulation. The nonsteroidal medicament may be a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

Also, a method for preventing inflammation and/or reducing the severity of upcoming inflammation may comprise administering to a subject who is not experiencing a symptom of inflammation, a transdermal formulation comprising: a therapeutically-effective amount of a buffering agent, wherein the buffering agent comprises sodium bicarbonate and/or sodium carbonate; and a therapeutically-effective amount of a nonsteroidal medicament and/or a corticosteroid. The transdermal formulation may be administered before symptoms inflammation are experienced by the subject and preventing inflammation and/or reducing the severity of upcoming inflammation is determined in part by a reduction of blood or serum levels of C-Reactive Protein relative to a subject that is not administered the combination therapy. The nonsteroidal medicament may be a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

In a method for reducing the likelihood a recurrent inflammation, the method may comprise administering to a subject that previously has been treated for inflammation, a combination therapy comprising administering to the subject a transdermal formulation comprising a therapeutically-effective amount of a buffering agent, wherein the buffering agent comprises sodium bicarbonate and/or sodium carbonate. The transdermal formulation may reduce blood or serum levels of C-Reactive Protein relative to a subject that is not administered the combination therapy. The dosage of the therapeutically-effective amount of the buffering agent may be less than or the same as the dosage used to previously treat inflammation.

Additionally, a method for reducing the likelihood a recurrent inflammation may comprise administering to a subject that previously has been treated for inflammation, a transdermal formulation comprising: a therapeutically-effective amount of a buffering agent, wherein the buffering agent comprises sodium bicarbonate and/or sodium carbonate; and a therapeutically-effective amount of a nonsteroidal medicament and/or a corticosteroid. The transdermal formulation may reduce blood or serum levels of C-Reactive Protein relative to a subject that is not administered the combination therapy. In some cases, the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event. The dosage of the therapeutically-effective amount of the buffering agent may be the same as the dosage used to treat the previous inflammatory event and the dosage of the therapeutically-effective amount of the nonsteroidal medicament and/or the corticosteroid may be less than the dosage used to treat the previous inflammatory event or the therapeutically-effective amount of the nonsteroidal medicament and/or the corticosteroid may be the same as the dosage used to treat the previous inflammatory event. The other therapeutically-effective amount of the nonsteroidal medicament and/or the corticosteroid may be administered orally or topically before, contemporaneously with, and/or after administering the transdermal formulation. The nonsteroidal medicament may be a nonsteroidal anti-inflammatory drug (NSAID), a COX-2 Inhibitor, an Opioid, and/or Illaris (canakinumab) and/or the corticosteroid may be one or more of prednisone, methylprednisolone, prednisolone, triamcinolone, and/or a glucocorticoid.

Also, in a method for reducing the likelihood a recurrent inflammation, the method may comprise administering to a subject that previously has been treated for inflammation, administering to the subject a combination therapy comprising: a transdermal formulation comprising a therapeutically-effective amount of a buffering agent, wherein the buffering agent comprises sodium bicarbonate and/or sodium carbonate; and a separate composition comprising a therapeutically-effective amount of a nonsteroidal medicament and/or a corticosteroid. The separate composition may be administered before, contemporaneously with, and/or after administering the transdermal formulation; and the transdermal formulation reduces blood or serum levels of C-Reactive Protein relative to a subject that is not administered the combination therapy.

In the above methods, transdermal formulation is administered for at least about a week, at least about two weeks, at least about three weeks, at least about a month, at least about two months, at least about three months, at least about four months, at least about five months, at least about six months, at least about seven months, at least about eight months, at least about nine months, at least about ten months, at least about eleven months, or at least about one year.

The transdermal formulation may be any herein disclosed transdermal formulation. In some cases, the transdermal formulation comprises menthol and in other cases, the transdermal formulation lacks menthol.

Additionally, another medicament could be administered together with the transdermal formulation, e.g., in the transdermal formulation or as a separate composition. As examples, transdermal formulation could be administered in combination with a nonsteroidal medicament is a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent (Probenecid) or Krystexxa (pegloticase).

In some cases, the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

### Example 7: Illustrative Methods for Treating Chronic Inflammation

In this example, methods for treating chronic inflammation are described. The transdermal formulation may be as described in any of Table 1 to Table 19 or as described elsewhere herein.

A method for treating chronic inflammation in a subject that previously has been treated for inflammation, may comprises administering a transdermal formulation comprising a therapeutically-effective amount of a buffering agent, wherein the buffering agent comprises sodium bicarbonate and/or sodium carbonate.

The transdermal formulation may reduce blood or serum levels of C-Reactive Protein relative to a subject that is not administered the combination therapy. The dosage of the therapeutically-effective amount of the buffering agent may be the same or may be less than the dosage used to previously treat inflammation.

Additionally, a method for treating chronic inflammation may comprise administering to a subject that previously has been treated for inflammation, a transdermal formulation comprising: a therapeutically-effective amount of a buffering agent, wherein the buffering agent comprises sodium bicarbonate and/or sodium carbonate. The therapeutically-effective amount of a nonsteroidal medicament and/or a corticosteroid and the transdermal formulation may reduce blood or serum levels of C-Reactive Protein relative to a subject that is not administered the combination therapy. The dosage of the therapeutically-effective amount of the buffering agent may be less than the dosage used to treat the previous inflammatory event and the dosage of the therapeutically-effective amount of the nonsteroidal medicament and/or the corticosteroid may be less than the dosage used to treat the previous inflammatory event or the therapeutically-effective amount of the nonsteroidal medicament and/or the corticosteroid may be the same as the dosage used to treat the previous inflammatory event. The dosage of the therapeutically-effective amount of the buffering agent may be the same as the dosage used to treat the previous inflammatory event and the dosage of the therapeutically-effective amount of the nonsteroidal medicament and/or the corticosteroid may be less than the dosage used to treat the previous inflammatory event or the therapeutically-effective amount of the nonsteroidal medicament and/or the corticosteroid may be the same as the dosage used to treat the previous inflammatory event. In some cases, another therapeutically-effective amount of the nonsteroidal medicament and/or the corticosteroid is administered orally or topically before, contemporaneously with, and/or after administering the transdermal formulation.

In a method for treating chronic inflammation, a subject that previously has been treated for inflammation, a combination therapy is administered a transdermal formulation comprising a therapeutically-effective amount of a buffering agent, wherein the buffering agent comprises sodium bicarbonate and/or sodium carbonate; and a separate composition comprising a therapeutically-effective amount of a nonsteroidal medicament and/or a corticosteroid. The separate composition may be administered before, contemporaneously with, and/or after administering the transdermal formulation; and the combination therapy reduces blood or serum levels of C-Reactive Protein relative to a subject that is not administered the combination therapy. The separate composition may be administered orally or topically before, contemporaneously with, and/or after administering the transdermal formulation. The dosage of the therapeutically-effective amount of the buffering agent may be less than the dosage used to treat the previous inflammatory event and the dosage of the therapeutically-effective amount of the nonsteroidal medicament and/or the corticosteroid may be less than the dosage used to treat the previous inflammatory event or the therapeutically-effective amount of the nonsteroidal medicament and/or the corticosteroid may be the same as the dosage used to treat the previous inflammatory event. In some cases, the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

The transdermal formulation may be administered for at least about a week, at least about two weeks, at least about three weeks, at least about a month, at least about two months, at least about three months, at least about four months, at least about five months, at least about six months, at least about seven months, at least about eight months, at least about nine months, at least about ten months, at least about eleven months, or at least about one year.

The transdermal formulation may be any herein disclosed transdermal formulation. In some cases, the transdermal formulation comprises menthol and in other cases, the transdermal formulation lacks menthol.

Additionally, another medicament could be administered together with the transdermal formulation, e.g., in the transdermal formulation or as a separate composition. As examples, transdermal formulation could be administered in combination with a nonsteroidal medicament is a nonsteroidal anti-inflammatory drug (NSAID), a COX-2 Inhibitor, an Opioid Illaris (canakinumab), a Xanthine Oxidase Inhibitor (Allopurinol, febuxostat), and/or a Uricosuric agent (Probenecid) or Krystexxa (pegloticase).

In some cases, the chronic inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

In some cases, the subject has kidney impairment, e.g., the subject has diabetes, chronic kidney disease (CKD), Polycystic kidney disease (PKD), Lupus nephritis, kidney cancer, Alport syndrome, amyloidosis, Goodpasture syndrome, and Wegener's granulomatosis, and/or is a recipient of a renal transplant. Since the formulations, compositions, and/or methods of the present disclosure do not comprise colchicine, they may be used in subjects with kidney impairment. And, this population of gout patients is especially benefited by and treated with the formulations, compositions, and/or methods of the present disclosure.

What is claimed is:

1. A method for treating or reducing inflammation in a subject in need thereof, the method comprising administering to the subject a transdermal formulation comprising a therapeutically-effective amount of a buffering agent; and
wherein the transdermal formulation reduces blood or serum levels of C-Reactive Protein relative to a subject that is not administered the combination therapy.

2. The method of claim 1, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

3. The method of claim 1 or claim 2, wherein administering the transdermal formulation further
reduces or eliminates the need for a rescue medicine,
improves the subject's physical function as measured by a Patient-Reported Outcomes Measurement Information System (PROMIS) score, e.g., PROMIS PF 20,
provides an improvement in the subject's Sum of Pain Intensity Difference (SPID) score,
lowers the subject's pain-numeric rating,
decreases the time to resolution of pain relative to a historical control patient,
lowers the subject-reported or physician-assessed moderate-to-severe joint tenderness,
lowers the subject-reported or physician-assessed moderate-to-severe joint swelling,
reduces uric acid crystal levels in blood or plasma,
raises urine pH,
lowers elevated calcium levels in blood or plasma,
stabilizes calcium levels in blood or plasma to levels prior to an inflammatory event,
reduces symptoms related to osteoporosis,
reduces symptoms related to osteomalacia,
improves bone density,
inhibits and/or reverses bone decalcification, and/or
increases patient satisfaction.

4. A method for treating or reducing inflammation in a subject in need thereof, the method comprising administering to the subject a combination therapy comprising:
a transdermal formulation comprising a therapeutically-effective amount of a buffering agent; and
a separate composition comprising a therapeutically-effective amount of a nonsteroidal medicament and/or a corticosteroid,
wherein the separate composition is administered before, contemporaneously with, and/or after administering the transdermal formulation; and
wherein the combination therapy reduces blood or serum levels of C-Reactive Protein relative to a subject that is not administered the combination therapy.

5. The method of claim 4, wherein the separate composition is administered orally or topically before, contemporaneously with, and/or after administering the transdermal formulation.

6. The method of claim 4 or claim 5, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

7. The method of any one of claims 4 to 6, wherein administering the combination therapy further
reduces or eliminates the need for a rescue medicine,
improves the subject's physical function as measured by a Patient-Reported Outcomes Measurement Information System (PROMIS) score, e.g., PROMIS PF 20,
provides an improvement in the subject's Sum of Pain Intensity Difference (SPID) score,
lowers the subject's pain-numeric rating,
decreases the time to resolution of pain relative to a historical control patient,
lowers the subject-reported or physician-assessed moderate-to-severe joint tenderness,
lowers the subject-reported or physician-assessed moderate-to-severe joint swelling,
reduces uric acid crystal levels in blood or plasma,
raises urine pH,
lowers elevated calcium levels in blood or plasma,
stabilizes calcium levels in blood or plasma to levels prior to an inflammatory event,
reduces symptoms related to osteoporosis,
reduces symptoms related to osteomalacia,
improves bone density,
inhibits and/or reverses bone decalcification, and/or
increases patient satisfaction.

8. The method of any one of claims 4 to 7, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

9. A method for treating or reducing inflammation in a subject in need thereof, the method comprising administering to the subject a transdermal formulation comprising:
a therapeutically-effective amount of a buffering agent; and
a therapeutically-effective amount of a nonsteroidal medicament and/or a corticosteroid,
wherein the transdermal formulation reduces blood or serum levels of C-Reactive Protein relative to a subject that is not administered the combination therapy.

10. The method of claim 9, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

11. The method of claim 9 or claim 10, wherein administering the transdermal formulation further
reduces or eliminates the need for a rescue medicine,
improves the subject's physical function as measured by a Patient-Reported Outcomes Measurement Information System (PROMIS) score, e.g., PROMIS PF 20,
provides an improvement in the subject's Sum of Pain Intensity Difference (SPID) score,
lowers the subject's pain-numeric rating,
decreases the time to resolution of pain relative to a historical control patient,
lowers the subject-reported or physician-assessed moderate-to-severe joint tenderness,
lowers the subject-reported or physician-assessed moderate-to-severe joint swelling,
reduces uric acid crystal levels in blood or plasma,
raises urine pH,
lowers elevated calcium levels in blood or plasma,
stabilizes calcium levels in blood or plasma to levels prior to an inflammatory event,
reduces symptoms related to osteoporosis,
reduces symptoms related to osteomalacia,
improves bone density,
inhibits and/or reverses bone decalcification, and/or
increases patient satisfaction.

12. The method of any one of claims 9 to 11, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

13. A method for preventing inflammation and/or reducing the severity of upcoming inflammation, the method comprising administering to a subject who is not experiencing a symptom of inflammation a transdermal formulation comprising a therapeutically-effective amount of a buffering agent; and
wherein the transdermal formulation reduces blood or serum levels of C-Reactive Protein relative to a subject that is not administered the combination therapy.

14. The method of claim 13, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

15. A method for preventing inflammation and/or reducing the severity of upcoming inflammation, the method comprising administering to a subject who is not experiencing a symptom of inflammation, a combination therapy comprising:
a transdermal formulation comprising a therapeutically-effective amount of a buffering agent; and
a separate composition comprising a therapeutically-effective amount of a nonsteroidal medicament and/or a corticosteroid,
wherein the separate composition is administered before, contemporaneously with, and/or after administering the transdermal formulation; and
wherein the combination therapy reduces blood or serum levels of C-Reactive Protein relative to a subject that is not administered the combination therapy.

16. The method of claim 15, wherein the separate composition is administered orally or topically before, contemporaneously with, and/or after administering the transdermal formulation.

17. The method of claim 15 or claim 16, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

18. The method of any one of claims 15 to 17, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

19. A method for preventing inflammation and/or reducing the severity of upcoming inflammation, the method comprising administering to a subject who is not experiencing a symptom of inflammation, a transdermal formulation comprising:
a therapeutically-effective amount of a buffering agent; and
a therapeutically-effective amount of a nonsteroidal medicament and/or a corticosteroid,
wherein the transdermal formulation is administered before symptoms inflammation are experienced by the subject and
wherein preventing inflammation and/or reducing the severity of upcoming inflammation is determined in part by a reduction of blood or serum levels of C-Reactive Protein relative to a subject that is not administered the combination therapy.

20. The method of claim 19, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

21. The method of claim 19 or claim 20, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

22. A method for reducing the likelihood a recurrent inflammation, the method comprising administering to a subject that previously has been treated for inflammation, a transdermal formulation comprising a therapeutically-effective amount of a buffering agent; and
wherein the transdermal formulation reduces blood or serum levels of C-Reactive Protein relative to a subject that is not administered the combination therapy.

23. The method of claim 22, wherein the dosage of the therapeutically-effective amount of the buffering agent is less than the dosage used to previously treat inflammation.

24. The method of claim 22, wherein the dosage of the therapeutically-effective amount of the buffering agent is the same as the dosage used to previously treat inflammation.

25. The method of any one of claims 22 to 24, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

26. A method for reducing the likelihood a recurrent inflammation, the method comprising administering to a subject that previously has been treated for inflammation, a transdermal formulation comprising:
a therapeutically-effective amount of a buffering agent; and
a therapeutically-effective amount of a nonsteroidal medicament and/or a corticosteroid,
wherein the transdermal formulation reduces blood or serum levels of C-Reactive Protein relative to a subject that is not administered the combination therapy.

27. The method of claim 26, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

28. The method of claim 26, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

29. The method of any one of claims 26 to 28, wherein another therapeutically-effective amount of the nonsteroidal medicament and/or the corticosteroid is administered orally or topically before, contemporaneously with, and/or after administering the transdermal formulation.

30. The method of any one of claims 26 to 29, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

31. The method of any one of claims 26 to 30, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

32. A method for reducing the likelihood a recurrent inflammation, the method comprising administering to a subject that previously has been treated for inflammation, administering to the subject a combination therapy comprising:
a transdermal formulation comprising a therapeutically-effective amount of a buffering agent; and
a separate composition comprising a therapeutically-effective amount of a nonsteroidal medicament and/or a corticosteroid,
wherein the separate composition is administered before, contemporaneously with, and/or after administering the transdermal formulation; and
wherein the transdermal formulation reduces blood or serum levels of C-Reactive Protein relative to a subject that is not administered the combination therapy.

33. The method of claim 32, wherein the separate composition is administered orally or topically before, contemporaneously with, and/or after administering the transdermal formulation.

34. The method of claim 32 or claim 33, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

35. The method of claim 32 or claim 33, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

36. The method of any one of claims 32 to 35, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

37. The method of any one of claims 32 to 36, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

38. A method for treating chronic inflammation, the method comprising administering to a subject that previously has been treated for inflammation, a transdermal formulation comprising a therapeutically-effective amount of a buffering agent; and
wherein the transdermal formulation reduces blood or serum levels of C-Reactive Protein relative to a subject that is not administered the combination therapy.

39. The method of claim 38, wherein the dosage of the therapeutically-effective amount of the buffering agent is less than the dosage used to previously treat inflammation.

40. The method of claim 38, wherein the dosage of the therapeutically-effective amount of the buffering agent is the same as the dosage used to previously treat inflammation.

41. The method of any one of claims 38 to 40, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

42. A method for treating chronic inflammation, the method comprising administering to a subject that previously has been treated for inflammation, a transdermal formulation comprising:
a therapeutically-effective amount of a buffering agent; and
a therapeutically-effective amount of a nonsteroidal medicament and/or a corticosteroid,
wherein the transdermal formulation reduces blood or serum levels of C-Reactive Protein relative to a subject that is not administered the combination therapy.

43. The method of claim 42, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

44. The method of claim 42, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

45. The method of any one of claims 42 to 44, wherein another therapeutically-effective amount of the nonsteroidal medicament and/or the corticosteroid is administered orally or topically before, contemporaneously with, and/or after administering the transdermal formulation.

46. The method of any one of claims 42 to 45, wherein the transdermal formulation is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

47. The method of any one of claims 42 to 46, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

48. A method for treating chronic inflammation, the method comprising administering to a subject that previously has been treated for inflammation, a combination therapy comprising:
a transdermal formulation comprising a therapeutically-effective amount of a buffering agent; and
a separate composition comprising a therapeutically-effective amount of a nonsteroidal medicament and/or a corticosteroid,
wherein the separate composition is administered before, contemporaneously with, and/or after administering the transdermal formulation; and
wherein the combination therapy reduces blood or serum levels of C-Reactive Protein relative to a subject that is not administered the combination therapy.

49. The method of claim 48, wherein the separate composition is administered orally or topically before, contemporaneously with, and/or after administering the transdermal formulation.

50. The method of claim 48 or claim 49, wherein the dosage of the therapeutically-effective amount of the buffering agent is less than the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

51. The method of claim 48 or claim 49, wherein the dosage of the therapeutically-effective amount of the buffering agent is the same as the dosage used to treat the previous inflammatory event and the dosage of the therapeutically-effective amount of the nonsteroidal medicament and/or the corticosteroid is less than the dosage used to treat the previous inflammatory event or the therapeutically-effective amount of the nonsteroidal medicament and/or the corticosteroid is the same as the dosage used to treat the previous inflammatory event.

52. The method of any one of claims 48 to 51, wherein the transdermal formulation or the combination therapy is administered for at least about a week, at least about two weeks, at least about three weeks, at least about a month, at least about two months, at least about three months, at least about four months, at least about five months, at least about five months, at least about six months, at least about seven months, at least about eight months, at least about nine months, at least about ten months, at least about eleven months, or at least about one year.

53. The method of any one of claims 48 to 52, wherein the nonsteroidal medicament is a nonsteroidal anti-inflammatory drug (NSAID), a COX-2 Inhibitor, an Opioid, and/or Illaris (canakinumab) and/or wherein the corticosteroid is one or more of prednisone, methylprednisolone, prednisolone, triamcinolone, and/or a glucocorticoid.

54. A method for treating a bone density disorder in a subject in need thereof, the method comprising administering to the subject a transdermal formulation comprising a therapeutically-effective amount of a buffering agent, wherein the transdermal formulation:
lowers elevated calcium levels in blood or plasma,
stabilizes calcium levels in blood or plasma to levels prior to an inflammatory event,
reduces symptoms related to osteoporosis,
reduces symptoms related to osteomalacia,
improves bone density, and/or
inhibits and/or reverses bone decalcification.

55. The method of any one of claims 1 to 54, wherein the transdermal formulation comprises a penetrant or penetration enhancer comprising one or more of phosphatidyl choline (e.g., Phospholipon® 90 G), isopropyl palmitate (IPP), steric acid, benzyl alcohol, safflower oil, almond oil, oleic acid, polyglyceryl-4 laurate, poloxamer 407, poloxamer 188, poloxamer 124, menthol, propylene glycol, cetyl alcohol, isododecane, isopropyl stearate, isopropyl myristate, undecane, xanthan gum, sclerotium gum, pullulan, and lecithin, wherein the lecithin is selected from an egg lecithin, a soy lecithin, and a synthetic lecithin.

56. The method of any one of claims 1 to 55, wherein the transdermal formulation comprises propylene glycol.

57. The method of any one of claims 1 to 56, wherein the transdermal formulation comprises a phospholipid in an amount from about 5% to about 15% w/w of the transdermal formulation; a emollient/moisturizer in an amount from about 10% to about 20% w/w of the transdermal formulation; a fatty acid in an amount from about 0.5% to about 2% w/w of the transdermal formulation; an alcohol in an amount from about 0.5% to about 2% w/w of the transdermal formulation; an oil in an amount from about 1% to about 5% w/w of the transdermal formulation; a surfactant in an amount from about 0.5% to about 2% w/w of the transdermal formulation; the buffering agent in an amount from about 10% to about 50% w/w of the transdermal formulation; and deionized water in an amount to complete the transdermal formulation.

58. The method of any one of claims 1 to 57, wherein the transdermal formulation comprises phosphatidylcholine (e.g., Phospholipon 90 g) in an amount of about 4.03% w/w of the transdermal formulation; benzyl alcohol in an amount of about 1.68% w/w of the transdermal formulation; isopropyl palmitate in an amount of about 7.00% w/w of the transdermal formulation; stearic acid in an amount of about 0.32% w/w of the transdermal formulation; cetyl alcohol in an amount of about 2.00% w/w of the transdermal formulation; menthol in an amount of about 0.50% w/w of the transdermal formulation; ethanol in an amount of about 1.50% w/w of the transdermal formulation; safflower oil in an amount of about 1.55% w/w of the transdermal formulation; oleic acid in an amount of about 0.50% w/w of the transdermal formulation; almond oil in an amount of about 3.00% w/w of the transdermal formulation; propylene glycol in an amount of about 5.00% w/w of the transdermal formulation; dextrose (anhydrous) in an amount of about 0.35% w/w of the transdermal formulation; poloxamer 407 in an amount of about 5.40% w/w of the transdermal formulation; polyglyceryl-4 laurate in an amount of about 1.00% w/w of the transdermal formulation; and a buffering agent in an amount from about 30% to about 35% w/w of the transdermal formulation; and deionized water in an amount to complete the transdermal formulation.

59. The method of claim 57 or claim 58, wherein when the nonsteroidal medicament and/or the corticosteroid is included in the transdermal formulation, the amount of deionized water is reduced to provide for the addition of the therapeutically-effective amount of the nonsteroidal medicament and/or the corticosteroid.

60. The method of any one of claims 1 to 59, wherein the concentration of the buffering agent is from about 10% to about 50% w/w of the transdermal formulation.

61. The method of any one of claims 1 to 60, wherein the buffering agent is Sodium Hydroxide (Sodium oxidanide), Sodium Bicarbonate (baking soda or Sodium hydrogen carbonate), Potassium Bicarbonate (potassium hydrogen carbonate or potassium acid carbonate), Lysine, Tris (Tromethamine, trisaminomethane, 2-amino-2-hydroxymethyl-propane-1,3-diol, or tris(hydroxymethyl)aminomethane), Calcium Carbonate, Sodium Carbonate (Disodium carbonate), Potassium Carbonate, Dipotassium Phosphate (Potassium phosphate dibasic or Potassium hydrogen phosphate), Disodium Phosphate (Sodium phosphate dibasic or Disodium hydrogen phosphate), Trisodium Phosphate, Meglumine ((2R,3R,4R,5S)-6-(Methylamino)hexane-1,2,3,4,5-pentol or Methylglucamine), Arginine, Triethanolamine (TEA or 2,2′,2″-Nitrilotriethanol), Glycine, Monosodium Phosphate (Sodium dihydrogen phosphate), Monopotassium Phosphate (Potassium dihydrogen phosphate), Tripotassium Phosphate (potassium phosphate), Monoethanolamine, Diethanolamine (Diolamine or 2-(2-hydroxyethylamino)ethanol), Magnesium carbonate, 2-imidazole-1-yl-3-ethoxycarbonylpropionic acid (IEPA), or combination thereof.

62. The method of any one of claims 1 to 61, wherein the sodium bicarbonate or sodium carbonate is at a concentration from about 30% to about 35% w/w of the transdermal formulation, e.g., the sodium bicarbonate or sodium carbonate is at a concentration of about 33% w/w of the transdermal formulation.

63. The method of any one of claims 1 to 62, wherein the transdermal formulation comprises menthol.

64. The method of claim 63, wherein the menthol is at a concentration from about 0.1% to about 5.0% w/w of the transdermal formulation.

65. The method of any one of claims 1 to 64, wherein the transdermal formulation comprises about 33% w/w sodium bicarbonate or sodium carbonate and about 0.5% w/w menthol.

66. The method of any one of claims 1 to 65, wherein the transdermal formulation has a pH from about 9 to about 11 or from about 7 to about 10.5.

67. The method of any one of claims 1 to 66, wherein method comprises administering the transdermal formulation about three times a day.

68. The method of any one of claims 1 to 67, wherein the transdermal formulation is formulated as a cream, lotion, or ointment.

69. The method of any of one of claims 1 to 68 wherein the inflammation is associated with Alzheimer's Disease; ALS; an infection; diabetes, asthma; heart disease; arthritis; gout; psoriasis; ulcerative colitis; allergies, chronic obstructive pulmonary disease; myositis; scleroderma; Sjögren's syndrome; lupus; vasculitis; an autoimmune disease; Graves' disease; dermatitis; atherosclerosis; autoinflammatory syndrome; inflammatory bowel disease; atopic dermatitis; Crohn's disease; cancer; chronic stress; alcohol abuse; smoking; or obesity; and combinations thereof.

70. The method of any one of claims 1 to 69, wherein the subject in need thereof is further administered a separate composition comprising a therapeutically-effective amount of colchicine, wherein the colchicine is administered before, contemporaneously with, and/or after administering the transdermal formulation.

71. The method of claim 70, wherein the therapeutically-effective amount of colchicine is administered orally.

72. The method of claim 70, wherein the therapeutically-effective amount of colchicine is administered topically.

73. The method of any one of claims 1 to 69, wherein the transdermal formulation comprising a therapeutically-effective amount of a buffering agent further comprises a therapeutically-effective amount of colchicine.

74. The method of any one of claims 70 to 73, wherein the dosage of the therapeutically-effective amount of colchicine is less than the dosage used to previously treat a gout flare or the therapeutically-effective amount of colchicine is the same as the dosage used to previously treat a gout flare.

75. The method of any one of claims 70 to 74, wherein the therapeutically-effective amount of colchicine comprises from about 0.2 mg to about 4 mg, e.g., about 0.3 mg to about 3.6 mg and/or be present in an amount from 0.02% to about 0.4% w/w of the formulation, e.g., about 0.03% to about 0.36% w/w.

76. The method of any one of claims 1 to 75, wherein transdermal formulation is as described in any of Table 1 to Table 19 or as described elsewhere herein.

77. A transdermal formulation for use in method of treating or reducing inflammation in a subject in need thereof, the transdermal formulation comprising a therapeutically-effective amount of a buffering agent, and comprising a therapeutically-effective amount of a nonsteroidal medicament and/or a corticosteroid,
wherein the transdermal formulation reduces blood or serum levels of C-Reactive Protein relative to a subject that is not administered the combination therapy.

78. A transdermal formulation for use in method of preventing inflammation and/or reducing the severity of upcoming inflammation, the transdermal formulation comprising a therapeutically-effective amount of a buffering agent, and comprising a therapeutically-effective amount of a nonsteroidal medicament and/or a corticosteroid;
wherein the transdermal formulation is administered before symptoms of inflammation are experienced by the subject
wherein preventing inflammation and/or reducing the severity of upcoming inflammation is determined in part by a reduction of blood or serum levels of C-Reactive Protein relative to a subject that is not administered the combination therapy.

79. A transdermal formulation for use in method of reducing the likelihood a recurrent inflammation, the method comprising administering to a subject that previously has been treated for inflammation, the transdermal formulation comprising a therapeutically-effective amount of a buffering agent, and comprising a therapeutically-effective amount of a nonsteroidal medicament and/or a corticosteroid,
wherein the transdermal formulation is administered before symptoms of inflammation are experienced by the subject, and
wherein reducing the likelihood of a recurrent inflammation is determined in part by a reduction of blood or serum levels of C-Reactive Protein relative to a subject that is not administered the combination therapy.

80. A transdermal formulation for use in method of treating chronic inflammation, the method comprising administering to a subject that previously has been treated for inflammation, the transdermal formulation comprising a therapeutically-effective amount of a buffering agent, and comprising a therapeutically-effective amount of a nonsteroidal medicament and/or a corticosteroid,
wherein treating chronic inflammation is determined in part by a reduction of blood or serum levels of C-Reactive Protein relative to a subject that is not administered the combination therapy.

81. A transdermal formulation for use in method of treating a bone density disorder in a subject in need thereof. The method comprising administering to the subject, the transdermal formulation comprising a therapeutically-effective amount of a buffering agent.

82. A transdermal formulation for use in method of treating or reducing inflammation in a subject in need thereof, the transdermal formulation comprising a therapeutically-effective amount of a buffering agent, and comprising a therapeutically-effective amount of colchicine,
wherein the transdermal formulation reduces blood or serum levels of C-Reactive Protein relative to a subject that is not administered the combination therapy.

83. The transdermal formulation of claim 68, wherein the transdermal formulation further reduces or eliminates the need for a rescue medicine; improves the subject's physical function as measured by a Patient-Reported Outcomes Measurement Information System (PROMIS) score, e.g., PROMIS PF 20; provides an improvement in the subject's Sum of Pain Intensity Difference (SPID) score; lowers the subject's pain-numeric rating; decreases the time to resolution of pain relative to a historical control patient; lowers the subject-reported or physician-assessed moderate-to-severe joint tenderness; lowers the subject-reported or physician-assessed moderate-to-severe joint swelling; reduces uric acid crystal levels in blood or plasma; raises urine pH, and/or increases patient satisfaction.

84. A transdermal formulation for use in method of preventing inflammation and/or reducing the severity of upcoming inflammation, the transdermal formulation comprising a therapeutically-effective amount of a buffering agent, and comprising a therapeutically-effective amount of colchicine;
wherein the transdermal formulation is administered before symptoms of inflammation are experienced by the subject
wherein preventing inflammation and/or reducing the severity of upcoming inflammation is determined in part by a reduction of blood or serum levels of C-Reactive Protein relative to a subject that is not administered the combination therapy.

85. A transdermal formulation for use in method of reducing the likelihood a recurrent inflammation, the method comprising administering to a subject that previously has been treated for inflammation, the transdermal formulation comprising a therapeutically-effective amount of a buffering agent, and comprising a therapeutically-effective amount of colchicine,
wherein the transdermal formulation is administered before symptoms of inflammation are experienced by the subject, and
wherein reducing the likelihood of a recurrent inflammation is determined in part by a reduction of blood or serum levels of C-Reactive Protein relative to a subject that is not administered the combination therapy.

86. A transdermal formulation for use in method of treating chronic inflammation, the method comprising administering to a subject that previously has been treated for inflammation, the transdermal formulation comprising a therapeutically-effective amount of a buffering agent, and comprising a therapeutically-effective amount of colchicine,
wherein treating chronic inflammation is determined in part by a reduction of blood or serum levels of C-Reactive Protein relative to a subject that is not administered the combination therapy.

87. A transdermal formulation for use in method of treating a bone density disorder in a subject in need thereof. The method comprising administering to the subject, the transdermal formulation comprising a therapeutically-effective amount of a buffering agent and comprising a therapeutically-effective amount of colchicine.

88. The transdermal formulation of any one of claims 77 to 87, wherein transdermal formulation is as described in any of Table 1 to Table 19 or as described elsewhere herein.
